(19) United States
(12) Patent Application Publication
(10) Pub. No.: US 2007/0161619 A1
(43) Pub. Date: Jul. 12, 2007

(54) Pyrroloquinoline and piperidoquinoline derivatives, preparation thereof, compositions containing them and uses thereof

(75) Inventors: Yun-Jin Hu, Quebec (CA); Miroslaw Tomaszewski, Quebec (CA); Christopher Walpole, Quebec (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 10/597,817

(22) PCT Filed: Feb. 2, 2005
(86) PCT No.: PCT/SE05/00125

§ 371 Date: Aug. 8, 2006

(30) Foreign Application Priority Data
Feb. 10, 2004 (SE) 0400285-3

(51) Int. Cl. A61K 31/541; A61K 31/5377; A61K 31/496; A61K 31/4745; C07D 471/02

(52) U.S. Cl. 514/210.21; 514/291; 514/232.8; 514/227.8; 544/60; 514/253.03; 544/126; 544/361; 546/81

(57) ABSTRACT

Compounds of general formula: 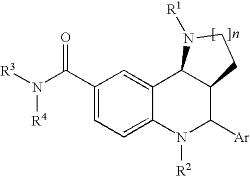
wherein n, R¹, R², R³, R⁴ and Ar are as defined in the specification, as well as salts, enantiomers thereof and pharmaceutical compositions including the compounds are prepared. They are useful in therapy, in particular in the management of pain.

### FIELD OF THE INVENTION

The present invention is directed to novel compounds, processes for their preparation, their uses and pharmaceutical compositions comprising the novel compounds. These compounds are useful in therapy, and in particular for the treatment of pain and disorders related to central nerve systems.

### BACKGROUND OF THE INVENTION

Many GPCR receptors, such as CCK B, BK2, V1a, CB1, CB2, MC3, MC4, MC5, Mt1, GHR-S, H1, 5HT2c, 5HT6, M4, A2a, BRS-3, FPR1, NK1 and Orl1, have been identified to be a contributing factor in regulating many disorders in human being. For example, 5HT2c (human Serotonin subtype 2c) receptor has been linked to anxiety disorders, central nervous system diseases, and major depressive disorders. CB1 and CB2 (Human Cannabinoid) receptors have been linked to pain, glaucoma, epilepsy, obesity and nausea, among other cannabinoid-associated disorders. BK2 (human Bradykinin) receptors have been linked to inflammation, cardiovascular diseases, pain, allergies, asthma and pancreatitis.

It has been found that by regulating these GPCR receptors, one or more above-identified disorders can be properly treated, relieved or cured.

There is a need for compounds that can interact and/or regulate these receptors.

### DESCRIPTION OF THE INVENTION

Accordingly, it is an objective of certain embodiments of the present invention to provide a compound that regulates one or more GPCR receptors.

It is another objective of certain embodiments of the present invention to provide a compound that is useful in treating one or more of the disorders described above.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “alkynyl” used alone or as suffix or prefix, refers to a monovalent straight or branched chain hydrocarbon radical having at least one carbon-carbon triple bond and comprising at least 2 up to about 12 carbon atoms. The triple bond of an alkynyl group can be unconjugated or conjugated to another unsaturated group. Suitable alkynyl groups include, but are not limited to, C₂₋₆alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl can be unsubstituted or substituted with one or two suitable substituents.

The term “cycloalkyl,” used alone or as suffix or prefix, refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkenyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms.

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to links two structures together.

The term “heterocycle” used alone or as a suffix or prefix, refers to a ring-containing structure or molecule having one or more multivalent heteroatoms, independently selected from N, O and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroalkyl” used alone or as a suffix or prefix, refers to a radical formed as a result of replacing one or more carbon atom of an alkyl with one or more heteroatoms selected from N, O and S.

The term “heteroaromatic” used alone or as a suffix or prefix, refers to a ring-containing structure or molecule having one or more multivalent heteroatoms, independently selected from N, O and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

The term “heteroaryl” used alone or as a suffix or prefix, refers to a heterocyclyl having aromatic character.

The term “heterocylcoalkyl” used alone or as a suffix or prefix, refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

The term “heteroarylene” used alone or as a suffix or prefix, refers to a heterocyclylene having aromatic character.

The term “heterocycloalkylene” used alone or as a suffix or prefix, refers to a heterocyclylene that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “substituted” used as a prefix refers to a structure, molecule or group, wherein one or more hydrogens are replaced with one or more C₁₋₁₂hydrocarbon groups, or one or more chemical groups containing one or more heteroatoms selected from N, O, S, F, Cl, Br, I, and P. Exemplary chemical groups containing one or more heteroatoms include heterocyclyl, —NO₂, —OR, —Cl, —Br, —I, —F, —CF₃, —C(═O)R, —C(═O)OH, —NH₂, —SH, —NHR, —NR₂, —SR, —SO₃H, —SO₂R, —S(═O)R, —CN, —OH, —C(═O)OR, —C(═O)NR₂, —NRC(═O)R, oxo (═O), imino (═NR), thio (═S), and oximino (═N—OR), wherein each “R” is a C₁₋₁₂hydrocarbyl. For example, substituted phenyl may refer to nitrophenyl, pyridylphenyl, methoxyphenyl, chlorophenyl, aminophenyl, etc., wherein the nitro, pyridyl, methoxy, chloro, and amino groups may replace any suitable hydrogen on the phenyl ring.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

The term “optionally substituted” refers to both groups, structures, or molecules that are substituted and those that are not substituted.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benzotriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “amine” or “amino” used alone or as a suffix or prefix, refers to radicals of the general formula —NRR′, wherein R and R′ are independently selected from hydrogen or a hydrocarbon radical.

“Acyl” used alone, as a prefix or suffix, means —C(═O)—R, wherein R is an optionally substituted hydrocarbyl, hydrogen, amino or alkoxy. Acyl groups include, for example, acetyl, propionyl, benzoyl, phenyl acetyl, carboethoxy, and dimethylcarbamoyl.

Halogen includes fluorine, chlorine, bromine and iodine.

“Halogenated,” used as a prefix of a group, means one or more hydrogens on the group is replaced with one or more halogens.

“RT” or “rt” means room temperature.

A first ring group being “fused” with a second ring group means the first ring and the second ring share at least two atoms therebetween.

“Link,” “linked,” or “linking,” unless otherwise specified, means covalently linked or bonded.

Provided herein is a compound of formula I, a pharmaceutically acceptable salt thereof, diastereomers thereof, enantiomers thereof, and mixtures thereof: 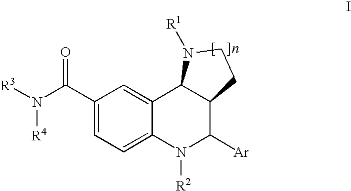
wherein

n is 1 or 2;

R¹ is selected from —H, C₁₋₆alkyl, C₂₋₆alkenyl, C₃₋₆cycloalkyl, —CH₂—R⁸, —C(═O)—NH—R⁷, —C(═S)—NH—R⁷, —C(═O)—O—R⁷, —S(═O)₂—R⁶, and —C(═O)—R⁵, wherein R⁵, R, R⁷ and R⁸ are independently selected from C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl used in defining R¹, R⁵, R⁶, R⁷ or R⁸ are optionally substituted with one or more groups selected from —OH, —CHO, —NH₂, —NHR, —NR₂, C₁₋₆alkyl, —C(═O)—R, —C(═O)—OR, —C(═O)—NHR, —SR, —SH, halogenated C₁₋₆alkyl, —CN, —NO₂, C₁₋₆alkoxy and halogen, or disubstituted with —O—CH₂—O— to form a fused ring;

R² is selected from —H and C₁₋₆alkyl;

R³ and R⁴ are independently selected from —H, C₁₋₆alkyl, C2-6alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy and halogen; or R³ and R⁴ together with the nitrogen connected thereto in formula I form a heterocycle ring, wherein said heterocycle ring is optionally substituted with one or more groups selected from benzyl, —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen;

Ar is selected from C₆₋₁₀aryl and C₃₋₆heteroaryl, wherein said C₆₋₁₀aryl and C₃₋₆heteroaryl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen; and

R is C₁₋₆alkyl.

In one embodiment, the compounds of the present invention are those of formula I,

wherein n is 1 or 2;

R¹ is selected from C₁₋₆alkyl, C₂₋₆alkenyl, C₃₋₆cycloalkyl, —CH₂—R⁸, —C(═O)—NH—R⁷, —C(═S)—NH—R⁷, —S(═O)₂—R⁶, and —C(═O)—R⁵, wherein R⁵, R⁶, R⁷ and Rare independently selected from C₁₋₆alkyl, C₂₋₆alkenyl, C₃₋₆cycloalkyl, C₃₋₆cycloalkyl-C₁₋₂alkyl, phenyl, phenyl-C₁₋₂alkyl, C₃₋₆heterocycloalkyl, C₃₋₆heterocycloalkyl-C₁₋₂alkyl, C₃₋₆heteroaryl, and C₃₋₆heteroaryl-C₁₋₂alkyl, wherein said C₁₋₄alkyl, C₂₋₄alkenyl, C₃₋₆alkyl, phenyl, phenyl-C₁₋₂alkyl, C₃₋₆heterocycloalkyl, C₃₋₆heterocycloalkyl-C₁₋₂alkyl, C₃₋₆heteroaryl, and C₃₋₆heteroaryl-C₁₋₂alkyl used in defining R¹, R⁵, R⁶, R⁷ or R⁸ are optionally substituted with one or more groups selected from —OH, —CHO, —NH₂, —NHR, —NR₂, C₁₋₃alkyl, —C(═O)—R, —C(═O)—OR, —SR, —CF₃, —CN, methoxy, ethoxy, fluoro and chloro, or disubstituted with —O—CH₂—O— to form a fused ring;

R² is selected from —H, methyl and ethyl;

R³ and R⁴ are independently selected from —H, C₁₋₄alkyl, C₂₋₄alkenyl, C₃₋₆cycloalkyl, C₃₋₆cycloalkyl-C₁₋₂alkyl, phenyl, phenyl-C₁₋₂alkyl, C₃₋₆heterocycloalkyl, C₃₋₆heterocycloalkyl-C₁₋₂alkyl, C₃₋₆heteroaryl, and C₃₋₆heteroaryl-C₁₋₂alkyl, wherein said C₁₋₄alkyl, C₂₋₄alkenyl, C₃₋₆cycloalkyl, C₃₋₆cycloalkyl-C₁₋₂alkyl, phenyl, phenyl-C₁₋₂alkyl, C₃₋₆heterocycloalkyl, C₃₋₆heterocycloalkyl-C₁₋₂alkyl, C₃₋₆heteroaryl, and C₃₋₆heteroaryl-C₁₋₂alkyl are optionally substituted with one or more groups selected from —CHO, —NH₂, —NHR, —NR₂, C₁₋₃alkyl, —C(═O)—OR, —CF₃, —CN, methoxy, ethoxy, fluoro and chloro; or R³ and R⁴ together with the nitrogen connected thereto in formula I form a heterocycloalkyl ring, wherein said heterocycloalkyl ring is optionally substituted with one or more groups selected from benzyl, —CHO, C₁₋₃alkyl, —C(═O)—OR, —CF₃, —CN, methoxy, ethoxy, fluoro and chloro;

Ar is selected from phenyl and five or six-membered C₃₋₅heteroaryl, wherein said phenyl and five or six-membered C₃₋₅heteroaryl are optionally substituted with one or more groups selected from C₁₋₃alkyl, —C(═O)—OR, —CF₃, —CN, methoxy, ethoxy, fluoro and chloro; and

R is C₁₋₃alkyl.

In another embodiment, the compounds of the present invention are those of formula I,

wherein n is 1 or 2;

R¹ is selected from —CH₂—R⁸, —C(═O)—NH—R⁷, —C(═S)—NH—R⁷, —S(═O)₂—R⁶, and —C(═O)—R⁵, wherein R⁵, R⁶, R⁷ and R⁸ are independently selected from C₁₋₆alkyl, C₂₋₆alkenyl, C₃₋₆cycloalkyl, C₃₋₆cycloalkyl-C₁₋₂alkyl, phenyl, benzyl, C₃₋₆heterocycloalkyl, C₃₋₆heterocycloalkyl-C₁₋₂alkyl, C₃₋₆heteroaryl, wherein said C₁₋₆alkyl, C₂₋₆alkenyl, C₃₋₆cycloalkyl, C₃₋₆cycloalkyl-C₁₋₂alkyl, phenyl, benzyl, C₃₋₆heterocycloalkyl, C₃₋₆heterocycloalkyl-C₁₋₂alkyl, C₃₋₆heteroaryl are optionally substituted with one or more groups selected from methyl, ethyl, —C(═O)—CH₃, —C(═O)—OCH₃, —C(═O)—OCH₂—CH₃, —SCH₃, —CN, methoxy, ethoxy, fluoro and chloro, or said phenyl or benzyl is optionally disubstituted with —O—CH₂—O— to form a fused ring;

R² is selected from —H, methyl and ethyl;

R³ and R⁴ are independently selected from —H, methyl, ethyl, propenyl, cyclopropyl-methyl, cyclobutyl, cyclopentyl, tetrahydrofuryl-methyl, furyl-methyl, pyridyl-methyl, thiomorpholinyl-ethyl, pyrrolidinyl-methyl, pyrrolidinyl-ethyl, thienyl-methyl, wherein said methyl, ethyl, propenyl, cyclopropyl-methyl, cyclobutyl, cyclopentyl, tetrahydrofuryl-methyl, furyl-methyl, pyridyl-methyl, thiomorpholinyl-ethyl, pyrrolidinyl-methyl, pyrrolidinyl-ethyl, thienyl-methyl are optionally substituted with one or more groups selected from dimethylamino, diethylamino, diisopropylamino, methyl, ethyl, methoxy, or R³ and R⁴ together with the nitrogen connected thereto in formula I form a heterocycloalkyl ring selected from piperidine, azetidine, piperazine, pyrrolidine and morpholine, wherein said piperidine, azetidine, piperazine, pyrrolidine and morpholine is optionally substituted with one or more groups selected from benzyl, methyl and —CHO; and

Ar is selected from phenyl, pyridyl, furyl and thienyl, wherein said phenyl, pyridyl, furyl and thienyl are optionally substituted with one or more methoxy or ethoxy.

In a further embodiment, the compounds of the present invention are those of formula I, wherein n is 1 or 2;

R¹ is selected from —CH₂—R⁵, —C(═O)—NH—R⁷, —C(═S)—NH—R⁷, —S(═O)₂—R⁶, and —C(═O)—R⁵, wherein R⁵, R⁶, R⁷ and R⁸ are independently selected from methyl, ethyl, isopropyl, 1-propyl, 2-methyl-1-propyl, 3-methyl-1-butyl, 2-ethyl-1-butyl, 1-butyl, 1-propen-3-yl, 4-methyl-2-penten-1-yl, 3-methyl-2-buten-1-yl, cyclopropyl, cyclobutyl, cyclopentyl, cyclohexyl, cyclopentyl-methyl, phenyl, benzyl, 4-morpholinyl-ethyl, tetrahydrothiopyran-4-yl-ethyl, furyl, isoxazolyl, pyridyl, thienyl, pyrazolyl, imidazolyl, and pyrrolyl, wherein said methyl, ethyl, isopropyl, 1-propyl, 2-methyl-1-propyl, 3-methyl-1-butyl, 2-ethyl-1-butyl, 1-butyl, 1-propen-3-yl, 4-methyl-2-penten-1-yl, 3-methyl-2-buten-1-yl, cyclopropyl, cyclobutyl, cyclopentyl, cyclohexyl, cyclopentyl-methyl, phenyl, benzyl, 4-morpholinyl-ethyl, tetrahydrothiopyran-4-yl-ethyl, furyl, isoxazolyl, pyridyl, thienyl, pyrazolyl, imidazolyl, and pyrrolyl are optionally substituted with one or more groups selected from methyl, ethyl, —C(═O)—CH₃, —C(═O)—OCH₃, —C(═O)—OCH₂—CH₃, —SCH₃, —CN, methoxy, ethoxy, fluoro and chloro, or said phenyl or benzyl is optionally disubstituted with —O—CH₂—O— to form a fused ring;

R² is selected from —H, methyl and ethyl;

R³ and R⁴ are independently selected from —H, methyl, ethyl, propenyl, cyclopropyl-methyl, cyclobutyl, cyclopentyl, tetrahydrofuryl-methyl, furyl-methyl, pyridyl-methyl, thiomorpholinyl-ethyl, pyrrolidinyl-methyl, pyrrolidinyl-ethyl, thienyl-methyl, wherein said methyl, ethyl, propenyl, cyclopropyl-methyl, cyclobutyl, cyclopentyl, tetrahydrofuryl-methyl, furyl-methyl, pyridyl-methyl, thiomorpholinyl-ethyl, pyrrolidinyl-methyl, pyrrolidinyl-ethyl, thienyl-methyl are optionally substituted with one or more groups selected from dimethylamino, diethylamino, diisopropylamino, methyl, ethyl, methoxy, or R³ and R⁴ together with the nitrogen connected thereto in formula I form a heterocycloalkyl ring selected from piperidine, azetidine, piperazine, pyrrolidine and morpholine, wherein said piperidine, azetidine, piperazine, pyrrolidine and morpholine is optionally substituted with one or more groups selected from benzyl, methyl and —CHO; and

Ar is selected from phenyl, 4-ethoxyphenyl, 4-methoxyphenyl, pyridyl, furyl and thienyl.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of Formula I. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I. It will further be understood that the present invention encompasses tautomers of the compounds of the formula I.

It will also be understood that certain compounds of the present invention may exist in solvated, for example hydrated, as well as unsolvated forms. It will further be understood that the present invention encompasses all such solvated forms of the compounds of the formula I.

Within the scope of the invention are also salts of the compounds of the formula I. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of formula I above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

The novel compounds of the present invention are useful in therapy, especially for the treatment of various pain conditions such as chronic pain, neuropathic pain, acute pain, cancer pain, pain caused by rheumatoid arthritis, migraine, visceral pain etc. This list should however not be interpreted as exhaustive.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of opioid receptors is present or implicated in that paradigm. This may involve the use of isotopically labelled versions of the compounds of the invention in diagnostic techniques and imaging applications such as positron emission tomography (PET).

Compounds of the invention are useful for the treatment of glaucoma, epilepsy and nausea, inflammation, cardiovascular diseases, allergies, asthma and pancreatitis, diarrhoea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastrointestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following myocardial infarction, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

Also within the scope of the invention is the use of any of the compounds according to the formula I above, for the manufacture of a medicament for the treatment of any of the conditions discussed above.

A further aspect of the invention is a method for the treatment of a subject suffering from any of the conditions discussed above, whereby an effective amount of a compound according to the formula I above, is administered to a patient in need of such treatment.

Thus, the invention provides a compound of formula I, or pharmaceutically acceptable salt or solvate thereof, as hereinbefore defined for use in therapy.

In a further aspect, the present invention provides the use of a compound of formula I, or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be construed accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, intracerebroventricularly and by injection into the joints.

In one embodiment of the invention, the route of administration may be orally, intravenously or intramuscularly.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid and liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of formula I as defined above for the manufacture of a medicament.

Also within the scope of the invention is the use of any compound of formula I for the manufacture of a medicament for the therapy of pain.

Additionally provided is the use of any compound according to Formula I for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the formula I above, is administered to a patient in need of such therapy.

Additionally, there is provided a pharmaceutical composition comprising a compound of Formula I, or a pharmaceutically acceptable salt thereof, in association with a pharmaceutically acceptable carrier.

Particularly, there is provided a pharmaceutical composition comprising a compound of Formula I, or a pharmaceutically acceptable salt thereof, in association with a pharmaceutically acceptable carrier for therapy, more particularly for therapy of pain.

Further, there is provided a pharmaceutical composition comprising a compound of Formula I, or a pharmaceutically acceptable salt thereof, in association with a pharmaceutically acceptable carrier use in any of the conditions discussed above.

Also provided herein is a method of preparing a compound of formula I.

In one embodiment, the invention provides a process for preparing a compound of formula I, comprising:

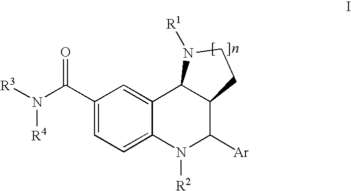

reacting a compound of formula II with a compound selected from R⁵—C(═O)—Cl, R⁶—S(═O)₂—Cl, R⁷—NCO, R⁷—NCS and R⁸CHO: 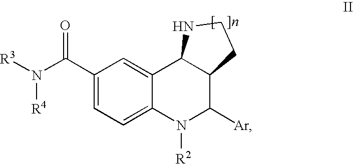
wherein

n is 1 or 2;

R¹ is selected from —CH₂—R¹, —C(═O)—NH—R⁷, —C(═S)—NH—R⁷, —S(═O)₂—R⁶, and —C(═O)—R⁵, wherein R⁵, R⁶, R⁷ and R⁸ are independently selected from C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl are optionally substituted with one or more groups selected from —OH, —CHO, —NH₂, —NHR, —NR₂, C₁₋₆alkyl, —C(═O)—R, —C(═O)—OR, —C(═O)—NHR, —SR, —SH, halogenated C₁₋₆alkyl, —CN, —NO₂, C₁₋₆alkoxy and halogen, or disubstituted with —O—CH₂—O— to form a fused ring;

R² is selected from —H and C₁₋₆alkyl;

R³ and R⁴ are independently selected from —H, C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy and halogen; or R³ and R⁴ together with the nitrogen connected thereto in formula I form a heterocycle ring, wherein said heterocycle ring is optionally substituted with one or more groups selected from benzyl, —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen;

Ar is selected from C₆₋₁₀aryl and C₃₋₆heteroaryl, wherein said C₆₋₁₀aryl and C₃₋₆heteroaryl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen; and

R is C₁₋₆alkyl.

In another embodiment, the invention provides a process for preparing a compound of formula I, comprising:

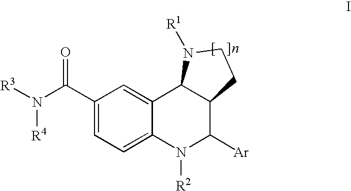

reacting a compound of formula III with R³R⁴NH: 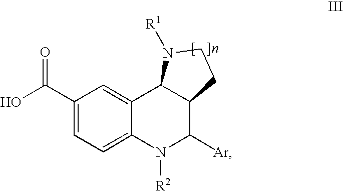
wherein

n is 1 or 2;

R¹ is selected from —C(═O)—O—C₁₋₆alkyl and —C(═O)—O—C₂₋₆alkenyl;

R² is selected from —H and C₁₋₆alkyl;

R³ and R⁴ are independently selected from —H, C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy and halogen; or R³ and R⁴ together with the nitrogen connected thereto in formula I form a heterocycle ring, wherein said heterocycle ring is optionally substituted with one or more groups selected from benzyl, —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen;

Ar is selected from C₆₋₁₀aryl and C₃₋₆heteroaryl, wherein said C₆₋₁₀aryl and C₃₋₆heteroaryl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen; and

R is C₁₋₆alkyl.

In a further embodiment, the invention provides a process for preparing a compound of formula IV, comprising:

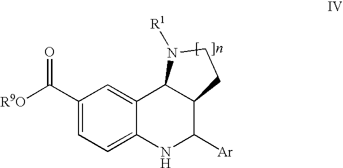

reacting a compound of formula V with a compound of formula VI: 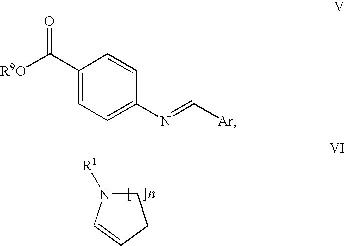
wherein

n is 1 or 2;

R¹ is selected from —C(═O)—O—C₁₋₆alkyl and —C(═O)—O—C₂₋₆alkenyl;

R⁹ is C₁₋₆alkyl;

Ar is selected from C₆₋₁₀aryl and C₃₋₆heteroaryl, wherein said C₆₋₁₀aryl and C₃₋₆heteroaryl are optionally substituted with one or more groups selected from —OH, —CHO, —NH₂, —NHR, —NR₂, C₁₋₆alkyl, —C(═O)—OR, —C(—O)—NHR, —SR, —SH, halogenated C₁₋₆alkyl, —CN, —NO₂, C₁₋₆alkoxy, and halogen; and

R is C₁₋₆alkyl.

Particularly, the compounds of the present invention and intermediates used for the preparation thereof can be prepared according to the synthetic routes as exemplified in Schemes 1-3 and General Procedures 1-11, wherein unless otherwise defined, Ar, R²⁻⁸ and n are defined as above. 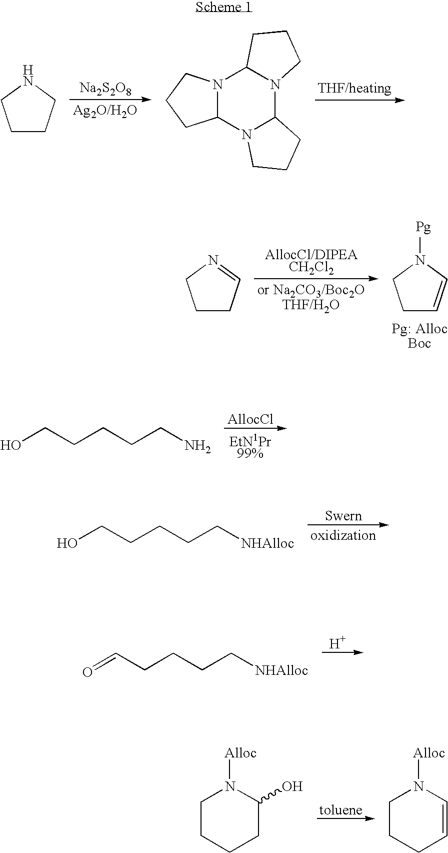 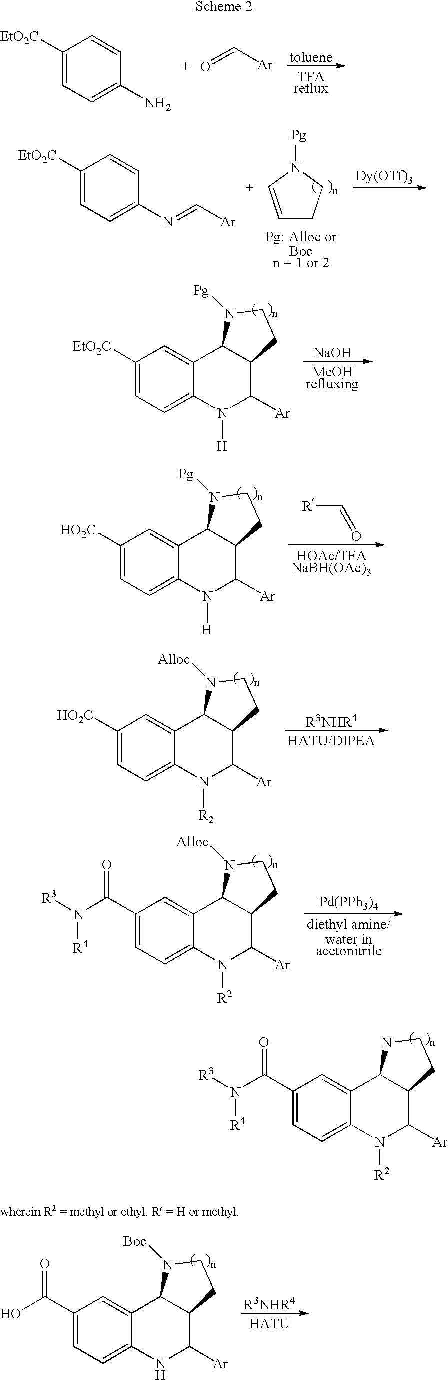 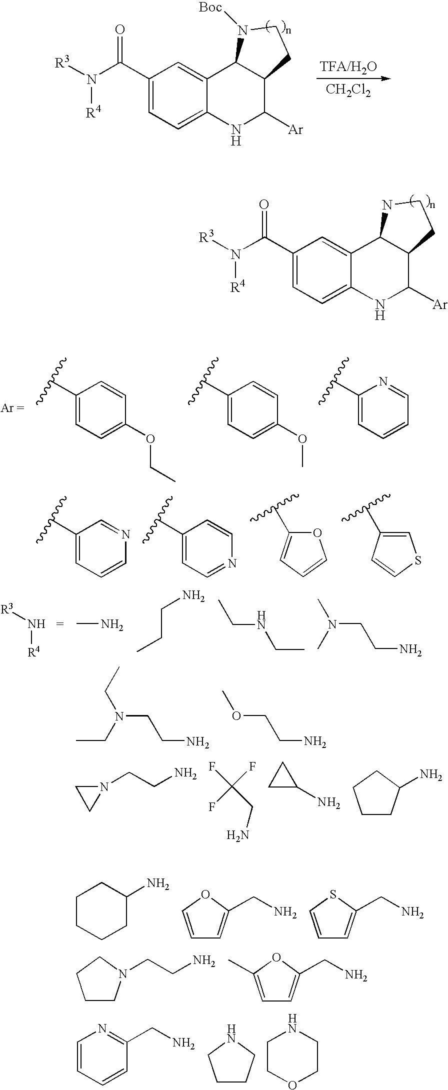 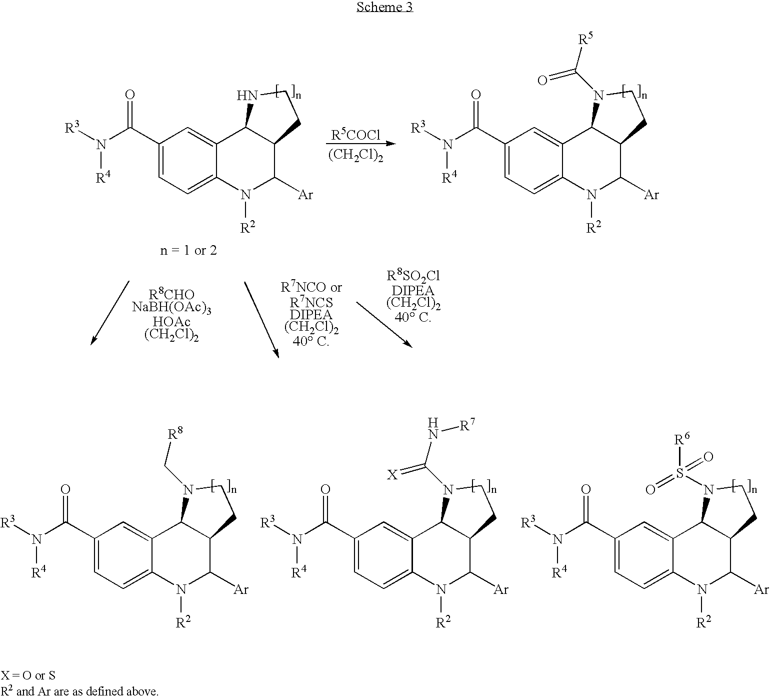
General Procedure 1 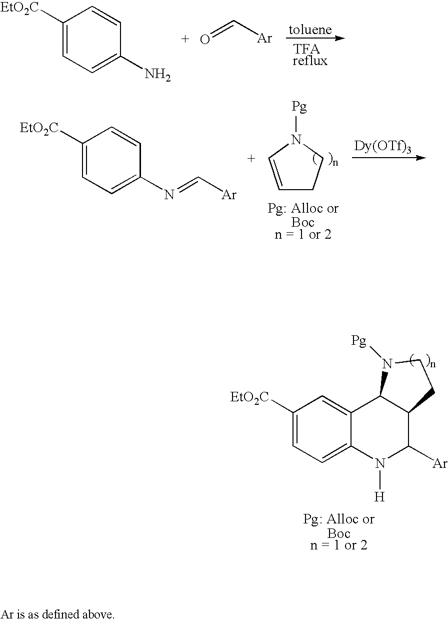
Ethyl 4-aminobenzoate (1 equiv.), aldehyde (1.1 equiv.), in dry toluene was added a drop of TFA. The solution was refluxed for overnight, while water was removed by Dean Stark trap. After removal of the solvent, the resulted Schiff base was used for next step directly. To the residue was added alkyl 2,3-dihydro-1H-pyrrole-1-carboxylate or other reactant as shown in above scheme (1.1 equivalent) in acetonitrile. The reaction mixture was stirred at room temperature for 16 hours. The solvent was removed to give a residue, which was purified by silica gel column chromatography giving the desired compound at approximately 1:1 ratio.
General Procedure 2 (Saponification of the ethyl ester) 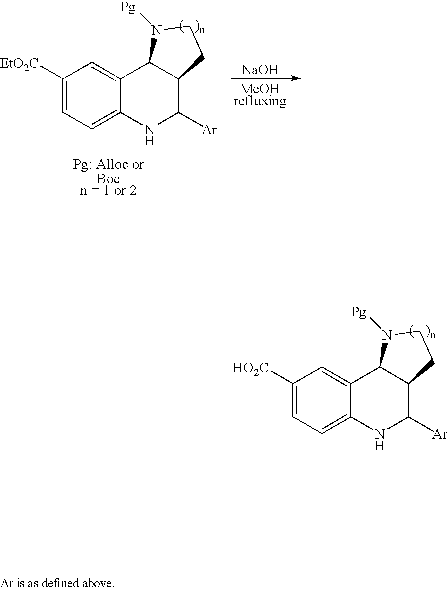
To the starting material, ethyl acetate (1 equiv.) in methanol was added 0.5N aqueous NaOH(H₂O/MeOH: 1:2). The solution was refluxed overnight in the nitrogen atmosphere. The reaction solution was neutralized with 10% HCl. Then, the solvent was removed. The slurry was extracted with ethyl acetate and washed with water and brine. The dried organic phase was concentrated to give a residue, which was purified by Flash chromatography. The product contains two diastereomers in approximately 1:1 ratio.
General Procedure 3 (Saponification of the ethyl ester) 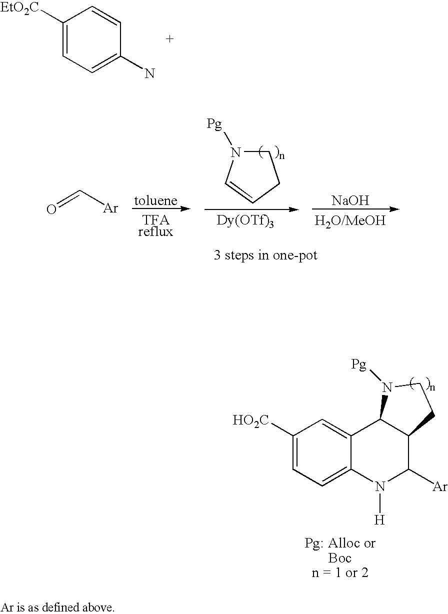
The Schiff base formation and cyclization step were the same as described in the general procedure 1. The solvent was removed and the residue was used directly in next step. The residue was treated with methanol and 0.5 N aqueous NaOH(H₂O/MeOH: 1:2) at reflux for overnight. The reaction mixture was neutralized with 10% HCl, and then concentrated in vacuo. The resultant slurry was extracted with ethyl acetate and washed with water and brine. The organic phase was dried and concentrated in vacuo. The product mixture was purified by flash chromatography to afford a mixture of diastereomers in approximately 1:1 ratio.
General Procedure 4 (Alkylation of the aniline) 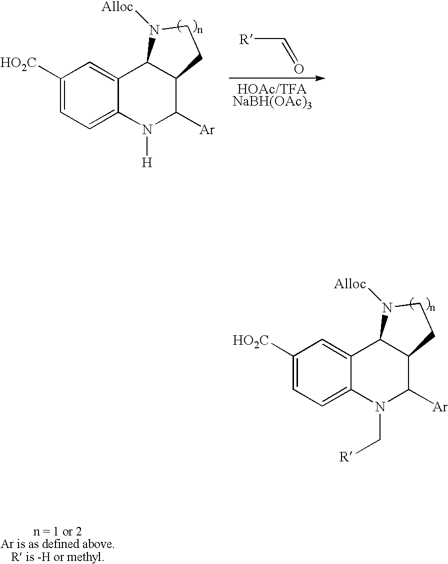
The solution of the starting material aniline (1 equiv.), aldehyde (100 equiv.), HOAc (100 equiv.), TFA (10 equiv.) in CH₂Cl₂, were added NaBH(OAc)₃ (10 equiv.) portion by portion over 45 minutes. Then the solvents were removed to give a residue, which was purified by flash chromatography.
General Procedure 5 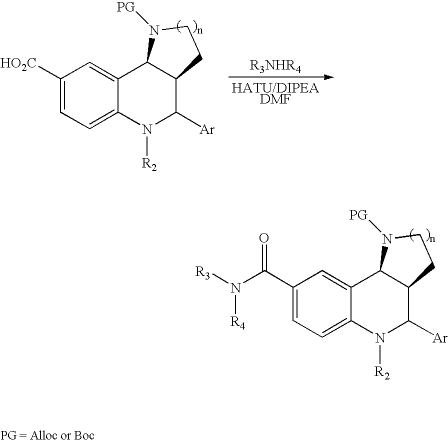
A mixture of carboxylic acid (1 equiv.), HATU (1.1 equiv.), DIPEA (1.1 equiv.) in DMF was stirred for 5 minutes. Then, a primary or secondary amine was added to the solution. The reaction mixture was stirred at room temperature for 4 hours. The solvent was removed in vacuo. The residue was purified by flash column chromatography.
General Procedure 6 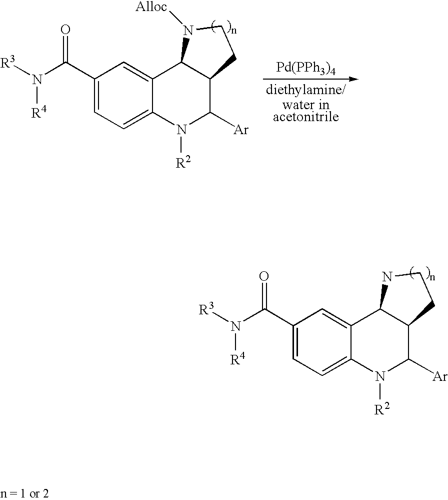
An alloc carbomate (˜1 equiv.), tetrakis(triphenylphosphine)palladium(0) (0.025 equiv.) in water and acetonitrile (1:10) was added diethyl amine (20 equiv.). The reaction was stirred for 4 hours at room temperature. Afterwards, another portion (4.34 mg, 0.025 equiv.) of palladium catalyst was added into the reaction solution. After removal of solvents, the residue was dissolved in CH₂Cl₂ and the solution was treated with of p-TsOH resin (5 equiv.). After 2 hours of stirring the mixture, the resin was filtered and washed with CH₂Cl₂ (3 times) and methanol (3 times). The product was then released from resin by treatment with 1N ammonia in methanol twice. The collected filtrate was dried in in vacuo to give the product as a mixture of two diastereomers in approximately 1:1 ratio.
General Procedure 7 (Boc deprotection) 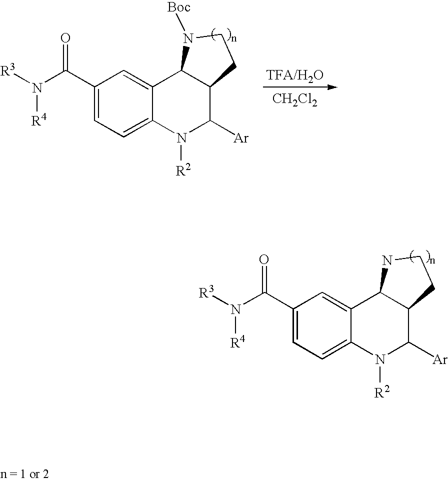
The substrate (1 equiv.) was dissolved in dichloromethane, to which was added TFA/H₂O (1:1, 10% in CH₂Cl₂). The solution was stirred at 40⁰C for 30 minutes. Then the solvents were removed in vacuo. The residue was treated with TFA/H₂O (1:1, 10% in CH₂Cl₂), the solvent removed in vacuo and treated again with TFA/H₂O (1:1, 10% in CH₂Cl₂) and concentrated in vacuo. The residue was dried over vacuum pump to afford the product as TFA salt of a mixture of two diastereomers in approximately 1:1 ratio.
General Procedure 8 (reductive amination) 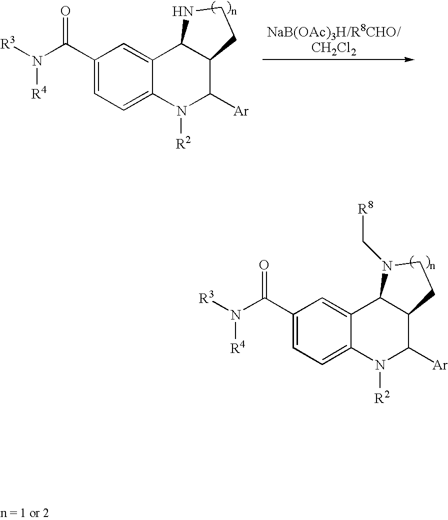
Amine (1 equiv.), aldehyde (2 equiv.), and NaBH(OAc)₃ (2 equiv.) in acetic acid (5 equiv.) and CH₂Cl₂ was stirred at room temperature overnight. After removal of the solvent, the residue was purified by flash chromatography giving a mixture of two diastereomers in approximately 1:1 ratio.
General Procedure 9 (amide formation) 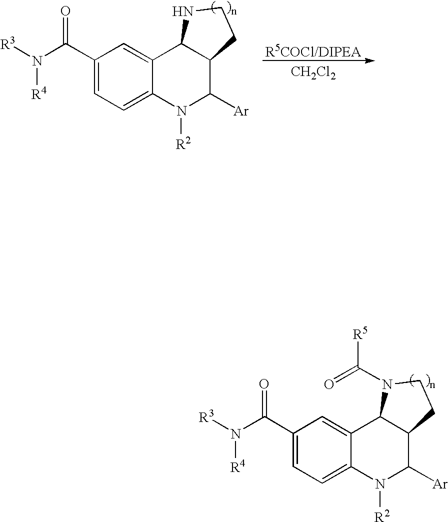
To a dichloromethane solution of amine (1 equiv.) was added acyl chloride (1.2 equiv.) and DIPEA (2 equiv.) in CH₂Cl₂. The reaction was stirred at room temperature for 2 hours. Then the reaction solution was extracted with CH₂Cl₂ after quenched with water. The organic phase was washed with water, 5% NaOH, and brine. The dried organic phase was concentrated to give a residue, which was purified by flash chromatography. A mixture of two diastereomers in approximately 1:1 ratio was obtained.
General Procedure 10 (sulphonyl amide formation) 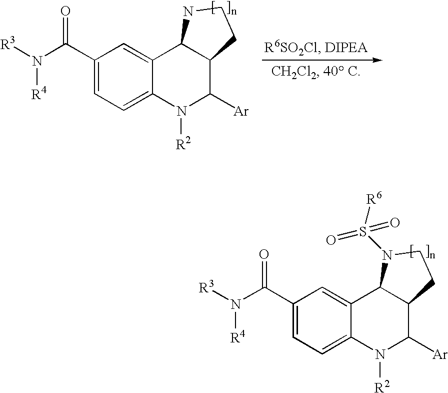
To amine (1 equiv.) in DIPEA (2 equiv.) and CH₂Cl₂, was added sulfonyl chloride (1.2 equiv.) in CH₂Cl₂. The solution was stirred at room temperature for 4 hours. Then the reaction solution was extracted with CH₂Cl₂ after quenched with water. The organic phase was washed with water, 5% NaOH, and brine. The dried organic phase was concentrated to give a white solid, which was purified by flash chromatography. Products were a mixture of two diastereomers in approximately 1:1 ratio.
General Procedure 11 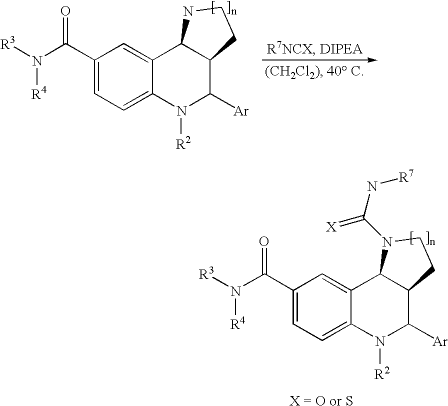
To the amine (1 equiv.) and DIPEA (3 equiv.) in (CH₂Cl)₂ was added isocyanate or thioisocyanate (3 equiv.). The reaction solution was stirred at 40⁰C for 8 hours. Then the reaction solution was extracted with CH₂Cl₂. The organic phase was washed with water, 5% NaOH, and brine. The dried organic phase was concentrated to give a residue, which was purified by flash chromatography. Products were a mixture of two diastereomers in approximately 1:1 ratio.

Accordingly, in another aspect, the present invention provides a compound of formula II: 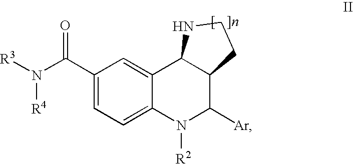
wherein

n is 1 or 2;

R¹ is selected from —H and C₁₋₆alkyl;

R³ and R⁴ are independently selected from —H, C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy and halogen; or R³ and R⁴ together with the nitrogen connected thereto in formula I form a heterocycle ring, wherein said heterocycle ring is optionally substituted with one or more groups selected from benzyl, —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen;

Ar is selected from C₆₋₁₀aryl and C₃₋₆heteroaryl, wherein said C₆₋₁₀aryl and C₃₋₆heteroaryl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen; and

R is C₁₋₆alkyl.

Biological Evaluation

B2 bradykinin

A. hB2 receptor expression and membrane preparation

The cloned human Bradykinin B2 (hB2) receptor in the pCIN vector was purchased from Receptor Biology. The hB2 receptor was stably transfected into HEK 293 S cells and a clonal cell line was generated. Cells were grown in T-flasks with DMEM culture media containing 10% FBS, 2 mM glutamine, 600 μg/ml neomycin and an antibiotic cocktail (100 IU penicillin, 100 μg/ml streptomycin, 0.25 μg/ml amphotericin B). Membranes, expressing the hB2 receptor, were prepared from this cell line according this protocol: Cells are harvested at 1 to 1.2 million cells/ml, pelleted, and resuspended in ice-cold lysis buffer (50 mM Tris, pH 7.0, 2.5 mM EDTA, with PMSF added just prior to use to 0.5 mM from a 0.5 M stock in DMSO. After lysis on ice for 15 min, the cells are homogenized with a polytron for 10 sec. The suspension is spun at 1000 g for 10 min at 4° C. The supernatant is saved on ice and the pellets resuspended and spun as before. The supernatants from both spins are combined and spun at 46,000 g for 10-30 min. The pellets are resuspended in cold Tris buffer (50 mM Tris/Cl, pH 7.0) at a dilution of 0.2-1 ml per 40 million cells and spun again. The final pellets are resuspended in membrane buffer (50 mM Tris, 0.32 M sucrose, pH 7.0). Aliquots are frozen in dry ice/ethanol and stored at −70° C. until use. The protein concentrations are determined by a modified Lowry with SDS.

B. hB2 receptor binding

Membranes expressing the hB2 receptor are thawed at 37° C., passed 3 times through a 25-gauge blunt-end needle, diluted in the bradykinin binding buffer (50 mM Tris, 3 mM MgCl₂, and 1 mg/ml BSA, pH 7.4, 0.02 mg/ml Phenanthroline, 0.25 mg/ml Pefabloc) and 80 μL aliquots containing the appropriate amount of protein (final concentration of 0.25 μg/ml) are distributed in 96-well polystyrene plates (Treff Lab). The IC50 of compounds are evaluated from 10-point dose-response curves, where the serial dilutions are done on a final volume of 150 μL, with 70 μL of ¹²⁵I-Desamino-TyrHOE140 (Kd=0.05) at 50,000 to 60,000 dpm per well (0.03-0.04 nM) in a final volume of 300 μl. The total and non-specific binding are determined in the absence and presence of 0.1 μM (150 μL) of Bradykinin respectively. The plates are vortexed and incubated for 60 minutes at room temperature, filtered through Unifilters-96 GF/B (Canberra Packard), which were presoaked in 0.1% polyethyleneimine, with a harvester using 3 ml of wash buffer (50 mM Tris, pH 7.0, 3 mM MgCl₂). The filters are dried for 1 hour at 55° C. The radioactivity (cpm) is counted in a TopCount (Canberra Packard) after adding 65 μl/well of MS-20 scintillation liquid (Canberra Packard). Compounds of the present invention have demonstrated hB2 receptor binding at concentrations less than 10 μM.

hCB1 and hCB2 receptor binding

Human CB1 (from Receptor Biology) or CB2 (from BioSignal) membranes are thawed at 37° C., passed 3 times through a 25-gauge blunt-end needle, diluted in the cannabinoid binding buffer (50 mM Tris, 2.5 mM EDTA, 5 mM MgCl₂, and 0.5 mg/mL BSA fatty acid free, pH 7.4) and aliquots containing the appropriate amount of protein are distributed in 96-well plates. The IC50 of compounds at hCB1 and hCB2 are evaluated from 10-point dose-response curves done with ³H-CP55,940 at 20000 to 25000 dpm per well (0.17-0.21 nM) in a final volume of 300 μl. The total and non-specific binding are determined in the absence and presence of 0.2 μM of HU210 respectively. The plates are vortexed and incubated for 60 minutes at room temperature, filtered through Unifilters GF/B (presoaked in 0.1% polyethyleneimine) with the Tomtec or Packard harvester using 3 mL of wash buffer (50 mM Tris, 5 mM MgCl₂, 0.5 mg BSA pH 7.0). The filters are dried for 1 hour at 55° C. The radioactivity (cpm) is counted in a TopCount (Packard) after adding 65 μl/well of MS-20 scintillation liquid.

Many of the compounds described in the present invention are found to have an IC50 (dissociating constant) toward B2 receptors of less than 1000 nM.

### EXAMPLES

The invention will further be described in more detail by the following Examples which describe methods whereby compounds of the present invention may be prepared, purified, analyzed and biologically tested, and which are not to be construed as limiting the invention.

### Allyl 2,3-dihydro-1H-pyrrole-1-carboxylate

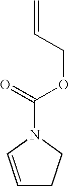 Above compound was prepared by the following method. 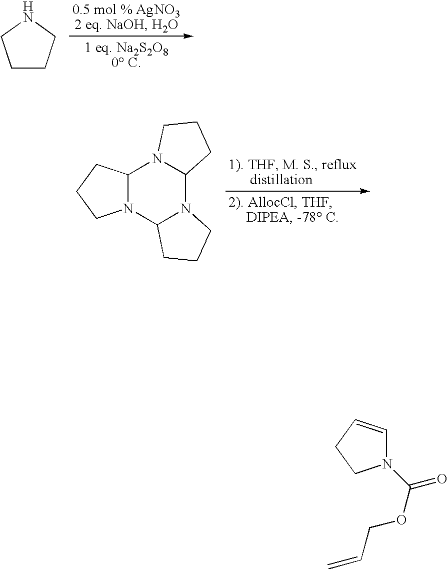
A 25% aqueous solution of sodium persulfate (150 mmol) was added dropwise to a stirred solution of pyrrolidine (150 mmol), sodium hydroxide (12.0 g, 300 mmol) and silver nitrate (0.75 mmol) in water (150 mL) at 0⁰C over 1 hour. After the addition was completed, the reaction mixture was stirred at 4 to 10⁰C for 2.5 hours. Brine was added and the reaction mixture was extracted with CH₂Cl₂ (4×100 mL). The organic phase was dried over sodium sulfate and the solvent was removed under vacuum. The residue was dissolved in THF (500 mL), which was dried with 20 grams of 4A⁰ molecular sieves. Then the solution was distilled in oil bath (110⁰C) through a short path distillation apparatus into a flask cooled to −78⁰C. Diisoporpanylethyl amine (150 mmol) was added then alkyl chloroformate (100 mmol) dropwise. The suspension was allowed to warm up to room temperature overnight. The reaction solution was washed with water and brine. The dried solution was concentrated to give a residue, which was further purified by Flash chromatography. Product: 7.5 g, yield: 33%.

¹H NMR (400 MHz, CDCl₃): 6.53 (1H, m), 5.92 (1H, m), 5.230 (1H, dd, J=17.4, 1.5 Hz), 5.18 (1H, dd, J=10.5, 1.5 Hz), 5.90 (1H,m), 5.03 (1H, m), 4.58 (2H, m), 3.73 (2H, m), 2.63 (2H, m). MS (ESI) (M+H)⁺=153.18.

### Allyl 3,4-dihydropyridine-1(2H)-carboxylate

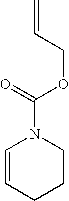 Above compound was prepared by following literature method. (See Osamu Okitsu, Ritsu Suzuki, and Shuj Kobayashi, J. Org. Chem. 2001, 66, 809-823) MS (ESI) (M+H)⁺=168.2.

### Example 1

### Allyl-9-[(diethylamino)carbonyl]-5-(4-ethoxyphenyl)-3,4,4a,5,6,10b-hexahydrobenzo[h]-1,6-naphthyridine-1(2H)-carboxylate

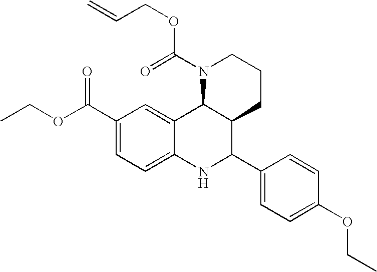 The titled compound was obtained by following the general procedure 1 (7.0 g, yield: 81%). MS (ESI) (M+H)⁺=465.563.

### 1-[(Allyloxy)carbonyl]-5-(4-ethoxyphenyl)-1,2,3,4,4a,5,6,10b-octahydrobenzo[h]-1,6-naphthyridine-9-carboxylic acid

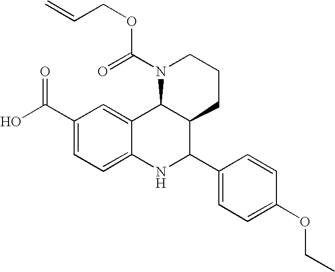 The titled product (6.5 g; yield, 99%) was obtained by following the general procedure 2. ¹H NMR (400 MHz, CDCl₃): 8.28 (0.45H, d, J=1.4 Hz), 8.23 (0.55H, d, J=1.4 Hz), 7.82 (0.55H, dd, J=8.6, 1.4 Hz), 7.79 (0.45H, d, J=8.6 Hz), 7.32 (1H, d, J=8.6 Hz), 7.12 (1H,d, J=8.6 Hz), 6.83 (1H,d, J=8.6 Hz), 6.57 (1H,d, J=8.6 Hz), 6.01 (1H, m), 5.35 (1H,m), 5.23 (0.55H, m), 4.89 (1H, m), 4.76 (1H, m), 4.65 (1H, m), 4.42 (1H,d, J=2.38 Hz), 4.05 (0.9H, q, J=−7.0 Hz, 3.99 (1.1H, q, J=7.0 Hz), 3.55 (0.45H, m), 3.40 (1.55H, m), 2.55 (1H, m), 2.13 (0.55H, m), 2.01 (1.0H, m), 1.62 (0.45H, m), 1.43 (1.25H, t, J=7.0 Hz), 1.39 (1.65H, t, J=7.0 Hz). ¹³C (133 MHz, CDCl₃): 199.87, 171.77, 158.33, 147.05, 135.86, 133.01, 127.58, 126.72, 114.77, 114.60, 66.36, 63.49, 55.48, 54.90, 44.57, 23.07, 14.77. MS (ESI) (M+H)⁺=437.500

### 1-[(Allyloxy)carbonyl]-5-(4-methoxyphenyl)-1,2,3,4,4a,5,6,10b-octahydrobenzo[h]-1,6-naphthyridine-9-carboxylic acid

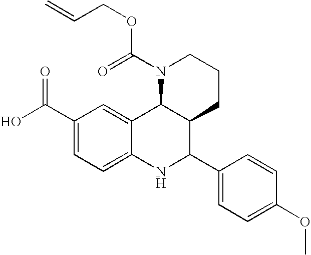 The title compound (3.15 g; yield: 95.0%) was prepared by following the general procedure 3.

¹H NMR (400 MHz, CDCl₃): 8.28 (0.4H, m), 8.18 (m, 0.6H), 7.77 (0.4H, dd, J=8.2, 0.4 Hz), 7.74 (0.6H, dd, J=8.2, 0.6 Hz), 7.35 (1H, d, J=8.2 Hz), 7.14 (1H,d, J=8.5 Hz), 6.83 (1H, d, J=8.6 Hz), 6.57 (1H, dd, J=8.6, 2.3 Hz), 6.00 (1H,m), 5.37 (1H,m), 5.27 (0.4H, m), 5.23 (0.6H, d, J=10.5 Hz), 4.87 (1H, m), 4.68 (1H,d, J=4.5 Hz), 4.59 (0.4H, dd, J=12.1, 4.3 Hz), 4.44 (0.4, d, J=1.9 Hz), 3.83 (1.8H, s), 3.77 (1.2H, s), 2.51 (1H, m), 2.08 (1.4H, m), 1.62 (0.6H, m). MS (ESI) (M+H)⁺=423.5.

### 1-[(Allyloxy)carbonyl]-5-phenyl-1,2,3,4,4a,5,6,10b-octahydrobenzo[h]-1,6-naphthyridine-9-carboxylic acid

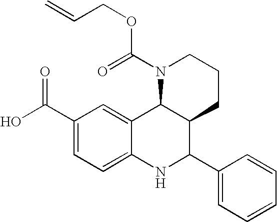 The titled compound (1.46 g; yield, 75%) was prepared by following the general procedure 3.

¹H NMR (400 MHz, CDCl₃, ppm): 8.23 (0.5H, m), 8.15 (0.5H, m), 7.78 (0.50H, dd, J=8.2, 1.6 Hz), 7.76 (0.50H, dd, J=8.2, 1.6 Hz), 7.43 (m, 2H), 7.28 (4H, m), 6.60 (1H, d, J=8.6 Hz), 5.95 (1H, m), 5.40 (1H,m), 5.33 (1.5H, m), 5.22 (0.5H, dd, J=10.3, 1.2 Hz), 4.85 (0.5H, m), 4.81 (1H,m), 4.66 (1H,m), 4.57 (1H, m), 4.49 (0.5H, d, J=2.0 Hz), 2.58 (1H,m), 2.17 (0.5H, m), 2.06 (1H, m), 1.56 (0.5H, m).

¹³C NMR (133 MHz, CDCl₃, ppm): 199.55, 155.50, 144.08, 132.63, 321.44, 130.57, 130.14, 128.64, 128.55, 127.26, 126.28, 125.38, 117.12, 113.97, 112.69, 66.26, 65.92, 56.36, 55.64, 54.90, 52.49, 49.14, 48.94, 48.72, 44.87, 44.71, 44.60, 43.66, 22.89.

MS (ESI) (M+H)⁺=393.4.

### 1-[(Allyloxy)carbonyl]-5-ethyl-4-phenyl-2,3,3a,4,5,9b-hexahydro-1H-pyrrolo[3,2-c]quinoline-8-carboxylic acid

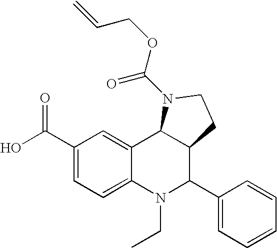 The titled compound (9.0 g; yield, 84%) was obtained by following the general procedure 4. MS (ESI) (M+H)⁺=407.474.

### Example 2

The titled compounds of Example 2 are made using the titled compounds made in Example 1 as the starting materials.

### tert-Butyl 8-[(4-methylpiperazin-1-yl)carbonyl]-4-phenyl-2,3,3a,4,5,9b-hexahydro-1H-pyrrolo[3,2-c]quinoline-1-carboxylate

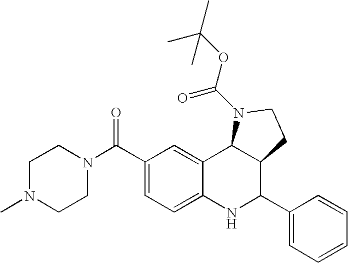 The titled compound (235.1 mg, 97% yield) was obtained by following the general procedure 5. (ESI) (M+H)⁺=477.6.

### tert-Butyl 8-(morpholin-4-ylcarbonyl)-4-phenyl-2,3,3a,4,5,9b-hexahydro-1H-pyrrolo[3,2-c]quinoline-1-carboxylate

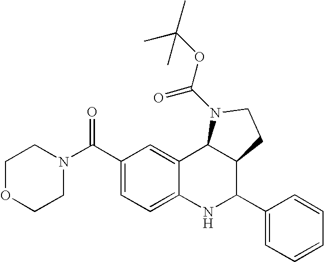 The titled compound (235.1 mg, 97% yield) was obtained by following the general procedure 5. (ESI) (M+H)⁺=464.6.

### tert-Butyl 4-phenyl-8-(pyrrolidin-1-ylcarbonyl)-2,3,3a,4,5,9b-hexahydro-1H-pyrrolo[3,2-c]quinoline-1-carboxylate

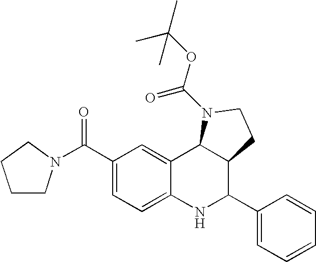 The titled compound (225.6 mg, 99% yield) was obtained by following the general procedure 5. (ESI) (M+H)⁺=448.6.

### tert-Butyl 8-{[(cyclopropylmethyl)amino]carbonyl}-4-phenyl-2,3,3a,4,5,9b-hexahydro-1H -pyrrolo[3,2-c]quinoline-1-carboxylate

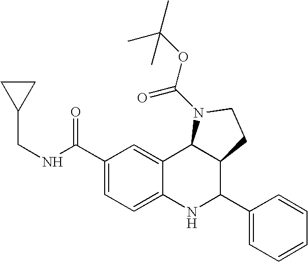 The titled compound (232.1 mg, 100% yield) was obtained by following the general procedure 5. (ESI) (M+H)⁺=448.6.

### tert-Butyl 4-phenyl-8-{[(tetrahydrofuran-2-ylmethyl)amino]carbonyl}-2,3,3a,4,5,9b-hexahydro-1H -pyrrolo[3,2-c]quinoline-1-carboxylate

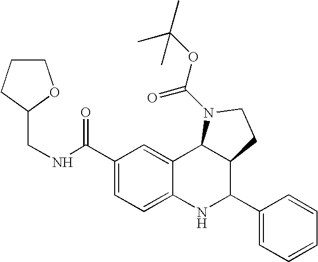 The titled compound (222.2 mg, 91.5% yield) was obtained by following the general procedure 5. (ESI) (M+H)⁺=478.6.

### tert-Butyl 8-{[(2-methoxyethyl)amino]carbonyl}-4-phenyl-2,3,3a,4,5,9b-hexahydro-1H -pyrrolo[3,2-c]quinoline-1-carboxylate

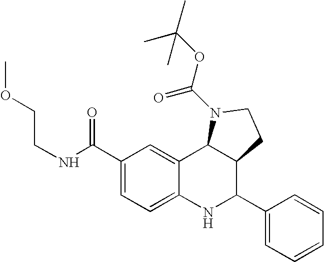 The titled compound (238.1 mg, 100% yield) was obtained by following the general procedure 5. (ESI) (M+H)⁺=452.5.

### tert-Butyl 8-({[2-(diethylamino)ethyl]amino}carbonyl)-4-phenyl-2,3,3a,4,5,9b-hexahydro-1H -pyrrolo[3,2-c]quinoline-1-carboxylate

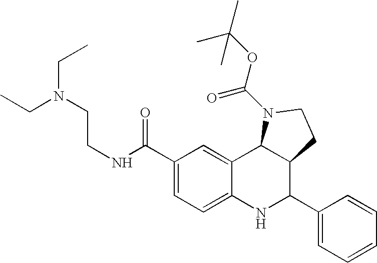 The titled compound (250.1 mg, 100% yield) was obtained by following the general procedure 5. (ESI) (M+H)⁺=493.6.

### tert-Butyl 8-[(diethylamino)carbonyl]-4-phenyl-2,3,3a,4,5,9b-hexahydro-1H-pyrrolo[3,2-c]quinoline-1-carboxylate

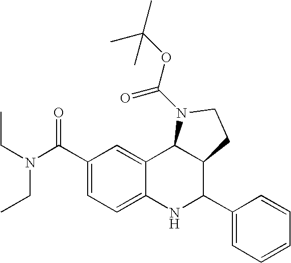 The titled compound (181.6 mg, 80% yield) was obtained by following the general procedure 5. (ESI) (M+H)⁺=450.6.

### tert-Butyl 4-(4-ethoxyphenyl)-8-[(4-methylpiperazin-1-yl)carbonyl]-2,3,3a,4,5,9b-hexahydro-1H-pyrrolo[3,2-c]quinoline-1-carboxylate

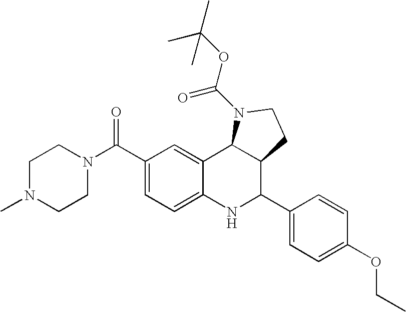 The titled compound (320 mg, 100% yield) was obtained by following the general procedure 5. (ESI) (M+H)⁺=521.7.

### tert-Butyl 4-(4-ethoxyphenyl)-8-(morpholin-4-ylcarbonyl)-2,3,3a,4,5,9b-hexahydro-1H -pyrrolo[3,2-c]quinoline-1-carboxylate

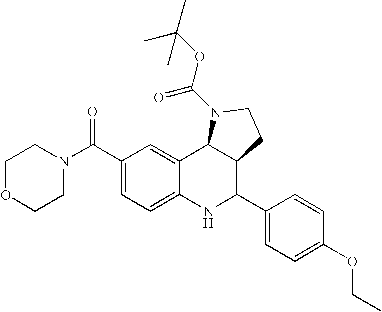 The titled compound (308 mg, 100% yield) was obtained by following the general procedure 5. (ESI) (M+H)⁺=508.6.

### tert-Butyl 4-(4-ethoxyphenyl)-8-(pyrrolidin-1-ylcarbonyl)-2,3,3a,4,5,9b-hexahydro-1H -pyrrolo[3,2-c]quinoline-1-carboxylate

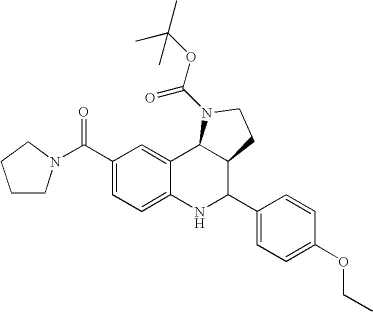 The titled compound (225.6 mg, 99% yield) was obtained by following the general procedure 5. (ESI) (M+H)⁺=492.6.

### tert-Butyl 8-{[(cyclopropylmethyl)amino]carbonyl}-4-(4-ethoxyphenyl)-2,3,3a,4,5,9b -hexahydro-1H-pyrrolo[3,2-c]quinoline-1-carboxylate

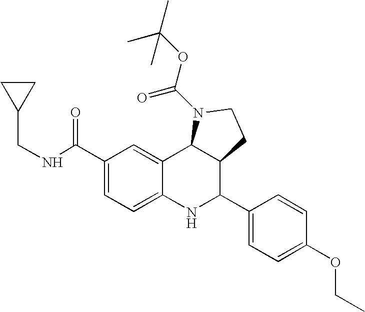 The titled compound (302.1 mg, 100% yield) was obtained by following the general procedure 5. (ESI) (M+H)⁺=492.6.

### tert-Butyl 4-(4-ethoxyphenyl)-8-{[(2-furylmethyl)(methyl)amino]carbonyl}-2,3,3a,4,5,9b -hexahydro-1H-pyrrolo[3,2-c]quinoline-1-carboxylate

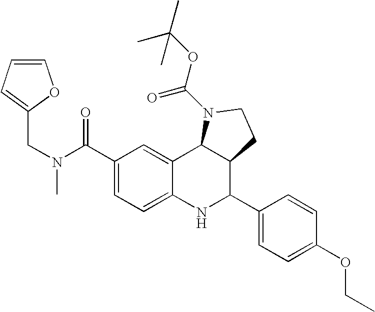 The titled compound (325 mg, 100% yield) was obtained by following the general procedure 5. (ESI) (M+H)⁺=532.6.

### tert-Butyl 4-(4-ethoxyphenyl)-8-{[(2-methoxyethyl)amino]carbonyl}-2,3,3a,4,5,9b-hexahydro-1H -pyrrolo[3,2-c]quinoline-1-carboxylate

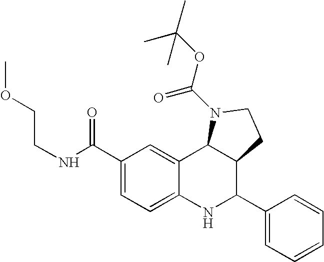 The titled compound (253.7 mg, 84% yield) was obtained by following the general procedure 5. (ESI) (M+H)⁺=496.6.

### tert-Butyl 8-({[2-(diethylamino)ethyl]amino}carbonyl)-4-(4-ethoxyphenyl)-2,3,3a,4,5,9b -hexahydro-1H-pyrrolo[3,2-c]quinoline-1-carboxylate

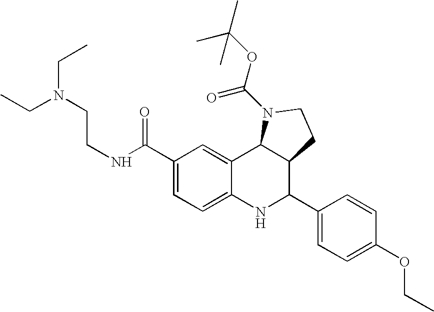 The titled compound (330 mg, 100% yield) was obtained by following the general procedure 5. (ESI) (M+H)⁺=537.7.

### tert-Butyl 8-[(diethylamino)carbonyl]-4-(4-ethoxyphenyl)-2,3,3a,4,5,9b-hexahydro-1H-pyrrolo[3,2-c]quinoline-1-carboxylate

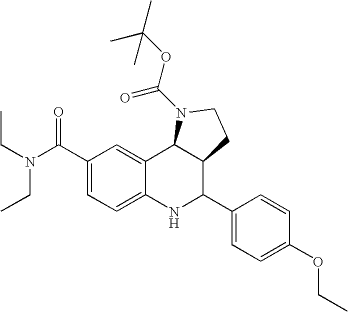 The titled compound (300 mg, 100% yield) was obtained by following the general procedure 5. (ESI) (M+H)⁺=494.6.

### Example 3

The titled compounds of Example 3 are made using the titled compounds made in Example 2 as the starting materials using one or more of the procedures described below.

### 8-[(4-Methylpiperazin-1-yl)carbonyl]-4-phenyl-2,3,3a,4,5,9b-hexahydro-1H-pyrrolo[3,2-c]quinoline

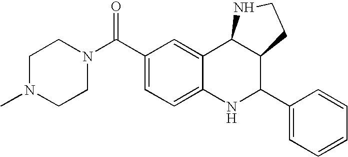 The titled compound (344.9 mg, 81% yield) was obtained by following the general procedure 7. (ESI) (M+H)⁺=377.5.

### 8-(Morpholin-4-ylcarbonyl)-4-phenyl-2,3,3a,4,5,9b-hexahydro-1H-pyrrolo[3,2-c]quinoline

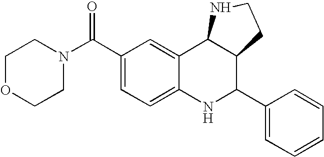 The titled compound (287.7 mg, 90% yield) was obtained by following the general procedure 7. (ESI) (M+H)⁺=364.4.

### 4-Phenyl-8-(pyrrolidin-1-ylcarbonyl)-2,3,3a,4,5,9b-hexahydro-1H-pyrrolo[3,2-c]quinoline

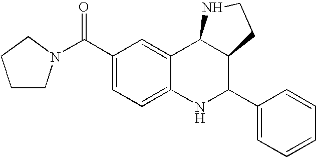 The titled compound (312 mg, 100% yield) was obtained by following the general procedure 7

(ESI) (M+H)⁺=348.4.

### N-(Cyclopropylmethyl)-4-phenyl-2,3,3a,4,5,9b-hexahydro-1H-pyrrolo[3,2-c]quinoline-8-carboxamide

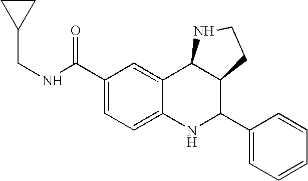 The titled compound (319.3 mg, 88% yield) was obtained by following the general procedure 7

(ESI) (M+H)⁺=348.4.

### 4-Phenyl-N-(tetrahydrofuran-2-ylmethyl)-2,3,3a,4,5,9b-hexahydro-1H-pyrrolo[3,2-c]quinoline-8-carboxamide

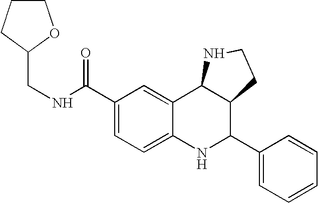 The titled compound (320 mg, 100% yield) was obtained by following the general procedure 7

(ESI) (M+H)⁺=378.5.

### N-(2-Methoxyethyl)-4-phenyl-2,3,3a,4,5,9b-hexahydro-1H-pyrrolo[3,2-c]quinoline-8-carboxamide

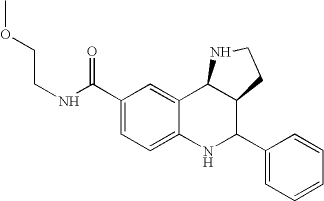 The titled compound (359.3 mg, 100% yield) was obtained by following the general procedure 7

(ESI) (M+H)⁺=352.4.

### N-[2-(Diethylamino)ethyl]-4-phenyl-2,3,3a,4,5,9b-hexahydro-1H-pyrrolo[3,2-c]quinoline-8-carboxamide

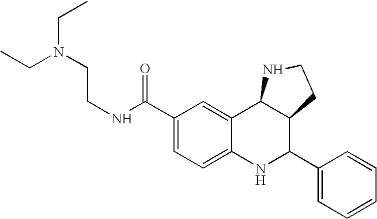 The titled compound (420.7 mg, 100% yield) was obtained by following the general procedure 7

(ESI) (M+H)⁺=393.5.

### N,N-Diethyl-4-phenyl-2,3,3a,4,5,9b-hexahydro-1H-pyrrolo[3,2-c]quinoline-8-carboxamide

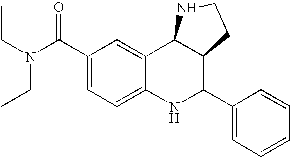 The titled compound (295.1 mg, 100% yield) was obtained by following the general procedure 7.

(ESI) (M+H)+350.5.

### 4-(4-Ethoxyphenyl)-8-[(4-methylpiperazin-1-yl)carbonyl]-2,3,3a,4,5,9b-hexahydro-1H-pyrrolo[3,2-c]quinoline

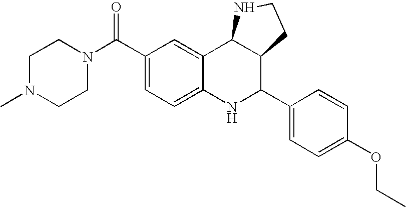 The titled compound (420.9 mg, 100% yield) was obtained by following the general procedure 7.

(ESI) (M+H)⁺=421.5.

### 4-(4-Ethoxyphenyl)-8-(morpholin-4-ylcarbonyl)-2,3,3a,4,5,9b-hexahydro-1H-pyrrolo[3,2-c]quinoline

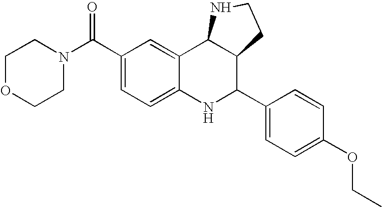 The titled compound (290.6 mg, 90% yield) was obtained by following the general procedure 7.

(ESI) (M+H)⁺=408.5.

### 4-(4-Ethoxyphenyl)-8-(pyrrolidin-1-ylcarbonyl)-2,3,3a,4,5,9b-hexahydro-1H-pyrrolo[3,2-c]quinoline

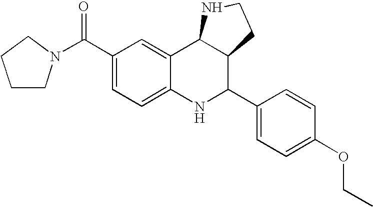 The titled compound (394.3 mg, 100% yield) was obtained by following the general procedure 7

(ESI) (M+H)⁺=392.5.

### N-(Cyclopropylmethyl)-4-(4-ethoxyphenyl)-2,3,3a,4,5,9b-hexahydro-1H-pyrrolo[3,2-c]quinoline-8-carboxamide

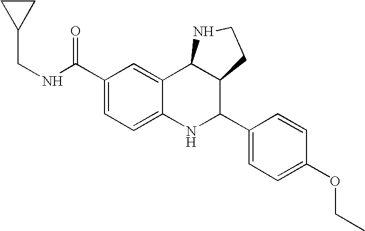 The titled compound (325.3 mg, 88% yield) was obtained by following the general procedure 7

(ESI) (M+H)⁺=392.5.

### 4-(4-Ethoxyphenyl)-N-(2-furylmethyl)-N-methyl-2,3,3a,4,5,9b-hexahydro-1H-pyrrolo[3,2-c]quinoline-8-carboxamide

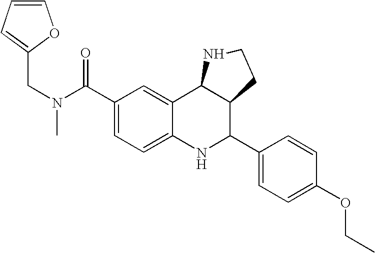 The titled compound (325 mg, 100% yield) was obtained by following the general procedure 7

(ESI) (M+H)⁺=432.5.

### N-(2-Methoxyethyl)-4-phenyl-2,3,3a,4,5,9b-hexahydro-1H-pyrrolo[3,2-c]quinoline-8-carboxamide

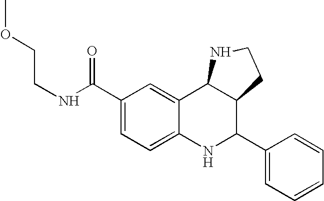 The titled compound (330.3 mg, 90% yield) was obtained by following the general procedure 7

(ESI) (M+H)⁺=352.4.

### N-[2-(Diethylamino)ethyl]-4-(4-ethoxyphenyl)-2,3,3a,4,5,9b-hexahydro-1H-pyrrolo[3,2-c]quinoline-8-carboxamide

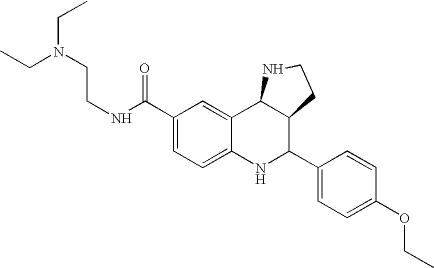 The titled compound (340.6 mg, 80% yield) was obtained by following the general procedure 7

(ESI) (M+H)⁺=437.6.

### (4-(4-Ethoxyphenyl)-N,N-diethyl-2,3,3a,4,5,9b-hexahydro-1H-pyrrolo[3,2-c]quinoline-8-carboxamide

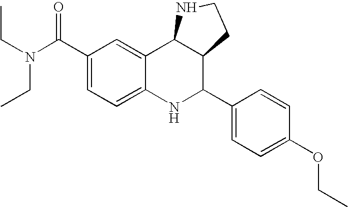 The titled compound (155.7 mg, 60% yield) was obtained by following the general procedure 7

(ESI) (M+H)⁺=394.5.

### Example 4

The titled compounds of Example 3 are reacted with the R⁵COCl listed below in a parallel format in a 2 mL deep 96-well microtiter plate to form the compounds of the present invention using General Procedure 12 below.

General Procedure 12 (amide formation) 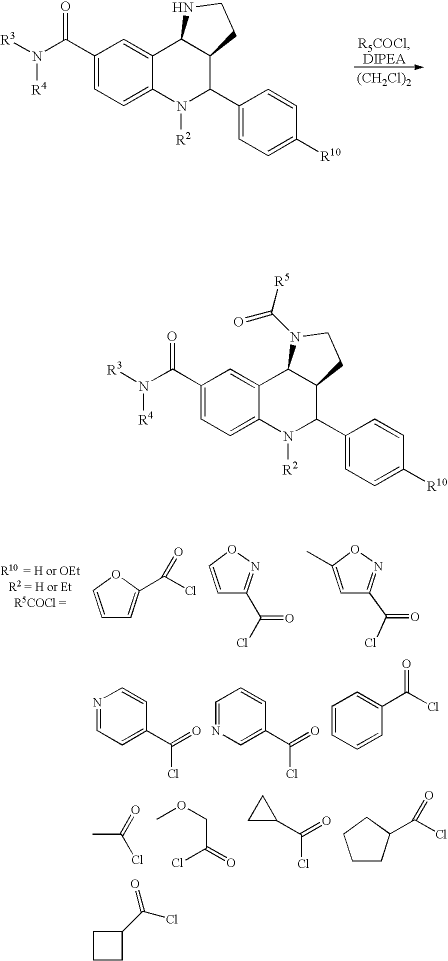
To the amine (˜20 μmol/well, 1 equiv.) and DIPEA (5 equiv.) in (CH₂Cl)₂ (300 μl/well) was added acyl chloride (2 equiv.). The 96-well microtiter plate was then shaken for 20 hours at 40⁰C. Then, the reaction solution was diluted with CH₂Cl₂ (1 mL). The excess amount of reagents were quenched with 5% aqueous NaOH (400 μl/well). The plate was shaken for another 30 minutes. Afterwards, the solutions were passed through hydromatrix (2 mL/well) and the collected filtrates were evaporated in In vacuo to give the products.

### Example 5

The titled compounds of Example 3 are reacted with the R⁶SO₂Cl listed below in a 96-well plate format to form the compounds of the present invention using General Procedure 13 below.

General Procedure 13 (sulphonyl amide formation) 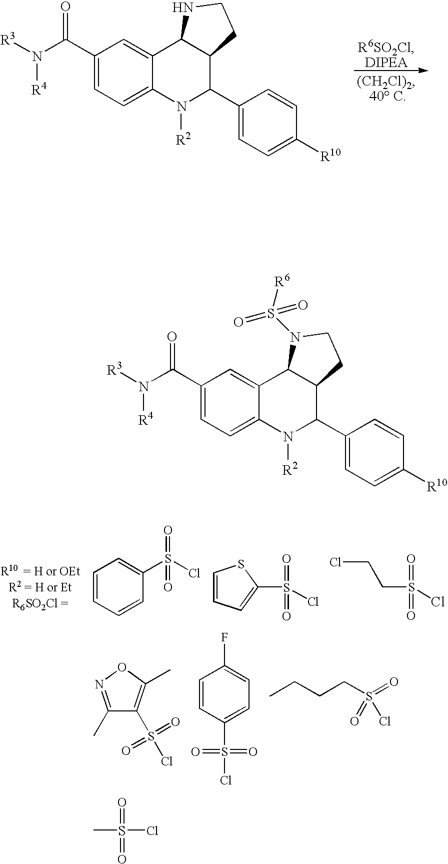
To the amine (˜20 μmol/well, 1 equiv.) and DIPEA (5 equiv.) in (CH₂Cl)₂ (300 μl/well) was added sulfonyl chloride (3 equiv.). The 96-well plate was shaken for 20 hours at 40⁰C. Then, the reaction solution was diluted with CH₂Cl₂ (1 mL). The excess amount of reagents were quenched with 5% aqueous NaOH (500 μl/well). The plate was shaken for another 30 minutes. Afterwards, the solutions were passed through hydromatrix (2 mL/well) and the filtrates were evaporated in In vacuo to give the products.

### Example 6

The titled compounds of Example 3 are reacted with the R⁷NCX listed below in plate format to form the compounds of the present invention using General Procedure 14 below.

General Procedure 14 (urea or thiourea formation): 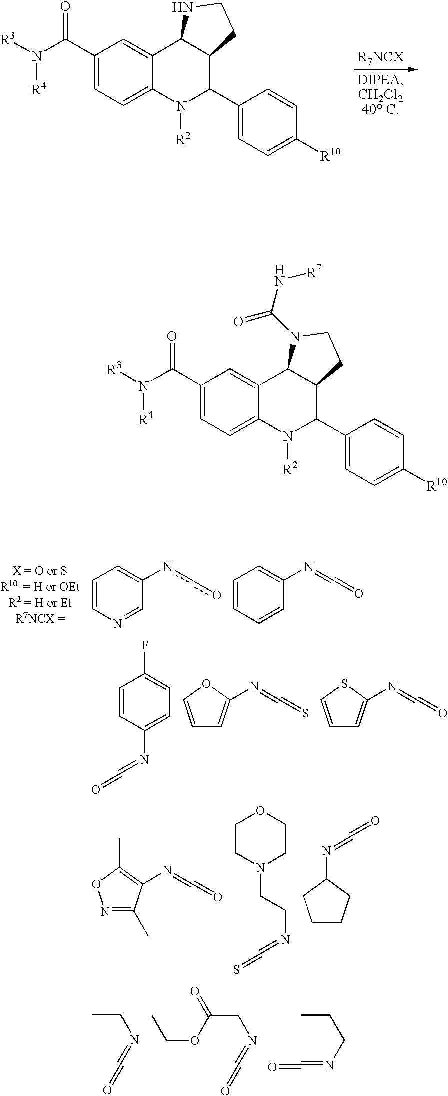
To the amine (1 equiv.) and DIPEA (3 equiv.) in (CH₂Cl)₂ was added isocyanate or thioisocyanate (3 equiv.). The plate was shaken for eight hours at 40⁰C. The scavenger resin (5 equiv.), aminomethyl polystyrene resin, was added to each well. The plate was shaken for another 30 minutes. Then, the solutions were filtered and the resin was washed with DCM. The combined solvents in plate were evaporated in In vacuo to give the products.

### Example 7

The titled compounds of Example 3 are reacted with the R⁸CHO listed below in plate format to form the compounds of the present invention using General Procedure 15 below.

General Procedure 15 (Reductive amination) 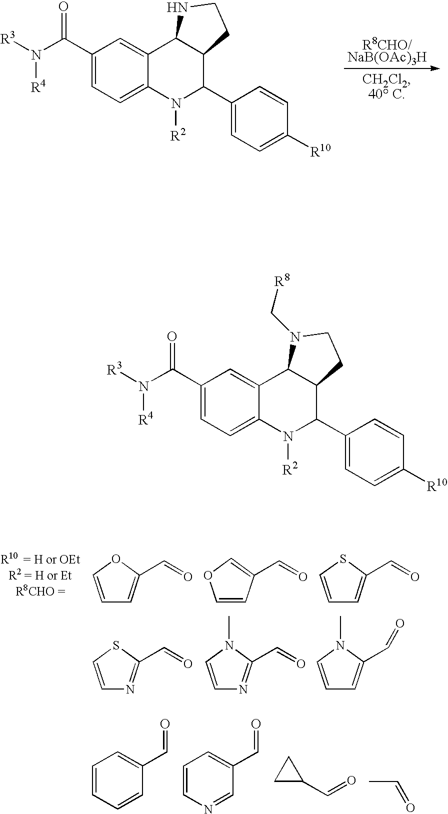
To the amine (˜20 μmol/well, 1 equiv.), NaBH(OAc)₃ (1.5 equiv.) and HOAc (5 equiv.) in (CH₂Cl)₂ (300 μl/well) added the aldehyde (1.5 equiv.). The plate was then shaken for 5 hours at 40⁰C. Then, the reaction solutions were diluted with CH₂Cl₂ (1 mL). The solutions were quenched with 5% aqueous NaOH (500 μl/well). The plate was shaken for another 30 minutes. Afterwards, the solutions were passed through hydromatrix (2 mL/well) and the filtrates were evaporated in In vacuo to give the products.
In EXAMPLES 4-7, 960 compounds (12 plates) were prepared. As a standard procedure, 10 out of every 80 compounds were checked for purity. The purity analysis was performed by analytical LCMS (UV detection). The purity check showed that 75% of selected compounds have purity over 50%. The estimated material in each well was 10-17 mg.

### Example 8

### 1H-Pyrrolo[3,2-c]quinoline-1-carboxylic acid, 8-[[[(1-ethyl-2-pyrrolidinyl)methyl]amino]carbonyl]-2,3,3a,4,5,9b-hexahydro-4-phenyl-, 2-propenyl ester

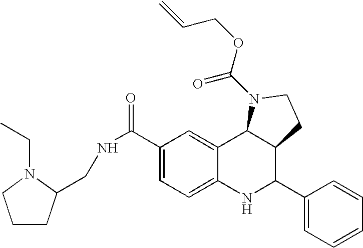 The titled compound (90.6 mg, 99% yield) was obtained by following the general procedure 5

(ESI) (M+H)⁺=489.6.

### 1H-Pyrrolo[3,2-c]quinoline-1-carboxylic acid, 8-[[[2-(1-ethyl-2-pyrrolidinyl)ethyl]amino]carbonyl]-2,3,3a,4,5,9b-hexahydro-4-(4-methoxyphenyl)-, 2-propenyl ester

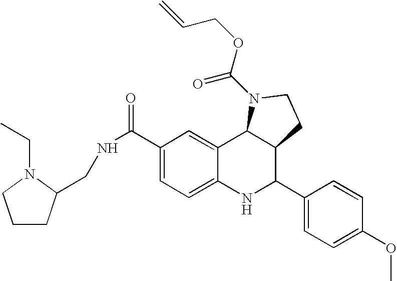 The titled compound (76.4 mg, 78.5% yield) was obtained by following the general procedure 5

(ESI) (M+H)⁺=519.6.

### 1H-Pyrrolo[3,2-c]quinoline-1-carboxylic acid, 8-[[[(1-ethyl-2-pyrrolidinyl)methyl]amino]carbonyl]-2,3,3a,4,5,9b-hexahydro-4-(2-pyridinyl)-, 2-propenyl ester

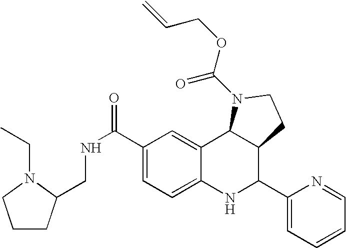 The titled compound (83.3 mg, 83%) was obtained by following the general procedure 5

(ESI) (M+H)⁺=490.6.

### 1H-Pyrrolo[3,2-c]quinoline-1-carboxylic acid, 2,3,3a,4,5,9b-hexahydro-8-[(4-methyl-1-piperazinyl)carbonyl]-4-phenyl-, 2-propenyl ester

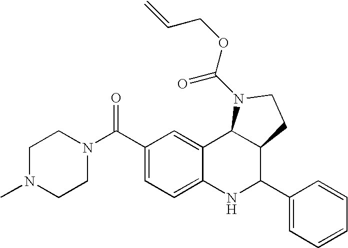 The titled compound (86.4 mg, 100%) was prepared by following the general procedure 5

(ESI) (M+H)⁺=461.568.

### 1H-Pyrrolo[3,2-c]quinoline-1-carboxylic acid, 2,3,3a,4,5,9b-hexahydro-4-(4-methoxyphenyl)-8-[(4-methyl-1-piperazinyl)carbonyl]-, 2-propenyl ester

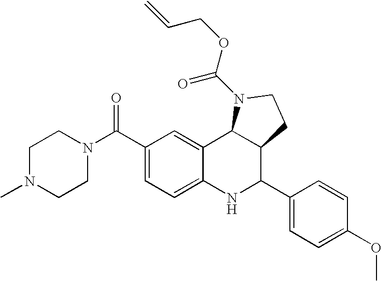 The titled compound (75.2 mg, 82%) was prepared by following the general procedure 5

(ESI) (M+H)⁺=490.6.

### 1H-Pyrrolo[3,2-c]quinoline-1-carboxylic acid, 2,3,3a,4,5,9b-hexahydro-8-[(4-methyl-1-piperazinyl)carbonyl]-4-(2-pyridinyl)-, 2-propenyl ester

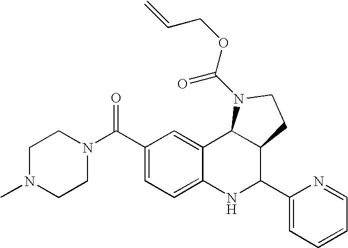 The titled compound (81.2 mg, 94%) was obtained by following the general procedure 5

(ESI) (M+H)⁺=462.6.

### 1H-Pyrrolo[3,2-c]quinoline-1-carboxylic acid, 8-[[[2-(diethylamino)ethyl]amino]carbonyl]-2,3,3a,4,5,9b-hexahydro-4-phenyl-, 2-propenyl ester

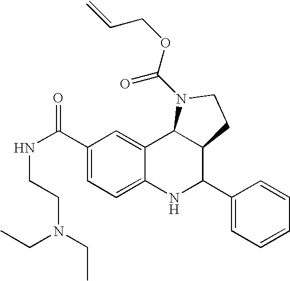 The titled compound (89.9 mg, 100%) was obtained by following the general procedure 5

(ESI) (M+H)⁺=477.611.

### 1H-Pyrrolo[3,2-c]quinoline-1-carboxylic acid, 8-[[[2-(diethylamino)ethyl]amino]carbonyl]-2,3,3a,4,5,9b-hexahydro-4-(4-methoxyphenyl)-, 2-propenyl ester

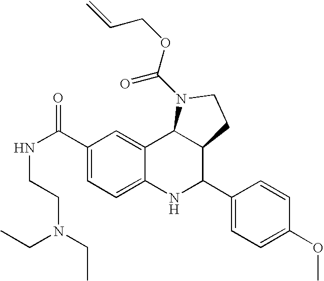 The titled compound (84.3 mg, 89%) was obtained by following the general procedure 5

(ESI) (M+H)⁺=507.6.

### 1H-Pyrrolo[3,2-c]quinoline-1-carboxylic acid, 8-[[[2-(diethylamino)ethyl]amino]carbonyl]-2,3,3a,4,5,9b-hexahydro-4-(2-pyridinyl)-, 2-propenyl ester

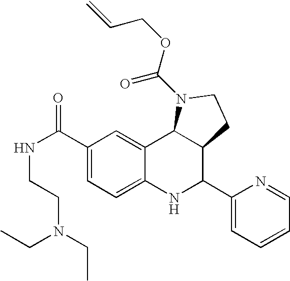 The titled compound (73.9 mg, 82%) was obtained by following the general procedure 5

(ESI) (M+H)⁺=478.6.

### 1H-Pyrrolo[3,2-c]quinoline-1-carboxylic acid, 2,3,3a,4,5,9b-hexahydro-4-(4-methoxyphenyl)-8-[[(2-pyridinylmethyl)amino]carbonyl]-, 2-propenyl ester

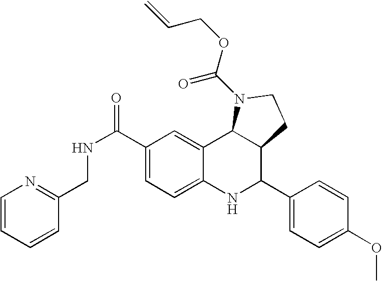 The titled compound (79.2 mg, 84%) was obtained by following the general procedure 5

(ESI) (M+H)⁺=499.6.

### 1H-Pyrrolo[3,2-c]quinoline-1-carboxylic acid, 2,3,3a,4,5,9b-hexahydro-4-phenyl-8-[[(2-pyridinylmethyl)amino]carbonyl]-, 2-propenyl ester

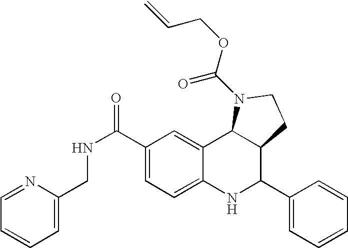 The titled compound (74.5 mg, 85%) was obtained by following the general procedure 5

(ESI) (M+H)⁺=469.547.

### 1H-Pyrrolo[3,2-c]quinoline-1-carboxylic acid, 2,3,3a,4,5,9b-hexahydro-4-(2-pyridinyl)-8-[[(2-pyridinylmethyl)amino]carbonyl]-, 2-propenyl ester

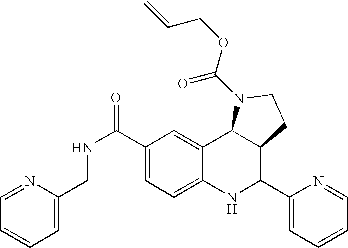 The titled compound (75.6 mg, 86%) was obtained by following the general procedure 5

(ESI) (M+H)⁺=470.5.

### 1H-Pyrrolo[3,2-c]quinoline-1-carboxylic acid, 8-[(4-formyl-1-piperazinyl)carbonyl]-2,3,3a,4,5,9b -hexahydro-4-phenyl-, 2-propenyl ester

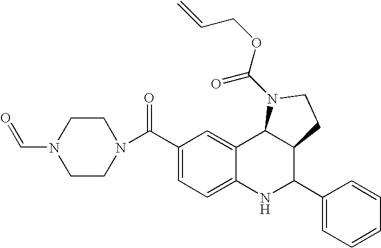 The titled compound (81 mg, 100%) was obtained by following the general procedure 5

(ESI) (M+H)+475.6.

### 1H-Pyrrolo[3,2-c]quinoline-1-carboxylic acid, 8-[(4-formyl-1-piperazinyl)carbonyl]-2,3,3a,4,5,9b -hexahydro-4-(2-pyridinyl)-, 2-propenyl ester

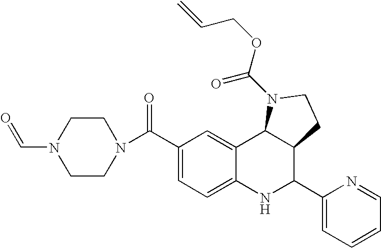 The titled compound (78.8 mg, 97%) was obtained by following the general procedure 5

(ESI) (M+H)⁺=476.5.

### 1H-Pyrrolo[3,2-c]quinoline-1-carboxylic acid, 2,3,3a,4,5,9b-hexahydro-4-phenyl-8-[[4-(phenylmethyl)-1-piperazinyl]carbonyl]-, 2-propenyl ester

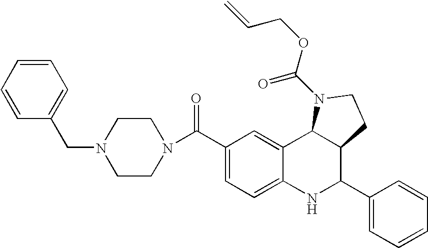 The titled compound (90.2 mg, 99%) was obtained by following the general procedure 5

(ESI) (M+H)⁺=537.7.

### 1H-Pyrrolo[3,2-c]quinoline-1-carboxylic acid, 2,3,3a,4,5,9b-hexahydro-8-[[4-(phenylmethyl)-1-piperazinyl]carbonyl]-4-(2-pyridinyl)-, 2-propenyl ester

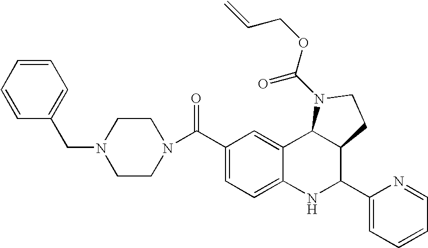 The titled compound (89.4 mg, 98%) was obtained by following the general procedure 5

(ESI) (M+H)⁺=538.7.

### 1H-Pyrrolo[3,2-c]quinoline-1-carboxylic acid, 8-[[[2-[bis(1-methylethyl)amino]ethyl]amino]carbonyl]-2,3,3a,4,5,9b-hexahydro-4-phenyl-, 2-propenyl ester

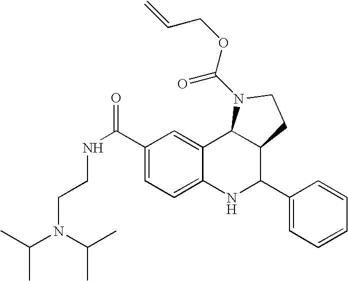 The titled compound (80.5 mg, 94%) was obtained by following the general procedure 5

(ESI) (M+H)⁺=505.7.

### 1H-Pyrrolo[3,2-c]quinoline-1-carboxylic acid, 8-[[[2-[bis(1-methylethyl)amino]ethyl]amino]carbonyl]-2,3,3a,4,5,9b-hexahydro-4-(2-pyridinyl)-, 2-propenyl ester

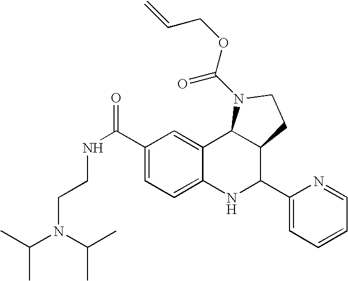 The titled compound (79.4 mg, 92%) was obtained by following the general procedure 5

(ESI) (M+H)⁺=506.7.

### 1H-Pyrrolo[3,2-c]quinoline-1-carboxylic acid, 8-[[[2-(dimethylamino)ethyl]amino]carbonyl]-2,3,3a,4,5,9b-hexahydro-4-phenyl-, 2-propenyl ester

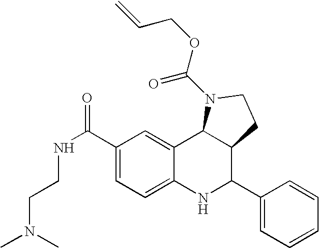 The titled compound (74.6 mg, 98%) was obtained by following the general procedure 5.

(ESI) (M+H)⁺=449.6.

### 1H-Pyrrolo[3,2-c]quinoline-1-carboxylic acid, 8-[[[2-(dimethylamino)ethyl]amino]carbonyl]-2,3,3a,4,5,9b-hexahydro-4-(2-pyridinyl)-, 2-propenyl ester

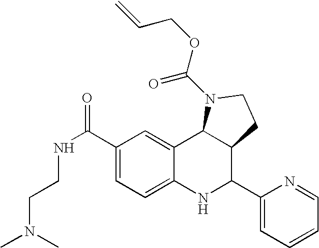 The titled compound (70.5 mg, 92%) was obtained by following the general procedure 5

(ESI) (M+H)⁺=450.5.

### 1H-Pyrrolo[3,2-c]quinoline-1-carboxylic acids 8-[[[2-(diethylamino)ethyl]methyl amino]carbonyl]-2,3,3a,4,5,9b-hexahydro-4-phenyl-, 2-propenyl ester

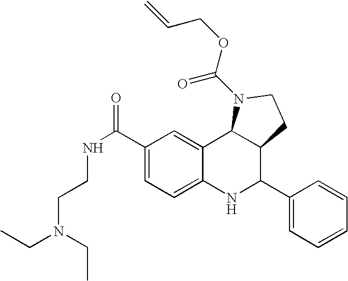 The titled compound (79.5 mg, 86%) was obtained by following the general procedure 5

(ESI) (M+H)⁺=491.6.

### 1H-Pyrrolo[3,2-c]quinoline-1-carboxylic acid, 8-[[[2-(diethylamino)ethyl]methylamino]carbonyl]-2,3,3a,4,5,9b-hexahydro-4-(2-pyridinyl)-, 2-propenyl ester

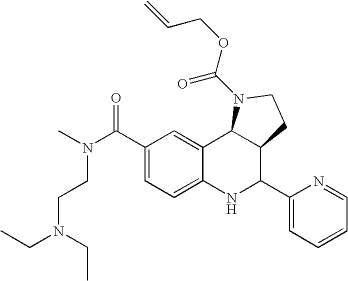 The titled compound (75.3 mg, 92%) was obtained by following the general procedure 5

(ESI) (M+H)⁺=492.6.

### 1H-Pyrrolo[3,2-c]quinoline-1-carboxylic acid, 2,3,3a,4,5,9b-hexahydro-4-phenyl-8-[[[2-(4-thiomorpholinyl)ethyl]amino]carbonyl]-, 2-propenyl ester

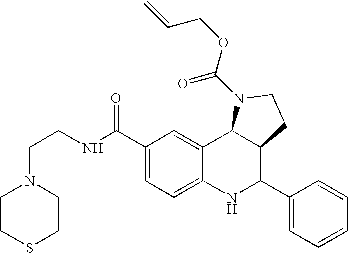 The titled compound (76.4 mg, 89%) was obtained by following the general procedure 5

(ESI) (M+H)⁺=507.7.

### 1H-Pyrrolo[3,2-c]quinoline-1-carboxylic acid, 2,3,3a,4,5,9b-hexahydro-4-(2-pyridinyl)-8-[[[2-(4-thiomorpholinyl)ethyl]amino]carbonyl]-, 2-propenyl ester

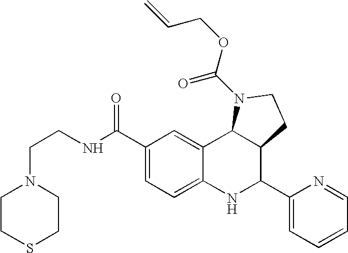 The titled compound (67.4 mg, 88%) was obtained by following the general procedure 5

(ESI) (M+H)⁺=508.6.

### Example 9

The titled compounds of Example 9 are made using the titled compounds made in Example 8 as the starting materials.

### N-[2-(Diethylamino)ethyl]-4-phenyl-2,3,3a,4,5,9b-hexahydro-1H-pyrrolo[3,2-c]quinoline-8-carboxamide

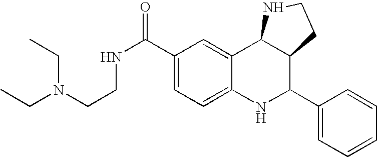 The titled compound (65.4 mg, 97.8% yield) was obtained by following the general procedure 6

(ESI) (M+H)⁺=393.5.

### Piperazine, 1-[(2,3,3a,4,5,9b-hexahydro-4-phenyl-1H-pyrrolo[3,2-c]quinolin-8-yl)carbonyl]-4-methyl-

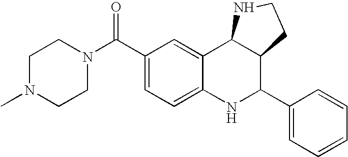 The titled compound (61.7 mg, 96%) was prepared by following the general procedure 6

(ESI) (M+H)⁺=377.5.

### Piperazine, 1-[[2,3,3a,4,5,9b-hexahydro-4-(4-methoxyphenyl)-1H-pyrrolo[3,2-c]quinolin-8-yl]carbonyl]-4-methyl-

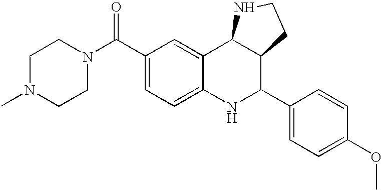 The titled compound (65.7 mg, yield, 86%) was prepared by following the general procedure 6

(ESI) (M+H)⁺=407.5.

### Piperazine, 1-[[2,3,3a,4,5,9b-hexahydro-4-(2-pyridinyl)-1H-pyrrolo[3,2-c]quinolin-8-yl]carbonyl]-4-methyl-

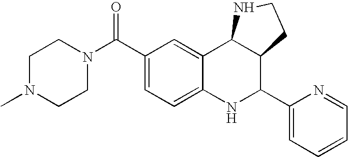 The titled compound (67.5 mg, yield, 94%) was obtained by following the general procedure 6

(ESI) (M+H)⁺=378.5.

### 1H-Pyrrolo[3,2-c]quinoline-8-carboxamide, N-[(1-ethyl-2-pyrrolidinyl)methyl]-2,3,3a,4,5,9b -hexahydro-4-phenyl-

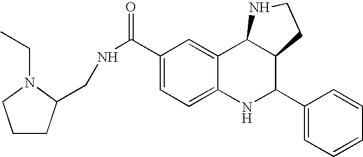 The titled compound (67.1 mg, yield, 88%) was obtained by following the general procedure 6

(ESI) (M+H)⁺=405.5.

### 1H-Pyrrolo[3,2-c]quinoline-8-carboxamide, N-[2-(diethylamino)ethyl]-2,3,3a,4,5,9b-hexahydro-4-(4-methoxyphenyl)-

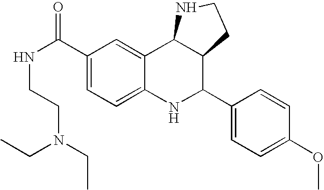 The titled compound (61.5 mg, yield, 78%) was obtained by following the general procedure 6

(ESI) (M+H)⁺=423.6.

### 1H-Pyrrolo[3,2-c]quinoline-8-carboxamide, N-[(1-ethyl-2-pyrrolidinyl)methyl]-2,3,3a,4,5,9b -hexahydro-4-(2-pyridinyl)-

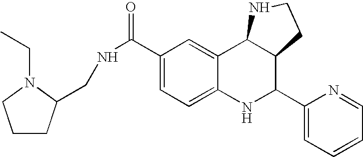 The titled compound (76.4 mg, yield, 100%) was obtained by following the general procedure 6

(ESI) (M+H)⁺=406.5.

### 1H-Pyrrolo[3,2-c]quinoline-8-carboxamide, N-[(1-ethyl-2-pyrrolidinyl)methyl]-2,3,3a,4,5,9b -hexahydro-4-(4-methoxyphenyl)-

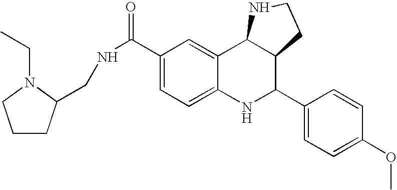 The titled compound (74.0 mg, yield, 91%) was obtained by following the general procedure 6

(ESI) (M+H)⁺=435.6.

### 1H-Pyrrolo[3,2-c]quinoline-8-carboxamide, 2,3,3a,4,5,9b-hexahydro-4-(4-methoxyphenyl)-N-(2-pyridinylmethyl)-

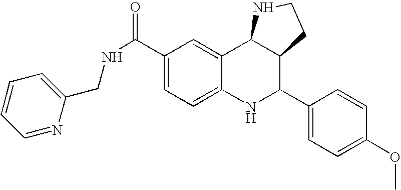 The titled compound (66.0 mg, yield, 85%) was obtained by following the general procedure 6

(ESI) (M+H)⁺=415.5.

### 1H-Pyrrolo[3,2-c]quinoline-8-carboxamide, 2,3,3a,4,5,9b-hexahydro-4-phenyl-N-(2-pyridinylmethyl)-

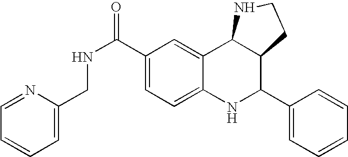 The titled compound (68.3 mg; yield, 95%) was obtained by following the general procedure 6

(ESI) (M+H)⁺=385.5.

### 1H-Pyrrolo[3,2-c]quinoline-8-carboxamide, 2,3,3a,4,5,9b-hexahydro-4-(2-pyridinyl)-N-(2-pyridinylmethyl)-

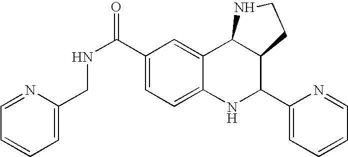 The titled compound (63.2; yield, 87%) was obtained by following the general procedure 6

(ESI) (M+H)⁺=386.5.

### 1H-Pyrrolo[3,2-c]quinoline-8-carboxamide, N-[2-(diethylamino)ethyl]-2,3,3a,4,5,9b-hexahydro-4-(2-pyridinyl)-

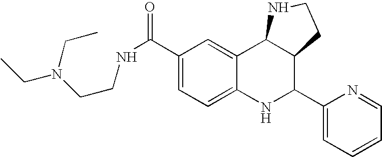 The titled compound (72.4; yield, 98%) was obtained by following the general procedure 6

(ESI) (M+H)⁺=394.5.

### 1-Piperazinecarboxaldehyde, 4-[(2,3,3a,4,5,9b-hexahydro-4-phenyl-1H-pyrrolo[3,2-c]quinolin-8-yl)carbonyl]-

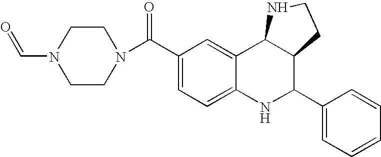 The titled compound (65.4 mg; yield, 89%) was obtained by following the general procedure 6

(ESI) (M+H)⁺=391.5.

### 1-Piperazinecarboxaldehyde, 4-[[2,3,3a,4,5,9b-hexahydro-4-(2-pyridinyl)-1H-pyrrolo[3,2-c]quinolin-8-yl]carbonyl]-

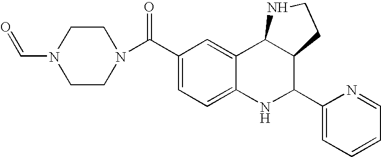 The titled compound (72.1 mg; yield, 98%) was obtained by following the general procedure 6

(ESI) (M+H)⁺=392.5.

### Piperazine, 1-[(2,3,3a,4,5,9b-hexahydro-4-phenyl-1H-pyrrolo[3,2-c]quinolin-8-yl)carbonyl]-4-(phenylmethyl)-

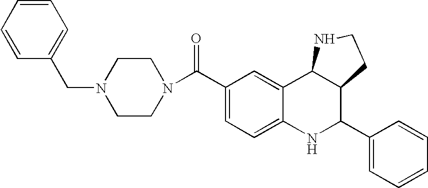 The titled compound (69.7 mg; yield, 82%) was obtained by following the general procedure 6

(ESI) (M+H)⁺=453.6.

### Piperazine, 1-[[2,3,3a,4,5,9b-hexahydro-4-(2-pyridinyl)-1H-pyrrolo[3,2-c]quinolin-8-yl]carbonyl]-4-(phenylmethyl)-

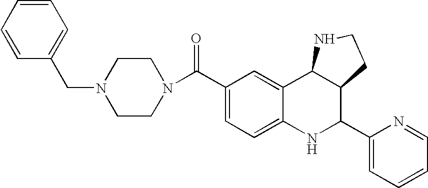 The titled compound (84.7 mg; yield, 100%) was obtained by following the general procedure 6

(ESI) (M+H)⁺=453.6.

### 1H-Pyrrolo[3,2-c]quinoline-8-carboxamide, N-[2-[bis(1-methylethyl amino]ethyl]-2,3,3a,4,5,9b -hexahydro-4-phenyl-

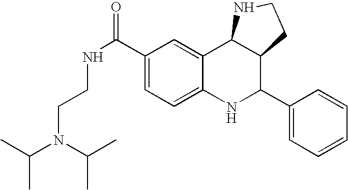 The titled compound (84.7 mg; yield, 100%) was obtained by following the general procedure 6

(ESI) (M+H)⁺=421.6.

### 1H-Pyrrolo[3,2-c]quinoline-8-carboxamide, N-[2-[bis(1-methylethyl)amino]ethyl]-2,3,3a,4,5,9b -hexahydro-4-(2-pyridinyl)-

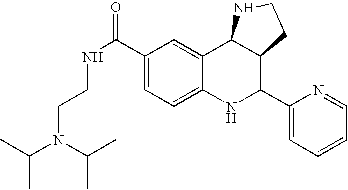 The titled compound (74.2 mg; yield, 94%) was obtained by following the general procedure 6

(ESI) (M+H)⁺=422.6.

### 1H-Pyrrolo[3,2-c]quinoline-8-carboxamide, N-[2-(dimethylamino)ethyl]-2,3,3a,4,5,9b -hexahydro-4-(2-pyridinyl)-

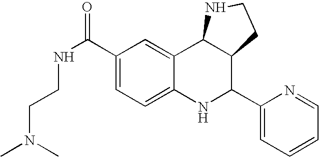 The titled compound (65.7 mg; yield, 96%) was obtained by following the general procedure 6

(ESI) (M+H)⁺=366.5.

### 1H-Pyrrolo[3,2-c]quinoline-8-carboxamide, N-[2-(dimethylamino)ethyl]-2,3,3a,4,5,9b -hexahydro-4-phenyl-

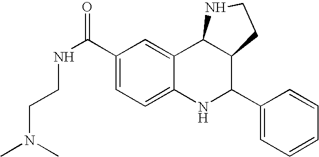 The titled compound (74.2 mg; yield, 100%) was obtained by following the general procedure 6

(ESI) (M+H)⁺=365.5.

### 1H-Pyrrolo[3,2-c]quinoline-8-carboxamide, N-[2-(diethylamino)ethyl]-2,3,3a,4,5,9b-hexahydro-N-methyl-4-phenyl-

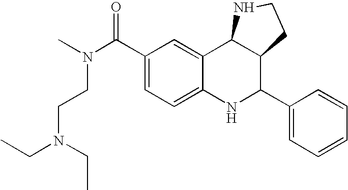 The titled compound (75.5 mg; yield, 99%) was obtained by following the general procedure 6

(ESI) (M+H)⁺=407.6.

### 1H-Pyrrolo[3,2-c]quinoline-8-carboxamide, N-[2-(diethylamino)ethyl]-2,3,3a,4,5,9b-hexahydro-N-methyl-4-(2-pyridinyl)-

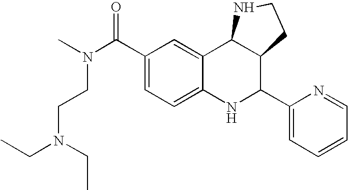 The titled compound (65.7 mg; yield, 86%) was obtained by following the general procedure 6

(ESI) (M+H)⁺=408.6.

### 1H-Pyrrolo[3,2-c]quinoline-8-carboxamide, 2,3,3a,4,5,9b-hexahydro-4-phenyl-N-[2-(4-thiomorpholinyl)ethyl]-

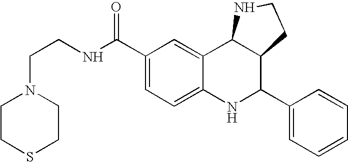 The titled compound (70.4 mg; yield, 89%) was obtained by following the general procedure 6

(ESI) (M+H)⁺=423.6.

### 1H-Pyrrolo[3,2-c]quinoline-8-carboxamide, 2,3,3a,4,5,9b-hexahydro-4-(2-pyridinyl)-N-[2-(4-thiomorpholinyl)ethyl]-

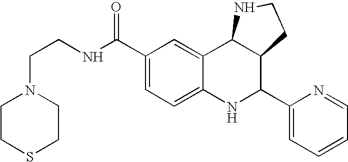 The titled compound (84.1 mg; yield, 100%) was obtained by following the general procedure 6

(ESI) (M+H)⁺=424.6.

### Example 10

The titled compounds of Example 9 are reacted with the R⁸CHO listed below in a 96-well plate format to form the compounds of the present invention using General Procedure 16 below.

General Procedure 16 (Reductive amination) 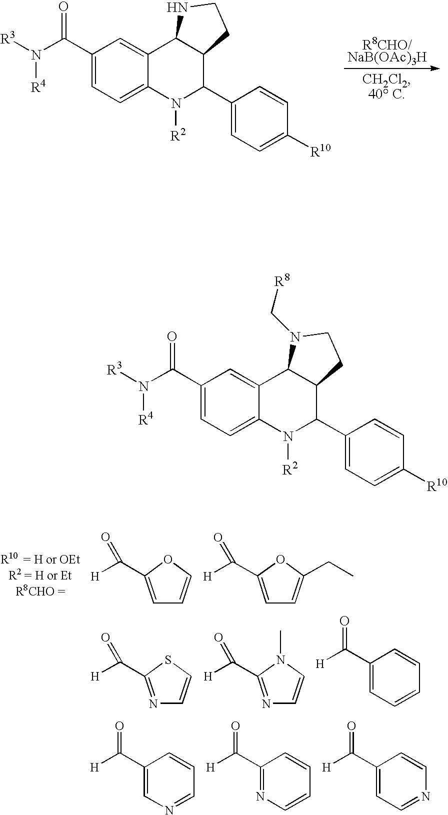
Following the general procedure 15 described before, 400 compounds (5 plates) were prepared. 10 out of 80 compounds were checked for purity. The purity analysis was performed by analytical LCMS (UV detection). The purity check showed that 85% of selected compounds have purity over 50%. The estimated material in each well is 10-15 mg.

### Example 11

### Allyl-5-(4-ethoxyphenyl)-9-(pyrrolidin-1-ylcarbonyl)-3,4,4a,5,6,10b-hexahydrobenzo[h]-1,6-naphthyridine-1(2H)-carboxylate

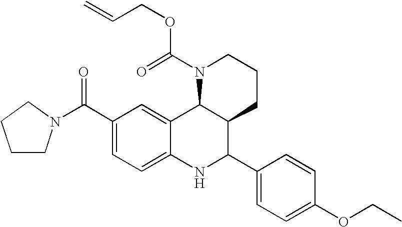 The titled compound (1.01 g; yield, 79%) was obtained by following the general procedure 5

(ESI) (M+H)⁺=490.6.

### Benzo[h][1,6]naphthyridine-1(2H)-carboxylic acid, 5-(4-ethoxyphenyl)-3,4,4a,5,6,10b-hexahydro-9-[[(2-methoxyethyl)amino]carbonyl]-, 2-propenyl ester

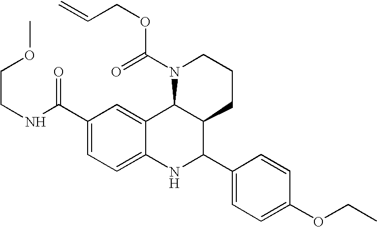 The titled compound (0.86 g; yield, 67%) was obtained by following the general procedure 5

(ESI) (M+H)⁺=494.6.

### Benzo[h][1,6]naphthyridine-1(2H)-carboxylic acid, 9-[(cyclopentylamino)carbonyl]-5-(4-ethoxyphenyl)-3,4,4a,5,6,10b-hexahydro-, 2-propenyl ester

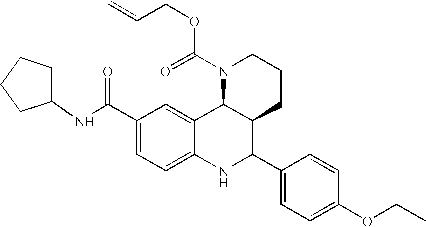 The titled compound (1.05 g; yield, 80%) was obtained by following the general procedure 5

(ESI) (M+H)⁺=504.6.

### Benzo[h][1,6]naphthyridine-1(2H)-carboxylic acid, 9-[(cyclopropylamino)carbonyl]-5-(4-ethoxyphenyl)-3,4,4a,5,6,10b-hexahydro-, 2-propenyl ester

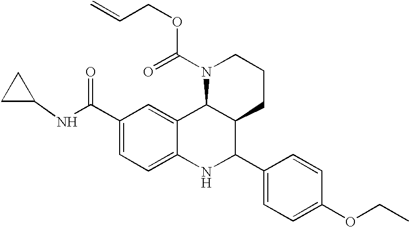 The titled compound (0.91 g; yield, 74%) was obtained by following the general procedure 5

(ESI) (M+H)⁺=476.5.

### Benzo[h][1,6]naphthyridine-1(2H)-carboxylic acid, 5-(4-ethoxyphenyl)-3,4,4a,5,6, 10b-hexahydro-9-[[(2-thienylmethyl)amino]carbonyl]-, 2-propenyl ester

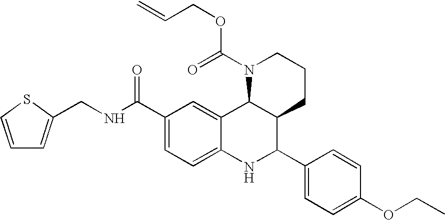 The titled compound (1.10 g; yield, 79%) was obtained by following the general procedure 5

(ESI) (M+H)⁺=532.7.

### Benzo[h][1,6]naphthyridine-1(2H)-carboxylic acid, 5-(4-ethoxyphenyl)-3,4,4a,5,6,10b-hexahydro-9-[[[(5-methyl-2-furanyl)methyl]amino]carbonyl]-, 2-propenyl ester

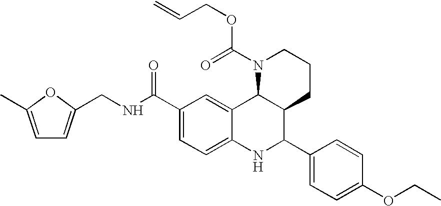 The titled compound (0.80 g; yield, 58%) was obtained by following the general procedure 5

(ESI) (M+H)⁺=530.6.

### Benzo[h][1,6]naphthyridine-1(2H)-carboxylic acid, 9-[(diethylamino)carbonyl]-5-(4-ethoxyphenyl)-3,4,4a,5,6,10b-hexahydro-, 2-propenyl ester

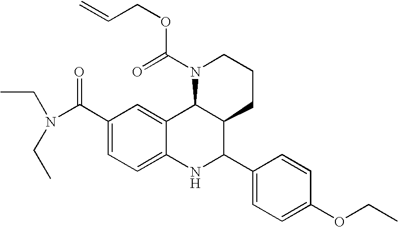 The titled compound (0.83 g; yield, 65%) was obtained by following the general procedure 5

(ESI) (M+H)⁺=492.6.

### Benzo[h][1,6]naphthyridine-1(2H)-carboxylic acid, 5-(4-ethoxyphenyl)-3,4,4a,5,6,10b -hexahydro-9-[[[2-(1-pyrrolidinyl)ethyl]amino]carbonyl]-, 2-propenyl ester

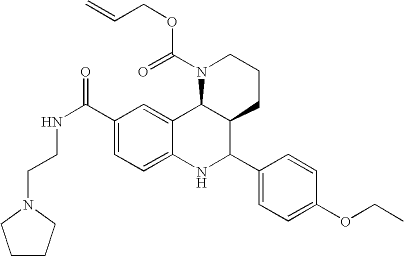 The titled compound (1.03 g; yield, 74%) was obtained by following the general procedure 5

(ESI) (M+H)⁺=533.6.

### Benzo[h][1,6]naphthyridine-1(2H)-carboxylic acid, 3,4,4a,5,6,10b-hexahydro-5-phenyl-9-(1-pyrrolidinylcarbonyl)-, 2-propenyl ester

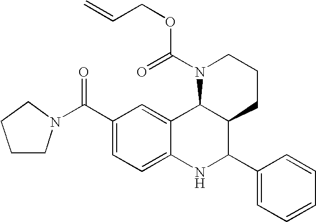 The titled compound (0.62 g, 53%) was obtained by following the general procedure 5

(ESI) (M+H)⁺=446.5.

### Benzo[h][1,6]naphthyridine-1(2H)-carboxylic acid, 3,4,4a,5,6,10b-hexahydro-9-[[(2-methoxyethyl)amino]carbonyl]-5-phenyl-, 2-propenyl ester

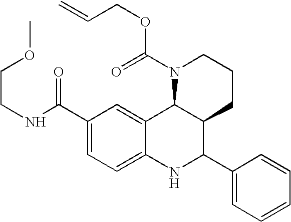 The titled compound (0.62 g; yield, 53%) was obtained by following the general procedure 5

(ESI) (M+H)⁺=450.5.

### Benzo[h][1,6]naphthyridine-1(2H)-carboxylic acid, 9-[(cyclopentylamino)carbonyl]-3,4,4a,5,6,10b-hexahydro-5-phenyl-, 2-propenyl ester

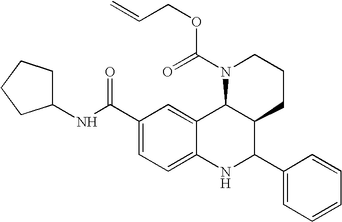 The titled compound (1.014 g; yield, 85%) was obtained by following the general procedure 5

(ESI) (M+H)⁺=460.6.

### Benzo[h][1,6]naphthyridine-1(2H)-carboxylic acid, 9-[(cyclopropylamino)carbonyl]-3,4,4a,5,6,10b-hexahydro-5-phenyl-, 2-propenyl ester

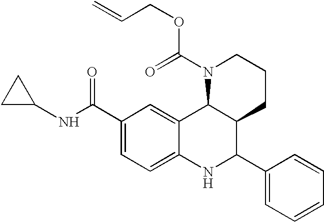 The titled compound (0.91 g; yield, 81%) was obtained by following the general procedure 5

(ESI) (M+H)⁺=432.5.

### Benzo[h][1,6]naphthyridine-1(2H)-carboxylic acid, 3,4,4a,5,6,10b-hexahydro-5-phenyl-9-[[(2-thienylmethyl)amino]carbonyl]-, 2-propenyl ester

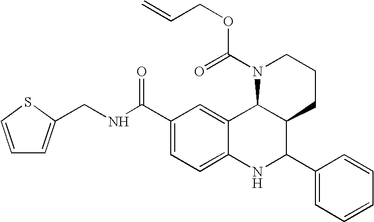 The titled compound (0.606 g; yield, 48%) was obtained by following the general procedure 5

(ESI) (M+H)⁺=488.6.

### Benzo[h][1,6]naphthyridine-1(2H)-carboxylic acid, 3,4,4a,5,6,10b-hexahydro-9-[[[(5-methyl-2-furanyl)methyl]amino]carbonyl]-5-phenyl-, 2-propenyl ester

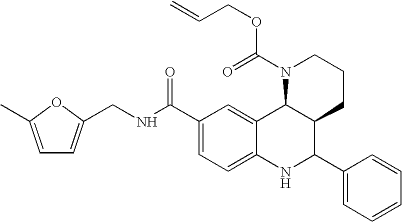 The titled compound (0.768 g; yield, 61%) was obtained by following the general procedure 5

(ESI) (M+H)⁺=486.6.

### Benzo[h][1,6]naphthyridine-1(2H)-carboxylic acid, 9-[(diethylamino)carbonyl]-3,4,4a,5,6,10b -hexahydro-5-phenyl-, 2-propenyl ester

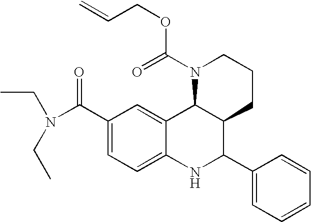 The titled compound (0.717 g; yield, 71%) was obtained by following the general procedure 5

(ESI) (M+H)⁺=448.6.

### Benzo[h][1,6]naphthyridine-1(2H)-carboxylic acid, 3,4,4a,5,6,10b-hexahydro-5-phenyl-9-[[[2-(1-pyrrolidinyl)ethyl]amino]carbonyl]-, 2-propenyl ester

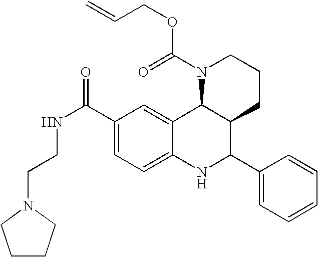 The titled compound (0.95 g; yield, 75%) was obtained by following the general procedure 5

(ESI) (M+H)⁺=489.6.

### Benzo[h][1,6]naphthyridine-1(2H)-carboxylic acid, 3,4,4a,5,6,10b-hexahydro-5-phenyl-9-[[[2-(1-pyrrolidinyl)ethyl]amino]carbonyl]-, 2-propenyl ester

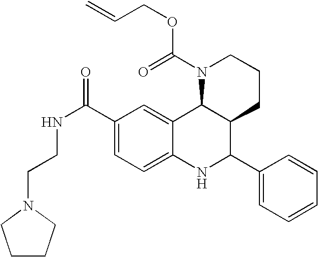 The titled compound (0.95 g; yield, 75%) was obtained by following the general procedure 5

(ESI) (M+H)⁺=489.6.

### Benzo[h][1,6]naphthyridine-1(2H)-carboxylic acid, 6-ethyl-3,4,4a,5,6,10b-hexahydro-5-phenyl-9-(1-pyrrolidinylcarbonyl)-, 2-propenyl ester

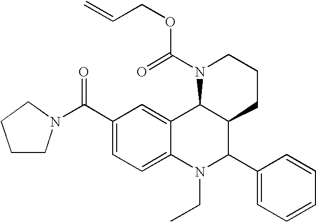 The titled compound (0.62 g; yield, 53%) was obtained by following the general procedure 5

(ESI) (M+H)⁺=446.5.

### Benzo[h][1,6]naphthyridine-1(2H)-carboxylic acid, 6-ethyl-3,4,4a,5,6,10b-hexahydro-9-[[(2-methoxyethyl)amino]carbonyl]-5-phenyl-, 2-propenyl ester

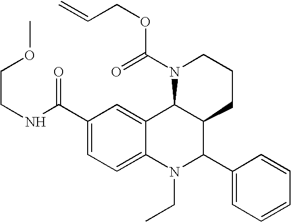 The titled compound (0.94 g; yield, 76%) was obtained by following the general procedure 5

(ESI) (M+H)⁺=478.6.

### Benzo[h][1,6naphthyridine-1(2H)-carboxylic acid, 9-[(cyclopentylamino)carbonyl]-6-ethyl-3,4,4a,5,6,10b-hexahydro-5-phenyl-, 2-propenyl ester

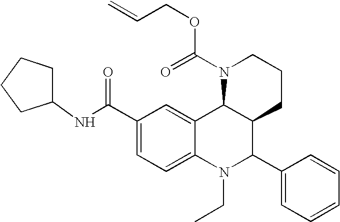 The titled compound (0.975 g; yield, 77%) was obtained by following the general procedure 5

(ESI) (M+H)⁺=488.6.

### Benzo[h][1,6]naphthyridine-1(2H)-carboxylic acid, 9-[(cyclopropylamino)carbonyl]-6-ethyl-3,4,4a,5,6,10b-hexahydro-5-phenyl-, 2-propenyl ester

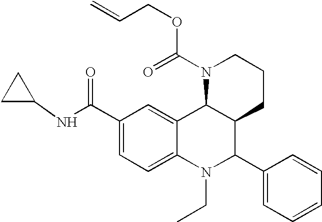 The titled compound (0.524 g; yield, 44%) was obtained by following the general procedure 5

(ESI) (M+H)⁺=432.5.

### Benzo[h][1,6]naphthyridine-1(2H)-carboxylic acid, 6-ethyl-3,4,4a,5,6,10b-hexahydro-5-phenyl-9-[[(2-thienylmethyl)amino]carbonyl]-, 2-propenyl ester

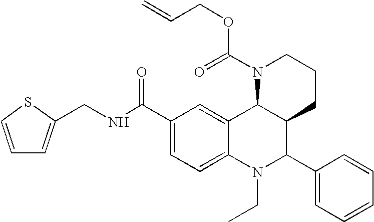 The titled compound (0.761 g; yield, 57%) was obtained by following the general procedure 5

(ESI) (M+H)⁺=516.7.

### Benzo[h][1,6]naphthyridine-1(2H)-carboxylic acid, 6-ethyl-3,4,4a,5,6,10b-hexahydro -9-[[[(5-methyl-2-furanyl)methyl]amino]carbonyl]-5-phenyl-, 2-propenyl ester

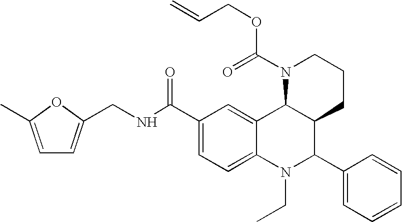 The titled compound (0.740 g; yield, 55%) was obtained by following the general procedure 5

(ESI) (M+H)⁺=514.6.

### benzo[h][1,6]naphthyridine-1(2H)-carboxylic acid, 9-[(diethylamino)carbonyl]-6-ethyl-3,4,4a,5,6,10b-hexahydro-5-phenyl-, 2-propenyl ester

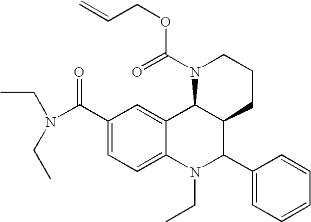 The titled compound (0.840 g; yield, 68%) was obtained by following the general procedure 5

(ESI) (M+H)⁺=476.6.

### Benzo[h][1,6]naphthyridine-1(2H)-carboxylic acid, 6-ethyl-3,4,4a,5,6,10b-hexahydro-5-phenyl-9-[[[2-(1-pyrrolidinyl)ethyl]amino]carbonyl]-, 2-propenyl ester

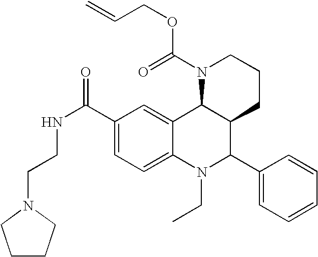 The titled compound (1.062 g; yield, 79%) was obtained by following the general procedure 5

(ESI) (M+H)⁺=517.7.

### Example 12

The titled compounds of Example 12 are made using the titled compounds made in Example 11 as the starting materials.

### Benzo[h][1,6]naphthyridine-9-carboxamide, 5-(4-ethoxyphenyl)-1,2,3,4,4a,5,6,10b-octahydro-N-(2-methoxyethyl)-

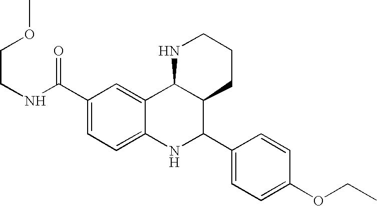 The titled compound (0.655 g; yield, 94%) was obtained by following the general procedure 6

(ESI) (M+H)⁺=410.5.

### Benzo[h][1,6]naphthyridine-9-carboxamide, N-cyclopentyl-5-(4-ethoxyphenyl)-1,2,3,4,4a,5,6,10b -octahydro-

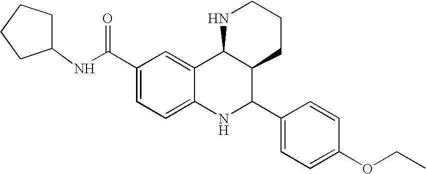 The titled compound (0.625 g; yield, 88%) was obtained by following the general procedure 6

(ESI) (M+H)⁺=420.6.

### Benzo[h][1,6]naphthyridine-9-carboxamide, N-cyclopropyl-5-(4-ethoxyphenyl)-1, 2,3,4,4a,5,6,10b -octahydro-

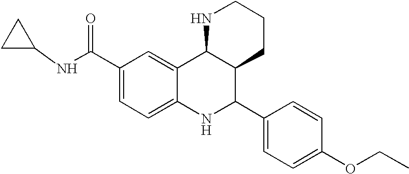 The titled compound (0.609 g; yield, 91%) was obtained by following the general procedure 6

(ESI) (M+H)⁺=392.5.

### Benzo[h][1,6]naphthyridine-9-carboxamide, 5-(4-ethoxyphenyl)-1,2,3,4,4a,5,6,10b-octahydro-N-(2-thienylmethyl)-

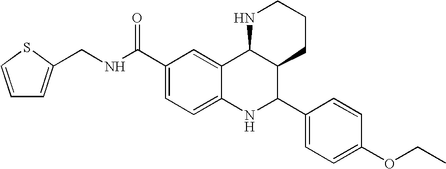 The titled compound (0.708 g; yield, 93%) was obtained by following the general procedure 6

(ESI) (M+H)⁺=448.6.

### Benzo[h][1,6]naphthyridine-9-carboxamide, 5-(4-ethoxyphenyl)-1,2,3,4,4a,5,6,10b-octahydro-N-[(5-methyl-2-furanyl)methyl]-

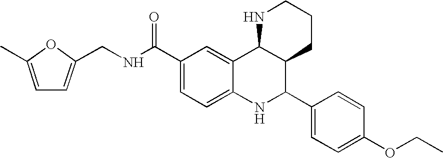 The titled compound (0.735; yield, 97%) was obtained by following the general procedure 6

(ESI) (M+H)⁺=446.6.

### Benzo[h][1,6]naphthyridine-9-carboxamide, 5-(4-ethoxyphenyl)-N,N-diethyl-1,2,3,4,4a,5,6,10b-octahydro-

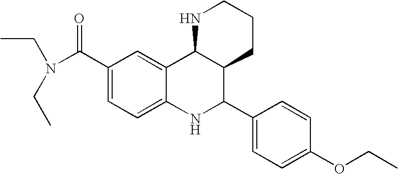 The titled compound (0.603 g; yield, 87%) was obtained by following the general procedure 6

(ESI) (M+H)⁺=408.5.

### Benzo[h][1,6]naphthyridine-9-carboxamide, 5-(4-ethoxyphenyl)-1,2,3,4,4a,5,6,10b-octahydro-N-[2-(1-pyrrolidinyl)ethyl]-

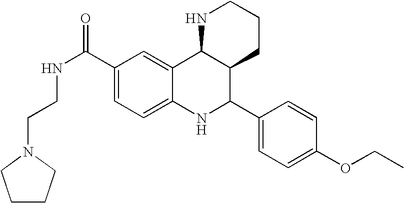 The titled compound (0.755 g; yield, 99%) was obtained by following the general procedure 6

(ESI) (M+H)⁺=449.6.

### Pyrrolidine, 1-[(1,2,3,4,4a,5,6,10b-octahydro-5-phenylbenzo[h][1,6]naphthyridin-9-yl)carbonyl]-

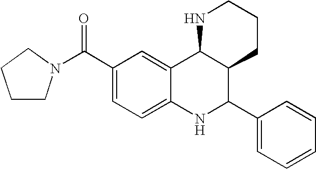 The titled compound (0.609 g; yield, 99%) was obtained by following the general procedure 6

(ESI) (M+H)⁺=362.5.

### Benzo[h][1,6]naphthyridine-9-carboxamide, 1,2,3,4,4a,5,6,10b-octahydro-N-(2-methoxyethyl)-5-phenyl-

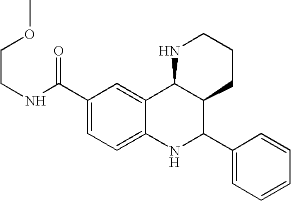 The titled compound (0.578 g; yield, 93%) was obtained by following the general procedure 6

(ESI) (M+H)⁺=366.5.

### Benzo[h][1,6]naphthyridine-9-carboxamide, N-cyclopentyl-1,2,3,4,4a,5,6,10b-octahydro-5-phenyl-

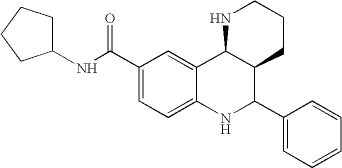 The titled compound (0.556 g; yield, 87%) was obtained by following the general procedure 6

(ESI) (M+H)⁺=376.5.

### Benzo[h][1,6]naphthyridine-9-carboxamide, N-cyclopropyl-1,2,3,4,4a,5,6,10b-octahydro-5-phenyl-

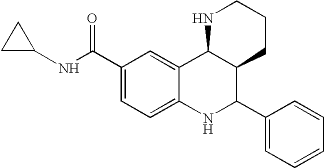 The titled compound (0.503 g; yield, 85%) was obtained by following the general procedure 6

(ESI) (M+H)⁺=348.4.

### Benzo[h][1,6]naphthyridine-9-carboxamide, 1,2,3,4,4a,5,6,10b-octahydro-5-phenyl-N-(2-thienylmethyl)-

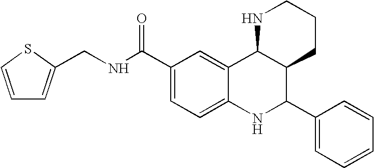 The titled compound (0.659 g; yield, 96%) was obtained by following the general procedure 6

(ESI) (M+H)⁺=404.5.

### Benzo[h][1,6]naphthyridine-9-carboxamide, 1,2,3,4,4a,5,6,10b-octahydro-N-[(5-methyl-2-furanyl)methyl]-5-phenyl-

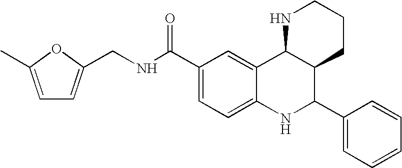 The titled compound (0.643 g; yield, 93%) was obtained by following the general procedure 6

(ESI) (M+H)⁺=402.5.

### Benzo[h][1,6]naphthyridine-9-carboxamide, N,N-diethyl-1,2,3,4,4a,5,6,10b-octahydro-5-phenyl-

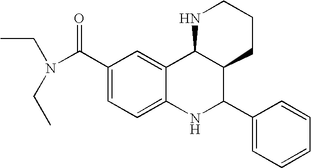 The titled compound (0.600 g; yield, 97%) was obtained by following the general procedure 6

(ESI) (M+H)⁺=364.5.

### Benzo[h][1,6]naphthyridine-9-carboxamide, 1,2,3,4,4a,5,6,10b-octahydro-5-phenyl-N-[2-(1-pyrrolidinol)ethyl]-

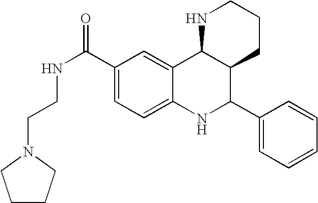 The titled compound (0.544 g; yield, 78%) was obtained by following the general procedure 6

(ESI) (M+H)⁺=405.5.

### Pyrrolidine, 1-[(6-ethyl-1,2,3,4,4a,5,6,10b-octahydro-5-phenylbenzo[h][1,6]naphthyridin-9-yl)carbonyl]-

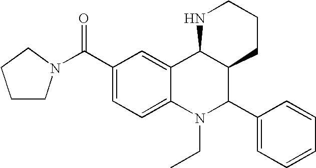 The titled compound (0.590 g; yield, 87%) was obtained by following the general procedure 6

(ESI) (M+H)⁺=390.5.

### Benzo[h][1,6]naphthyridine-9-carboxamide, 6-ethyl-1,2,3,4,4a,5,6,10b-octahydro-N-(2-methoxyethyl)-5-phenyl-

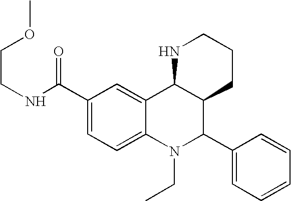 The titled compound (0.634 g; yield, 95%) was obtained by following the general procedure 6

(ESI) (M+H)⁺=394.5.

### Benzo[h][1,6]naphthyridine-9-carboxamide, N-cyclopentyl-6-ethyl-1,2,3,4,4a,5,6,10b -octahydro-5-phenyl-

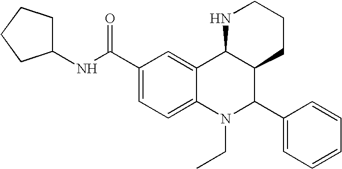 The titled compound (0.637 g; yield, 93%) was obtained by following the general procedure 6

(ESI) (M+H)⁺=404.6.

### N-Cyclopropyl-6-ethyl-5-phenyl-1,2,3,4,4a,5,6,10b-octahydrobenzo[h]-1,6-naphthyridine-9-carboxamide

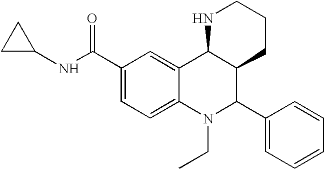 The titled compound (0.556 g; yield, 87%) was obtained by following the general procedure 6

(ESI) (M+H)⁺=376.5.

### 6-Ethyl-5-phenyl-N-(thien-2-ylmethyl)-1,2,3,4,4a,5,6,10b-octahydrobenzo[h]-1,6-naphthyridine-9-carboxamide

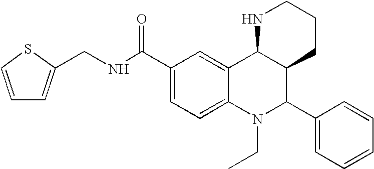 The titled compound (0.668 g; yield, 91%) was obtained by following the general procedure 6

(ESI) (M+H)⁺=432.6.

### 6-Ethyl-N-[(5-methyl-2-furyl)methyl]-5-phenyl-1,2,3,4,4a,5,6,10b-octahydrobenzo[h]-1,6-naphthyridine-9-carboxamide

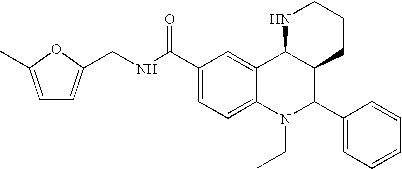 The titled compound (0.723 g; yield, 99%) was obtained by following the general procedure 6

(ESI) (M+H)⁺=430.6.

### N,N,6-Triethyl-5-phenyl]-1,2,3,4,4a,5,6,10b-octahydrobenzo[h]-1,6-naphthyridine-9-carboxamide

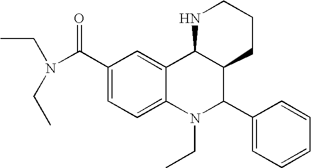 The titled compound (0.580 g; yield, 87%) was obtained by following the general procedure 6

(ESI) (M+H)⁺=392.5.

### 6-Ethyl-5-phenyl-N-(2-pyrrolidin-1-ylethyl)-1,2,3,4,4a,5,6,10b-octahydrobenzo[h]-1,6-naphthyridine-9-carboxamide

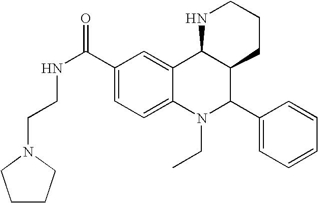 The titled compound (0.618 g; yield, 84%) was obtained by following the general procedure 6

(ESI) (M+H)⁺=433.6.

### Example 13

The titled compounds of Example 12 are reacted with the R⁵COCl listed below in plate format to form the compounds of the present invention using General Procedure 17 below.

General Procedure 17 (Amide formation) 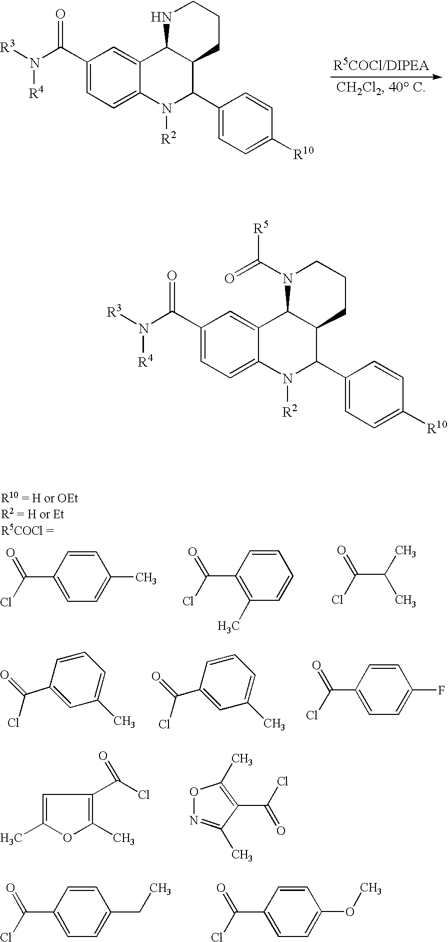
The compounds of Example 13 were prepared by following the general procedure 12.

### Example 14

The titled compounds of Example 12 are reacted with the R⁶SO₂Cl listed below in plate format to form the compounds of the present invention using General Procedure 18 below.

General Procedure 18 (Sulphonyl amide formation) 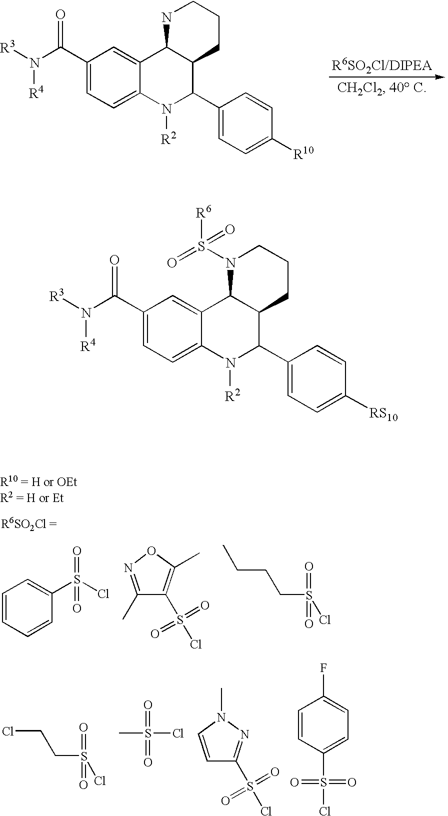
The general procedure 18 is same as the general procedure 13.

### Example 15

The titled compounds of Example 12 are reacted with the R⁷NCX listed below in plate format to form the compounds of the present invention using General Procedure 19 below.

General Procedure 19 (urea or thio urea formation): 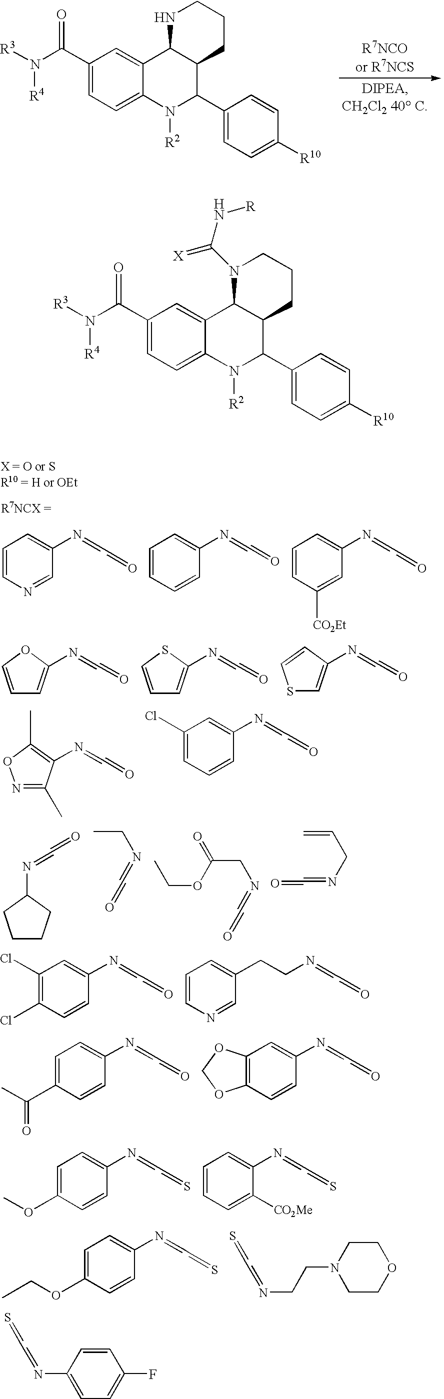
The general procedure 19 is same as the general procedure 14.

### Example 16

The titled compounds of Example 12 are reacted with the R⁸CHO listed below in plate format to form additional compounds of the present invention using General Procedure 20 below.

General Procedure 20 (Reductive amination) 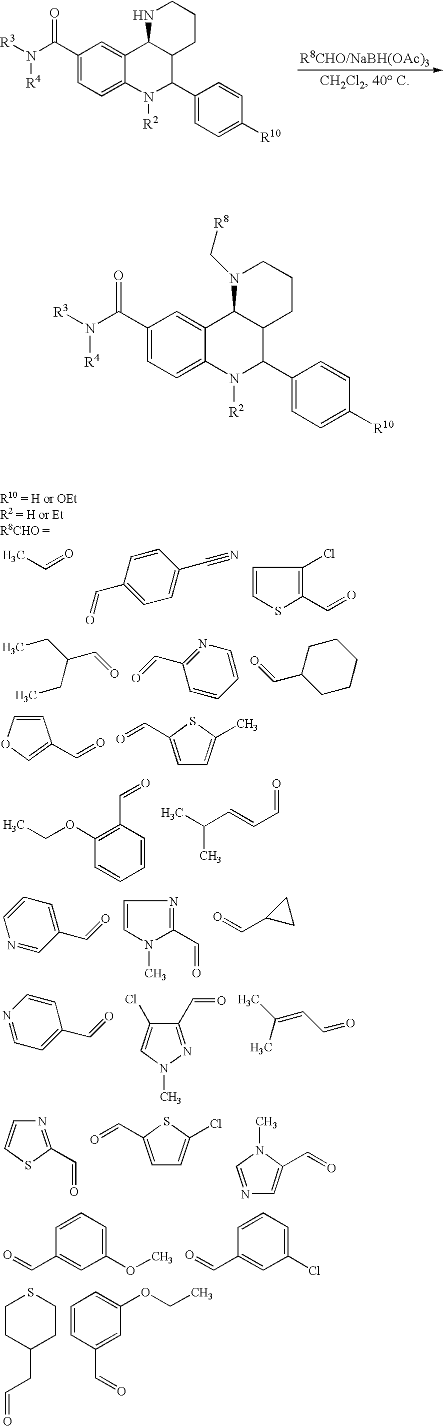 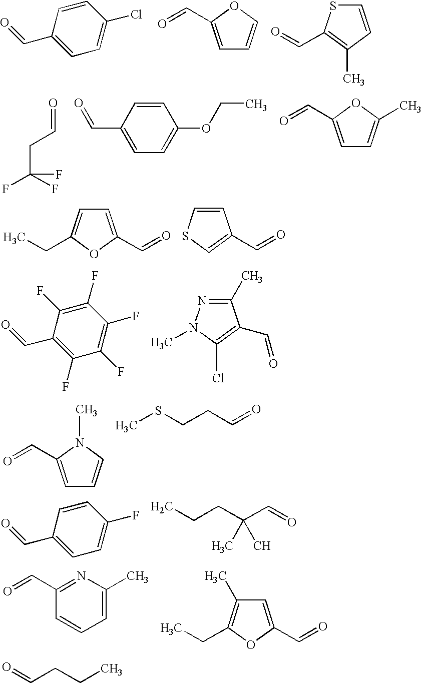
General Procedure 20 is same as the general procedure 15.
In EXAMPLES 13-16, 1040 compounds (13 plates) were prepared. 10 out of every 80 compounds were checked for purity. The purity analysis was performed by analytical LCMS (UV detection). The purity check showed that 80% of selected compounds have purity over 50%. The estimated material in each well is around 10-12 mg.

### Example 17

### 1-[(Allyloxy)carbonyl]-4-(3-thienyl)-2,3,3a,4,5,9b-hexahydro-1H-pyrrolo[3,2-c]quinoline-8-carboxylic acid

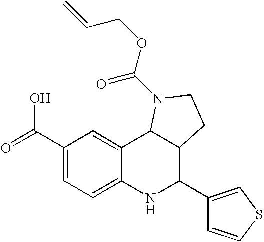 The titled compound (10.7 g; yield, 59%) was obtained by following the general procedure 3.

¹HNMR (400 MHz, CDCl₃): 8.23 (1H,m), 7.75 (1H, m), 7.37 (1H, m), 7.13 (1H,m), 6.62 (1H, m), 5.35 (m, 4H), 4.92 (1H, m), 4.82 (0.4H,m), 4.67 (1.6H, m), 3.82 (2H,m), 2.52 (1H,m), 2.17 (1H,m), 1.53 (1H,m).

(ESI) (M+H)⁺=385.4.

### Allyl 8-[(dimethylamino)carbonyl]-4-(4-ethoxyphenyl)-2,3,3a,4,5,9b-hexahydro-1H-pyrrolo[3,2-c]quinoline-1-carboxylate

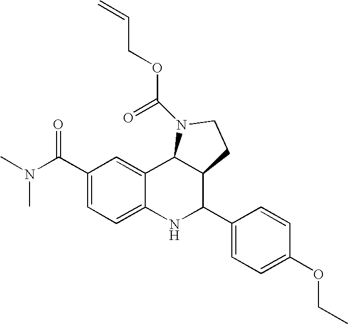 The titled compound (1.31 g; yield, 88%) was obtained by following the general procedure 5

(ESI) (M+H)⁺=450.5.

### Allyl 4-(4-ethoxyphenyl)-8-[(methylamino)carbonyl]-2,3,3a,4,5,9b-hexahydro-1H-pyrrolo[3,2-c]quinoline-1-carboxylate

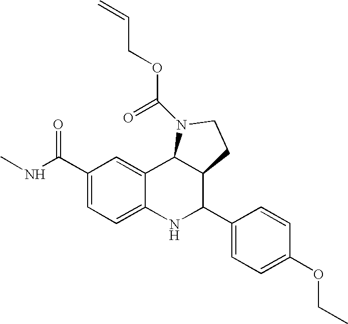 The titled compound (1.48 g; yield, 100%) was obtained by following the general procedure 5

(ESI) (M+H)⁺=436.5.

### Allyl 8-{[(cyclopropylmethyl)amino]carbonyl}-4-(4-ethoxyphenyl)-2,3,3a,4,5,9b-hexahydro-1H -pyrrolo[3,2-c]quinoline-1-carboxylate

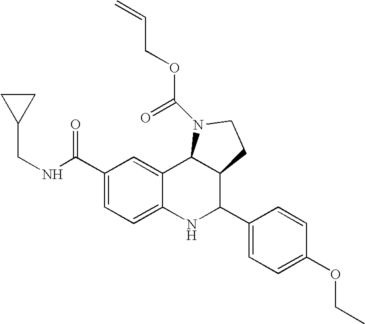 The titled compound (1.24 g; yield, 79%) was obtained by following the general procedure 5

(ESI) (M+H)⁺=476.6.

### Allyl 8-[(cyclobutylamino)carbonyl]-4-(4-ethoxyphenyl)-2,3,3a,4,5,9b-hexahydro-1H -pyrrolo[3,2-c]quinoline-1-carboxylate

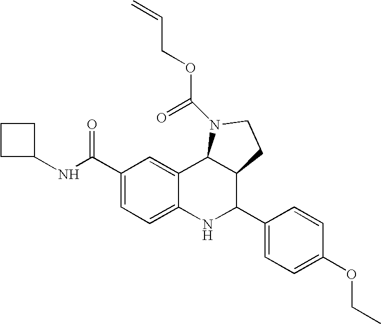 The titled compound (1.5 g; yield, 95%) was obtained by following the general procedure 5

(ESI) (M+H)⁺=476.6.

### Allyl 8-[(cyclopropylamino)carbonyl]-4-(4-ethoxyphenyl)-2,3,3a,4,5,9b-hexahydro-1H -pyrrolo[3,2-c]quinoline-1-carboxylate

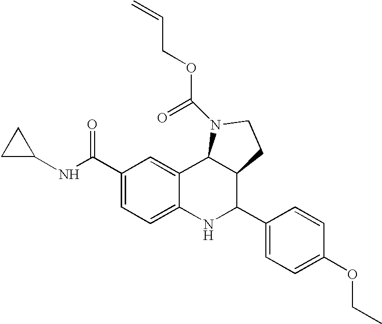 The titled compound (1.563 g; yield, 98%) was obtained by following the general procedure 5

(ESI) (M+H)⁺=462.5.

### Allyl 8-[(allylamino)carbonyl]-4-(4-ethoxyphenyl)-2,3,3a,4,5,9b-hexahydro-1H-pyrrolo[3,2-c]quinoline-1-carboxylate

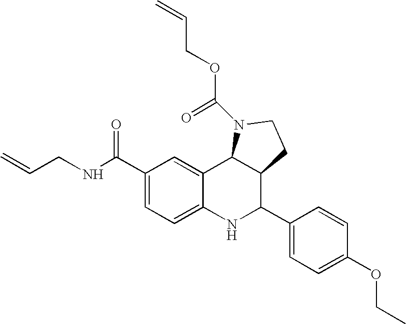 The titled compound (1.563 g; yield, 80%) was obtained by following the general procedure 5

(ESI) (M+H)⁺=462.5.

### Allyl 4-(4-ethoxyphenyl)-8-(piperidin-1-ylcarbonyl)-2,3,3a,4,5,9b-hexahydro-1H-pyrrolo[3,2-c]quinoline-1-carboxylate

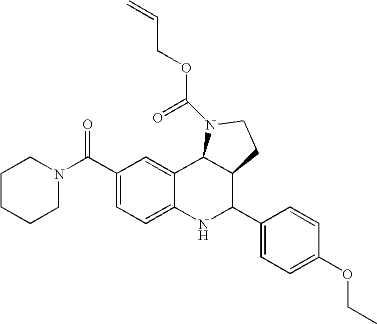 The titled compound (1.568 g; yield, 97%) was obtained by following the general procedure 5

(ESI) (M+H)⁺=490.6.

### Allyl 8-(azetidin-1-ylcarbonyl)-4-(4-ethoxyphenyl)-2,3,3a,4,5,9b-hexahydro-1H-pyrrolo[3,2-c]quinoline-1-carboxylate

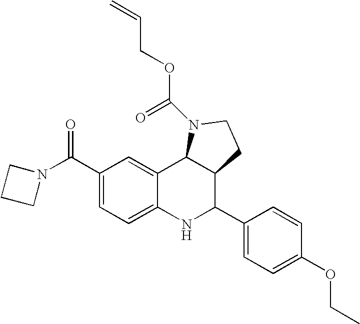 The titled compound (1.116 g; yield, 73%) was obtained by following the general procedure 5

(ESI) (M+H)⁺=462.5.

### Allyl 8-[(dimethylamino)carbonyl]-4-phenyl-2,3,3a,4,5,9b-hexahydro-1H-pyrrolo[3,2-c]quinoline-1-carboxylate

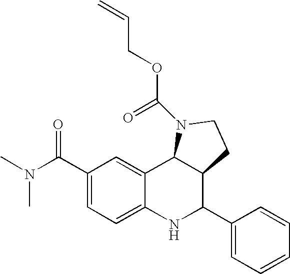 The titled compound (1.283 g; yield, 95%) was obtained by following the general procedure 5

(ESI) (M+H)⁺=406.5.

### Allyl (3aS,9bS)-8-[(methylamino)carbonyl]-4-phenyl-2,3,3a,4,5,9b-hexahydro-1H-pyrrolo[3,2-c]quinoline-1-carboxylate

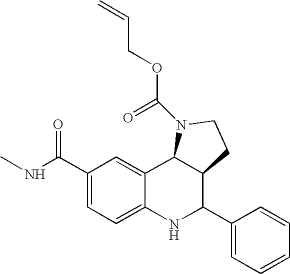 The titled compound (1.283 g; yield, 96%) was obtained by following the general procedure 5

(ESI) (M+H)⁺=392.5.

### Allyl-{[(cyclopropylmethyl)amino]carbonyl}-4-phenyl-2,3,3a,4,5,9b-hexahydro-1H-pyrrolo[3,2-c]quinoline-1-carboxylate

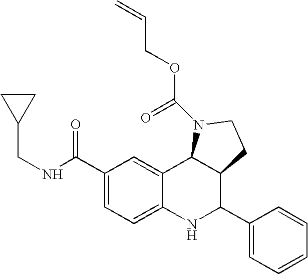 The titled compound (1.295 g; yield, 91%) was obtained by following the general procedure 5

(ESI) (M+H)⁺=432.5.

### Allyl 8-[(cyclobutylamino)carbonyl]-4-phenyl-2,3,3a,4,5,9b-hexahydro-1H-pyrrolo[3,2-c]quinoline-1-carboxylate

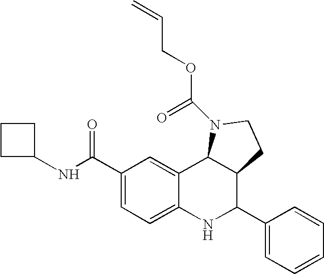 The titled compound (1.12 g; yield, 78%) was obtained by following the general procedure 5

(ESI) (M+H)⁺=432.5.

### Allyl 8-[(cyclopropylamino)carbonyl]-4-phenyl-2,3,3a,4,5,9b-hexahydro-1H-pyrrolo[3,2-c]quinoline-1-carboxylate

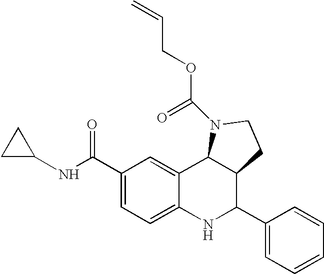 The titled compound (1.07 g; yield, 78%) was obtained by following the general procedure 5

(ESI) (M+H)⁺=418.5.

### Allyl 8-[(allylamino)carbonyl]-4-phenyl-2,3,3a,4,5,9b-hexahydro-1H-pyrrolo[3,2-c]quinoline-1-carboxylate

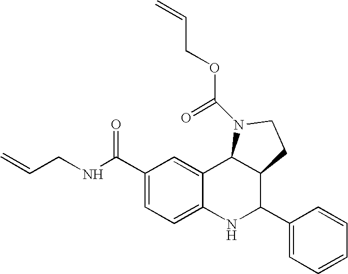 The titled compound (1.134 g; yield, 82%) was obtained by following the general procedure 5

(ESI) (M+H)⁺=418.5.

### Allyl 4-phenyl-8-(piperidin-1-ylcarbonyl)-2,3,3a,4,5,9b-hexahydro-1H-pyrrolo[3,2-c]quinoline-1-carboxylate

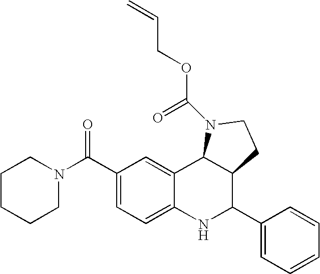 The titled compound (1.463 g; yield, 99%) was obtained by following the general procedure 5

(ESI) (M+H)⁺=446.5.

### Allyl 8-(azetidin-1-ylcarbonyl)-4-phenyl-2,3,3a,4,5,9b-hexahydro-1H-pyrrolo[3,2-c]quinoline-1-carboxylate

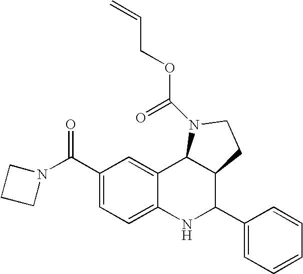 The titled compound (1.40 g; yield, 100%) was obtained by following the general procedure 5

(ESI) (M+H)⁺=418.5.

### Allyl 8-[(dimethylamino)carbonyl]-4-(2-furyl)-2,3,3a,4,5,9b-hexahydro-1H-pyrrolo[3,2-c]quinoline-1-carboxylate

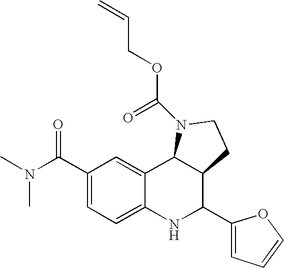 The titled compound (1.30 g; yield, 99%) was obtained by following the general procedure 5

(ESI) (M+H)⁺=396.5.

### Allyl 4-(2-furyl)-8-[(methylamino)carbonyl]-2,3,3a,4,5,9b-hexahydro-1H-pyrrolo[3,2-c]quinoline-1-carboxylate

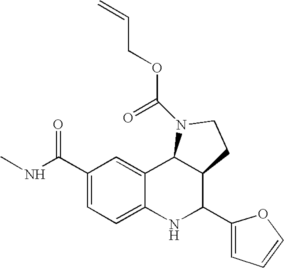 The titled compound (1.30 g; yield, 100%) was obtained by following the general procedure 5

(ESI) (M+H)+382.4

### Allyl 8-{[(cyclopropylmethyl)amino]carbonyl}-4-(2-furyl)-2,3,3a,4,5,9b-hexahydro-1H-pyrrolo[3,2-c]quinoline-1-carboxylate

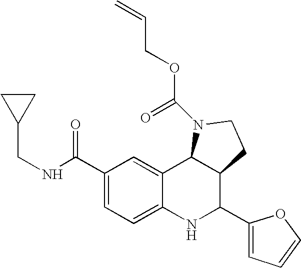 The titled compound (1.20 g; yield, 86%) was obtained by following the general procedure 5

(ESI) (M+H)⁺=422.5.

### Allyl 8-[(cyclobutylamino)carbonyl]-4-(2-furyl)-2,3,3a,4,5,9b-hexahydro-1H-pyrrolo[3,2-c]quinoline-1-carboxylate

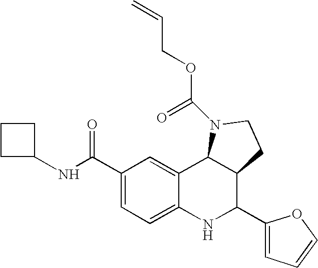 The titled compound (1.13 g; yield, 81%) was obtained by following the general procedure 5

(ESI) (M+H)⁺=422.5.

### Allyl 8-[(cyclopropylamino)carbonyl]-4-(2-furyl)-2,3,3a,4,5,9b-hexahydro-1H-pyrrolo[3,2-c]quinoline-1-carboxylate

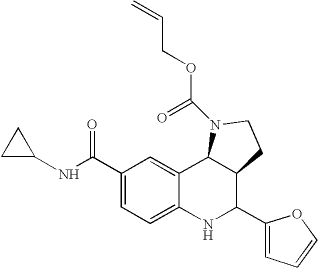 The titled compound (1.27 g; yield, 95%) was obtained by following the general procedure 5

(ESI) (M+H)⁺=408.5.

### Allyl 8-[(allylamino)carbonyl]-4-(2-furyl)-2,3,3a,4,5,9b-hexahydro-1H-pyrrolo[3,2-c]quinoline-1-carboxylate

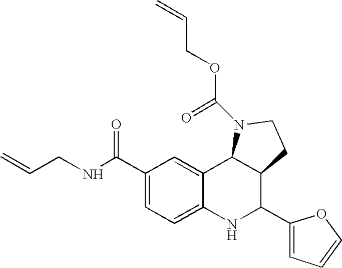 The titled compound (1.25 g; yield, 93%) was obtained by following the general procedure 5

(ESI) (M+H)⁺=408.5.

### Allyl 4-(2-furyl-8-(piperidin-1-ylcarbonyl)-2,3,3a,4,5,9b-hexahydro-1H-pyrrolo[3,2-c]quinoline-1-carboxylate

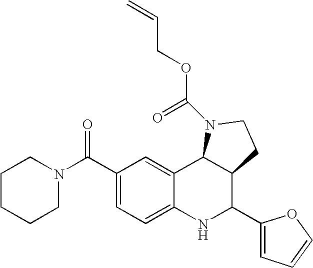 The titled compound (1.25 g; yield, 87%) was obtained by following the general procedure 5

(ESI) (M+H)⁺=436.5.

### Allyl 8-(azetidin-1-ylcarbonyl)-4-(2-furyl)-2,3,3a,4,5,9b-hexahydro-1H-pyrrolo[3,2-c]quinoline-1-carboxylate

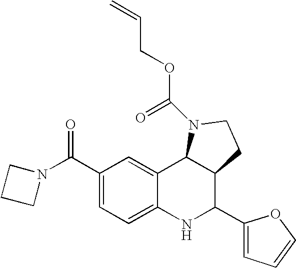 The titled compound (1.214 g; yield, 90%) was obtained by following the general procedure 5

(ESI) (M+H)⁺=408.5.

### Allyl 8-[(dimethylamino)carbonyl]-4-thien-3-yl-2,3,3a,4,5,9b-hexahydro-1H-pyrrolo[3,2-c]quinoline-1-carboxylate

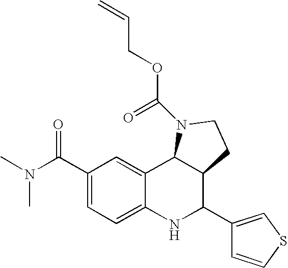 The titled compound (1.285 g; yield, 94%) was obtained by following the general procedure 5

(ESI) (M+H)⁺=412.5.

### Allyl 8-[(methylamino)carbonyl]-4-thien-3-yl-2,3,3a,4,5,9b-hexahydro-1H-pyrrolo[3,2-c]quinoline-1-carboxylate

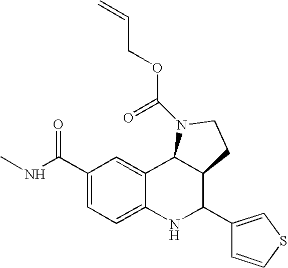 The titled compound (0.966 g; yield, 74%) was obtained by following the general procedure 5

(ESI) (M+H)⁺=398.5.

### Allyl 8-{[(cyclopropylmethyl)amino]carbonyl}-4-thien-3-yl-2,3,3a,4,5,9b-hexahydro-1H-pyrrolo[3,2-c]quinoline-1-carboxylate

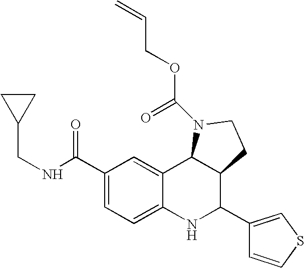 The titled compound (1.08 g; yield, 75%) was obtained by following the general procedure 5

(ESI) (M+H)⁺=438.5.

### Allyl 8-[(cyclobutylamino)carbonyl]-4-thien-3-yl-2,3,3a,4,5,9b-hexahydro-1H-pyrrolo[3,2-c]quinoline-1-carboxylate

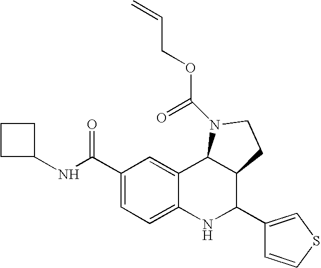 The titled compound (1.048 g; yield, 73%) was obtained by following the general procedure 5

(ESI) (M+H)⁺=438.5.

### Allyl 8-[(cyclopropylamino)carbonyl]-4-thien-3-yl-2,3,3a,4,5,9b-hexahydro-1H-pyrrolo[3,2-c]quinoline-1-carboxylate

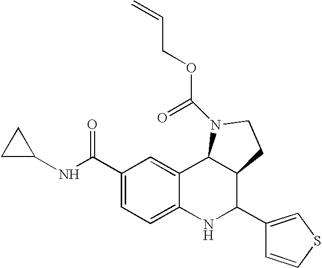 The titled compound (1.20 g; yield, 86%) was obtained by following the general procedure 5

(ESI) (M+H)⁺=438.5.

### Allyl 8-[(alkylamino)carbonyl]-4-thien-3-yl-2,3,3a,4,5,9b-hexahydro-1H-pyrrolo[3,2-c]quinoline-1-carboxylate

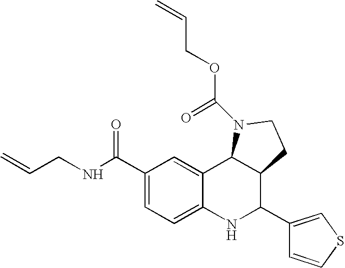 The titled compound (1.421 g; yield, 100%) was obtained by following the general procedure 5

(ESI) (M+H)⁺=424.5.

### Allyl 8-(piperidin-1-ylcarbonyl)-4-thien-3-yl-2,3,3a,4,5,9b-hexahydro-1H-pyrrolo[3,2-c]quinoline-1-carboxylate

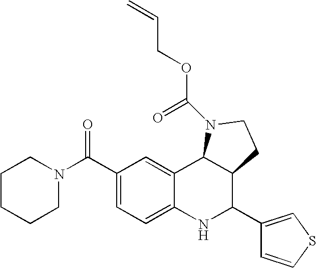 The titled compound (1.49 g; yield, 100%) was obtained by following the general procedure 5

(ESI) (M+H)⁺=452.6.

### Allyl 8-(azetidin-1-ylcarbonyl)-4-thien-3-yl-2,3,3a,4,5,9b-hexahydro-1H-pyrrolo[3,2-c]quinoline-1-carboxylate

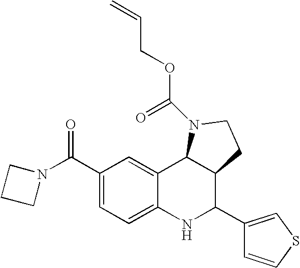 The titled compound (1.157 g; yield, 83%) was obtained by following the general procedure 5

(ESI) (M+H)+424.5.

### Example 18

The titled compounds of Example 18 are made using the titled compounds made in Example 17 as the starting materials.

### 4-(4-Ethoxyphenyl)-N,N-dimethyl-2,3,3a,4,5,9b-hexahydro-1H-pyrrolo[3,2-c]quinoline-8-carboxamide

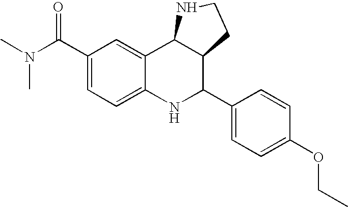 The titled compound (0.848 g; yield, 95%) was obtained by following the general procedure 6

(ESI) (M+H)⁺=365.5.

### 4-(4-Ethoxyphenyl)-N-methyl-2,3,3a,4,5,9b-hexahydro-1H-pyrrolo[3,2-c]quinoline-8-carboxamide

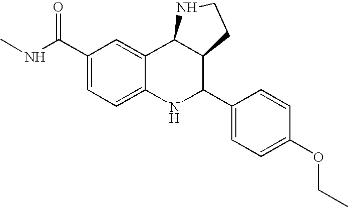 The titled compound (0.751 g; yield, 88%) was obtained by following the general procedure 6

(ESI) (M+H)⁺=352.4.

### N-(Cyclopropylmethyl)-4-(4-ethoxyphenyl)-2,3,3a,4,5,9b-hexahydro-1H-pyrrolo[3,2-c]quinoline-8-carboxamide

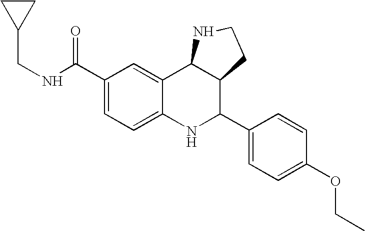 The titled compound (0.893 g; yield, 94%) was obtained by following the general procedure 6

(ESI) (M+H)⁺=392.5.

### N-Cyclobutyl-4-(4-ethoxyphenyl)-2,3,3a,4,5,9b-hexahydro-1H-pyrrolo[3,2-c]quinoline-8-carboxamide

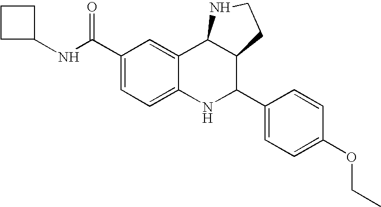 The titled compound (0.809 g; yield, 85%) was obtained by following the general procedure 6

(ESI) (M+H)⁺=391.5.

### N-Cyclopropyl-4-(4-ethoxyphenyl)-2,3,3a,4,5,9b-hexahydro-1H-pyrrolo[3,2-c]quinoline-8-carboxamide

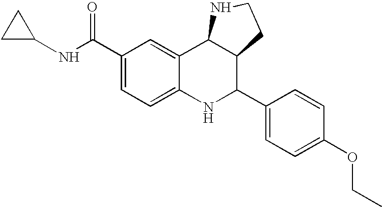 The titled compound (0.824 g; yield, 90%) was obtained by following the general procedure 6

(ESI) (M+H)⁺=378.5.

### N-Allyl-4-(4-ethoxyphenyl)-2,3,3a,4,5,9b-hexahydro-1H-pyrrolo[3,2-c]quinoline-8-carboxamide

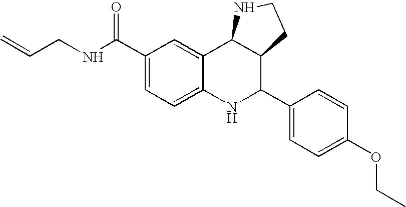 The titled compound (0.801 g; yield, 87%) was obtained by following the general procedure 6

(ESI) (M+H)⁺=378.5.

### 4-(4-Ethoxyphenyl)-8-(piperidin-1-ylcarbonyl)-2,3,3a,4,5,9b-hexahydro-1H-pyrrolo[3,2-c]quinoline

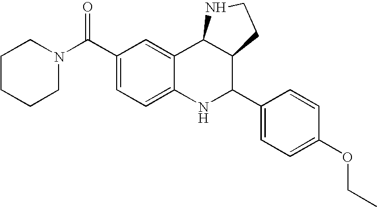 The titled compound (0.962 g; yield, 96%) was obtained by following the general procedure 6

(ESI) (M+H)⁺=406.5.

### 8-(Azetidin-1-ylcarbonyl)-4-(4-ethoxyphenyl)-2,3,3a,4,5,9b-hexahydro-1H-pyrrolo[3,2-c]quinoline

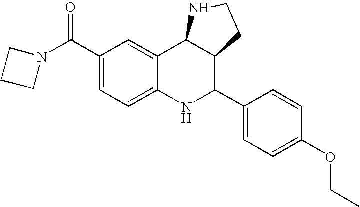 The titled compound (0.872 g; yield, 95%) was obtained by following the general procedure 6

(ESI) (M+H)⁺=378.5.

### N,N-Dimethyl-4-phenyl-2,3,3a,4,5,9b-hexahydro-1H-pyrrolo[3,2-c]quinoline-8-carboxamide

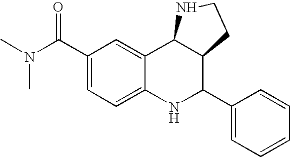 The titled compound (0.722 g; yield, 92%) was obtained by following the general procedure 6

(ESI) (M+H)⁺=322.4.

### N-Methyl-4-phenyl-2,3,3a,4,5,9b-hexahydro-1H-pyrrolo[3,2-c]quinoline-8-carboxamide

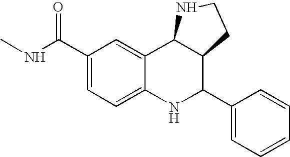 The titled compound (0.697 g; yield, 93%) was obtained by following the general procedure 6

(ESI) (M+H)+308.4.

### N-(Cyclopropylmethyl)-4-phenyl-2,3,3a,4,5,9b-hexahydro-1H-pyrrolo[3,2-c]quinoline-8-carboxamide

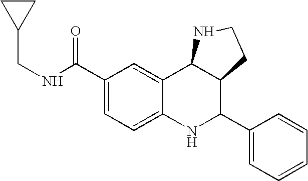 The titled compound (0.807 g; yield, 95%) was obtained by following the general procedure 6

(ESI) (M+H)⁺=348.4.

### N-Cyclobutyl-4-phenyl-2,3,3a,4,5,9b-hexahydro-1H-pyrrolo[3,2-c]quinoline-8-carboxamide

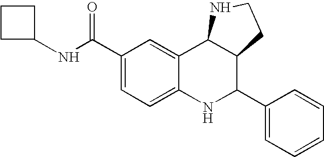 The titled compound (0.740 g; yield, 87%) was obtained by following the general procedure 6

(ESI) (M+H)⁺=348.4.

### N-Cyclopropyl-4-phenyl-2,3,3a,4,5,9b-hexahydro-1H-pyrrolo[3,2-c]quinoline-8-carboxamide

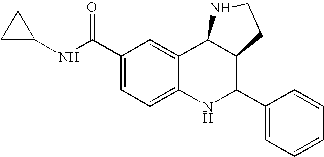 The titled compound (0.692 g; yield, 85%) was obtained by following the general procedure 6

(ESI) (M+H)⁺=334.4.

### N-Allyl-4-phenyl-2,3,3a,4,5,9b-hexahydro-1H-pyrrolo[3,2-c]quinoline-8-carboxamide

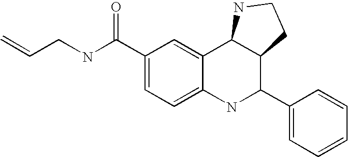 The titled compound (0.779 g; yield, 96%) was obtained by following the general procedure 6

(ESI) (M+H)⁺=334.4.

### 4-Phenyl-8-(piperidin-1-ylcarbonyl)-2,3,3a,4,5,9b-hexahydro-1H-pyrrolo[3,2-c]quinoline

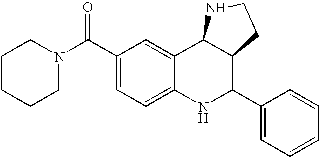 The titled compound (0.848 g; yield, 96%) was obtained by following the general procedure 6.

(ESI) (M+H)⁺=362.5.

### 8-(Azetidin-1-ylcarbonyl)-4-phenol-2,3,3a,4,5,9b-hexahydro-1H-pyrrolo[3,2-c]quinoline

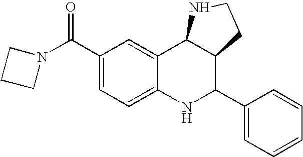 The titled compound (0.703 g; yield, 87%) was obtained by following the general procedure 6

(ESI) (M+H)⁺=334.4.

### 4-(2-Furyl)-N,N-dimethyl-2,3,3a,4,5,9b-hexahydro-1H-pyrrolo[3,2-c]quinoline-8-carboxamide

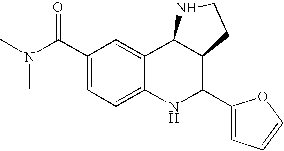 The titled compound (0.678 g; yield, 89%) was obtained by following the general procedure 6

(ESI) (M+H)⁺=312.4.

### 4-(2-Furyl)-N-methyl-2,3,3a,4,5,9b-hexahydro-1H-pyrrolo[3,2-c]quinoline-8-carboxamide

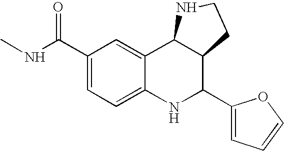 The titled compound (0.713 g; yield, 99%) was obtained by following the general procedure 6

(ESI) (M+H)⁺=298.4

### N-(Cyclopropylmethyl)-4-(2-furyl)-2,3,3a,4,5,9b-hexahydro-1H-pyrrolo[3,2-c]quinoline-8-carboxamide

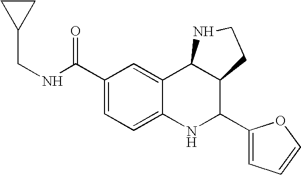 The titled compound (0.647; yield, 79%) was obtained by following the general procedure 6

(ESI) (M+H)⁺=338.4.

### N-Cyclobutyl-4-(2-furyl)-2,3,3a,4,5,9b-hexahydro-1H-pyrrolo[3,2-c]quinoline-8-carboxamide

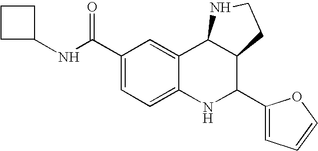 The titled compound (0.792 g; yield, 96%) was obtained by following the general procedure 6

(ESI) (M+H)⁺=338.4.

### N-Cyclopropyl-4-(2-furyl)-2,3,3a,4,5,9b-hexahydro-1H-pyrrolo[3,2-c]quinoline-8-carboxamide

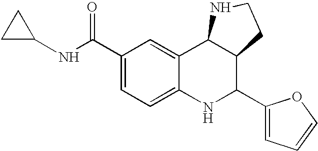 The titled compound (0.698 g; yield, 89%) was obtained by following the general procedure 6

(ESI) (M+H)⁺=324.4.

### N-Allyl-4-(2-furyl)-2,3,3a,4,5,9b-hexahydro-1H-pyrrolo[3,2-c]quinoline-8-carboxamide

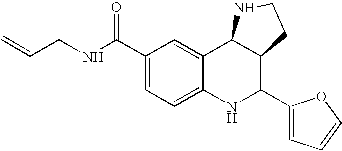 The titled compound (0.729 g; yield, 92%) was obtained by following the general procedure 6

(ESI) (M+H)⁺=324.4.

### 4-(2-Furyl-8-piperidin-1-ylcarbonyl)-2,3,3a,4,5,9b-hexahydro-1H-pyrrolo[3,2-c]quinoline

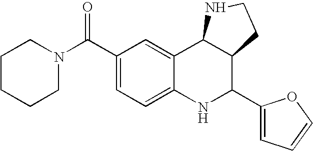 The titled compound (0.739 g; yield, 86%) was obtained by following the general procedure 6

(ESI) (M+H)⁺=352.4.

### 8-(Azetidin-1-ylcarbonyl)-4-(2-furyl)-2,3,3a,4,5,9b-hexahydro-1H-pyrrolo[3,2-c]quinoline

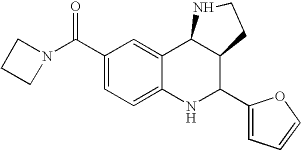 The titled compound (0.777 g; yield, 99%) was obtained by following the general procedure 6

(ESI) (M+H)⁺=324.4.

### N,N-Dimethyl-4-thien-3-yl-2,3,3a,4,5,9b-hexahydro-1H-pyrrolo[3,2-c]quinoline-8-carboxamide

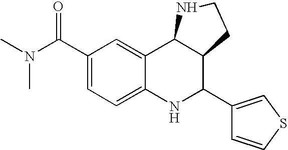 The titled compound (0.713 g; yield, 89%) was obtained by following the general procedure 6

(ESI) (M+H)⁺=328.4.

### N-Methyl-4-thien-3-yl-2,3,3a,4,5,9b-hexahydro-1H-pyrrolo[3,2-c]quinoline-8-carboxamide

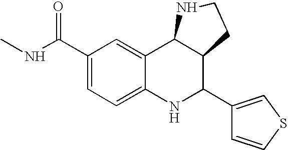 The titled compound (0.659 g; yield, 84%) was obtained by following the general procedure 6

(ESI) (M+H)⁺=314.4.

### N-(Cyclopropylmethyl)-4-thien-3-yl-2,3,3a,4,5,9b-hexahydro-1H-pyrrolo[3,2-c]quinoline-8-carboxamide

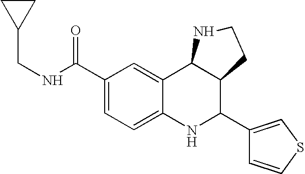 The titled compound (0.765 g; yield, 88%) was obtained by following the general procedure 6

(ESI) (M+H)⁺=354.5.

### N-Cyclobutyl-4-thien-3-yl-2,3,3a,4,5,9b-hexahydro-1H-pyrrolo[3,2-c]quinoline-8-carboxamide

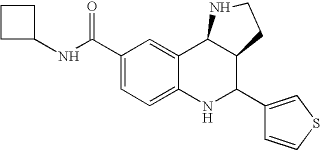 The titled compound (0.851 g; yield, 99%) was obtained by following the general procedure 6

(ESI) (M+H)⁺=354.5.

### N-Cyclopropyl-4-thien-3-yl-2,3,3a,4,5,9b-hexahydro-1H-pyrrolo[3,2-c]quinoline-8-carboxamide

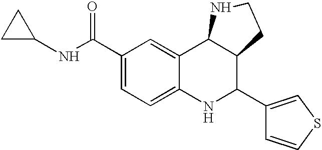 The titled compound (0.780 g; yield, 93%) was obtained by following the general procedure 6

(ESI) (M+H)⁺=340.4.

### N-Allyl-4-thien-3-yl-2,3,3a,4,5,9b-hexahydro-1H-pyrrolo[3,2-c]quinoline-8-carboxamide

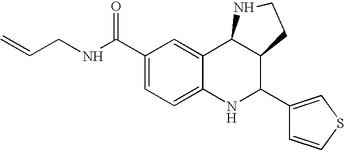 The titled compound (0.714 g; yield, 86%) was obtained by following the general procedure 6

(ESI) (M+H)⁺=340.4.

### 8-(Piperidin-1-ylcarbonyl)-4-thien-3-yl-2,3,3a,4,5,9b-hexahydro-1H-pyrrolo[3,2-c]quinoline

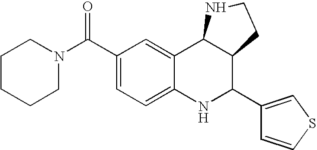 The titled compound (0.856; yield, 96%) was obtained by following the general procedure 6

(ESI) (M+H)⁺=368.5.

### 8-(Azetidin-1-ylcarbonyl)-4-thien-3-yl-2,3,3a,4,5,9b-hexahydro-1H-pyrrolo[3,2-c]quinoline

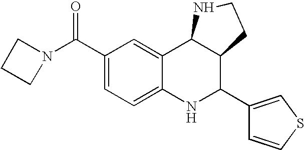 The titled compound (0.740 g; yield, 90%) was obtained by following the general procedure 6

(ESI) (M+H)⁺=340.5.

### N-[2-(Dimethylamino)ethyl]-4-phenyl-2,3,3a,4,5,9b-hexahydro-1H-pyrrolo[3,2-c]quinoline-8-carboxamide

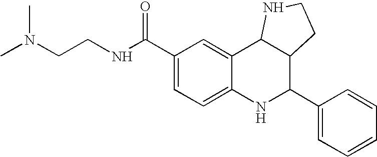 The titled compound (317 mg, yield, 97%) was prepared by following the general procedure 6

(ESI) (M+H)⁺=365.484.

### Example 19

The titled compounds of Example 18 are reacted with the R⁵COCl listed below in plate format to form the compounds of the present invention using General Procedure 21 below.

General Procedure 21 (amide formation) 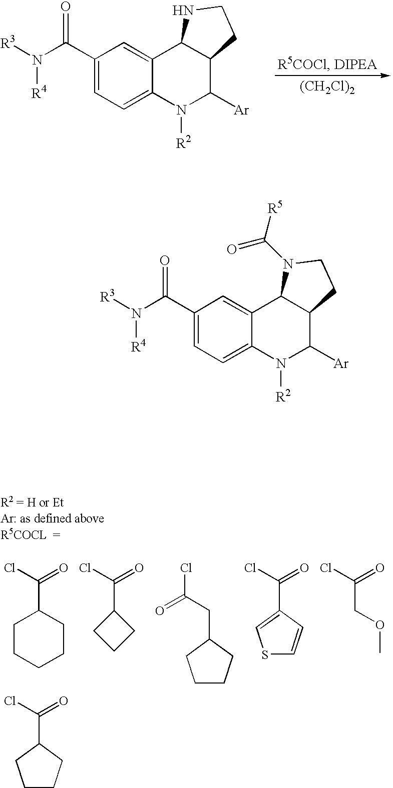
The procedure 21 is same as the general procedure 12.

### Example 20

The titled compounds of Example 18 are reacted with the R⁷NCX listed below in plate format to form the compounds of the present invention using General Procedure 22 below.

General Procedure 22 (urea or thio urea formation):

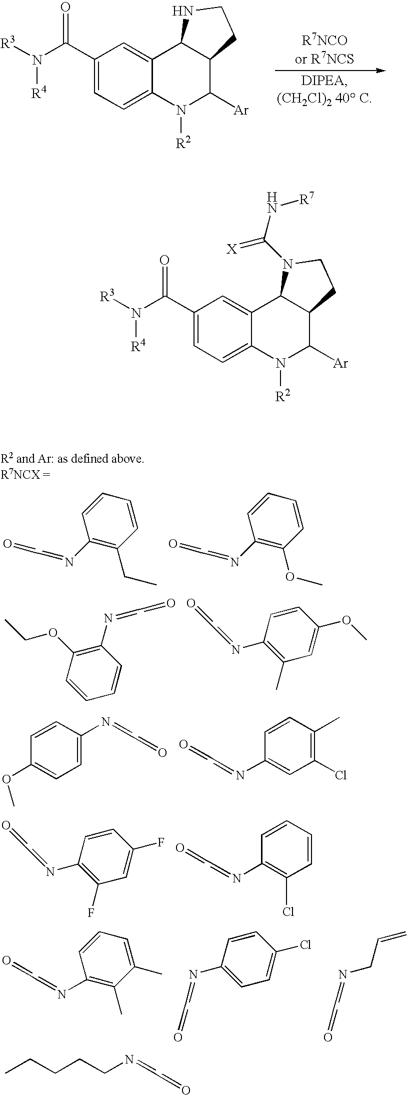

General Procedure 22 is same as the general procedure 14.

### Example 21

The titled compounds of Example 18 are reacted with the R⁵CHO listed below in plate format to form the compounds of the present invention using General Procedure 23 below.

General Procedure 23 (Reductive amination) 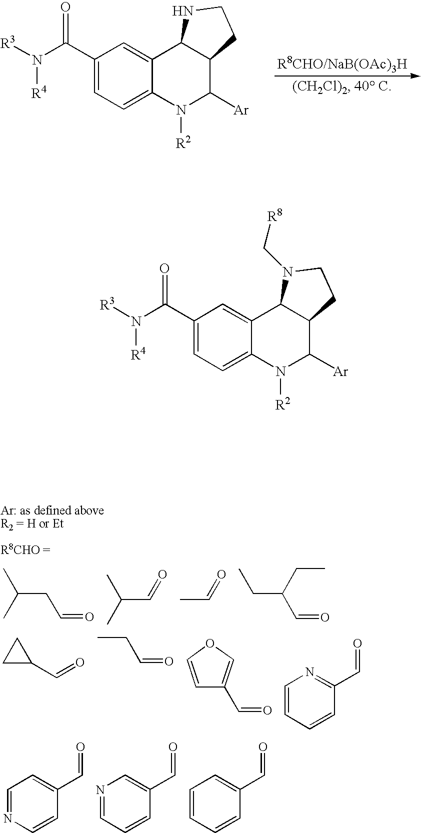
General Procedure 23 is same as the general procedure 15.
In EXAMPLES 19-21, 960 (total 12 plates) compounds were prepared. 90% of the prepared compounds have purity greater than 50%. These compounds obtained directly from the plate chemistry were purified by prep-LCMS. The LC/MS purified 10 compounds were >85% pure and >25 mg was recovered.

### Example 22

### 1-Benzoyl-4-phenyl-8-(pyrrolidin-1-ylcarbonyl)-2,3,3a,4,5,9b-hexahydro-1H-pyrrolo[3,2-c]quinoline

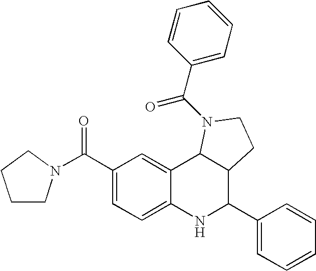 The titled compound (85 mg; yield, 73%) was obtained by following the general procedure 9.

¹H NMR (CDCl₃, 400 MHz): 7.50-7.20 (13H, m), 6.64 (0.44H, d, J=8.4 Hz), 6.62 (0.56H, d, J=8.4 Hz), 4.82 (0.44H, d, J=2.5 Hz), 4.37 (0.56H, d, J=3.9 Hz), 3.57 (6H, m), 2.65 (1H, m), 2.10 (2H, m), 1.87 (4H, m).

(ESI) (M+H)⁺=452.6.

### 1-Benzoyl-N-[2-(diethylamino)ethyl]-4-phenyl-2,3,3a,4,5,9b-hexahydro-1H-pyrrolo[3,2-c]quinoline-8-carboxamide

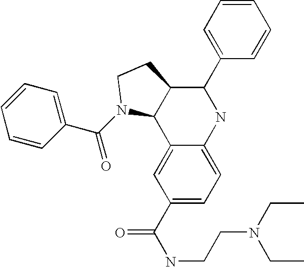 The titled compound (45.2 mg, yield, 67%) was prepared by following the general procedure 9

(ESI) (M+H)⁺=497.651.

### N,N-Diethyl-4-phenyl-1-(phenylsulfonyl)-2,3,3a,4,5,9b-hexahydro-1H-pyrrolo[3,2-c]quinoline-8-carboxamide

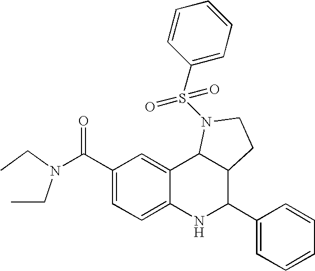 The titled compound (55 mg, yield: 48%) was prepared by following the general procedure 10.

¹H NMR (400 MHz, CDCl₃): ppm 7.78 (1H, d, J=1.0 Hz), 7.68 (1H, dd, J=8.2, 1.0 Hz), 7.56 (1H, m), 7.42 (2H, dd, J=7.8, 7.4 Hz), 7.28 (4H, m), 7.08 (2H, dd, J=7.6, 1.6 Hz), 6.58 (1H, d, J=8.2 Hz), 4.60 (1H, d, J=6.4), 4.21 (1H, d, J=2.7 Hz), 3.42 (7H,m), 1.85 (2H,m), 1.26 (6H, t, J=7.0 Hz). (The ratio of two isomers: 18:1)

MS (ESI) (M+H)⁺=490.63.

### 1-Benzyl-N-[2-(diethylamino)ethyl]-4-phenyl-2,3,3a,4,5,9b-hexahydro-1H-pyrrolo[3,2-c]quinoline-8-carboxamide

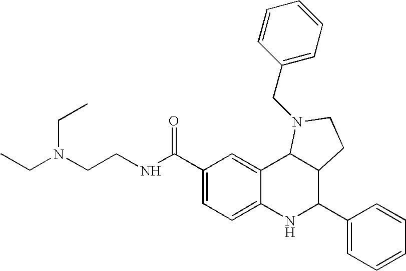 The titled compound (120 mg, 99% yield) was prepared by following the general procedure 8.

¹H-NMR (400 MHz, CD3C₁): 8.05 (m, 1H), 7.78 (m, 1H), 7.60-7.30 (m, 11H), 6.95 (d, J=8.8 Hz, 0.3H), 6.84 (d, J=8.8 Hz, 0.7H), 5.18 (d, J=9.5 Hz, 0.3H), 5.02 (d, J=12.7 Hz, 0.7H), 4.70 (m, 0.7H), 4.64 (m, 0.3H), 4.45(m, 1H), 3.75 (m, 2H), 3.4-3.2 (m, 10H), 1.35 (m, 6H).

(ESI) (M+H)⁺=483.668

### N-[2-(Diethylamino)ethyl]-1-(2-furylmethyl)-4-phenyl-2,3,3a,4,5,9b-hexahydro-1H-pyrrolo[3,2-c]quinoline-8-carboxamide

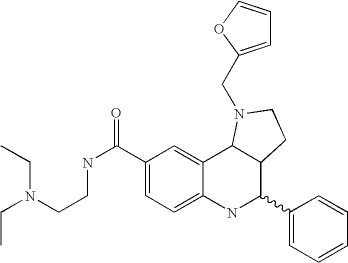 The titled compound (140 mg as TFA salt, yield: 79%) was prepared according the general procedure 8.

¹H-NMR (400 MHz, CDCl₃): 8.04 (d, J=1.6 Hz, 1H), 7.80-7.60 (m, 2H), 7.50-7.25 (m, 5H), 6.93 (d, J=8.6 Hz, 0.22H), 6.82 (d, J=8.8 Hz, 0.78H), 6.77 (m, 1H), 6.53 (m, 1H), 5.14 (d, J=9.4 HZ, 0.22H), 4.65-4.55 (m, 2H), 4.07 (d, J=11.6H, 0.78H), 3.73 (m, 2H), 3.57 (m, 2H), 3.33 (m, 10H), 3.14 (m, 0.78H), 2.20 (m, 1H), 1.32 (m, 6H). ppm. MS (ESI) (M+H)⁺=473.629.

### N-[2-(Diethylamino)ethyl]-4-phenyl-1-(pyridin-3-ylmethyl)-2,3,3a,4,5,9b-hexahydro-1H -pyrrolo[3,2-c]quinoline-8-carboxamide

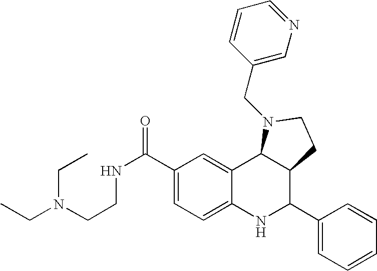 The titled compound (95.6 mg; yield: 53%) was prepared by following the general procedure 8.

¹H-NMR (400 MHz, CD3Cl): 8.65 (m, 2H), 8.10 (br, 1H), 7.92 (d, J=2.1 Hz, 0.6H), 7.78 (d, J=2.0 Hz, 0.4H), 7.64 (m, 1H), 7.56 (br, 1H), 7.34 (m, 5H), 6.86 (d, J=8.6 Hz, 0.4H), 6.74 (d, J=8.6 Hz, 0.6H), 5.13 (d, J=9.8 Hz, 0.4H), 4.93 (m, 0.6H), 4.65-4.40 (m, 2H), 4.04 (d, J=11.5 Hz, 0.4H), 3.64 (m, 2H), 3.40-3.05 (m, 10), 2.66 (m, 0.4H), 2.15 (m, 0.6H), 1.24 (m, 6H). ppm. MS (ESI) (M+H)⁺=484.648.

### N-[2-(Diethylamino)ethyl]-1-[(1-methyl-1H-pyrrolo-2-yl)methyl]-4-phenyl-2,3,3a,4,5,9b -hexahydro-1H-pyrrolo[3,2-c]quinoline-8-carboxamide

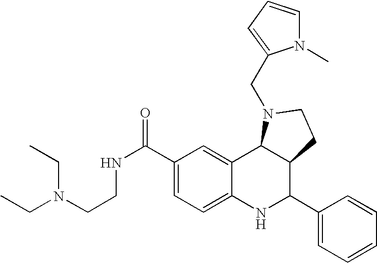 The titled compound (72 mg; yield: 40%) was prepared by following the general procedure 8.

### 1-(3-Furylmethyl)-8-(morpholin-4-ylcarbonyl)-4-phenyl-2,3,3a,4,5,9b-hexahydro-1H-pyrrolo [3,2-c]quinoline

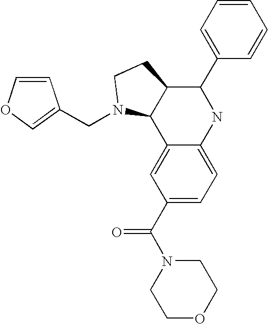 The titled compound (83.6 mg; 75% yield) was prepared according the general procedure 8.

¹HNMR (400 MHz, CDCl₃): 7.50-7.30 (m, 8H), 7.25-7.10 (1.38H), 6.55 (d, J=8.2 HZ, 1H), 6.35 (m, 0.75H), 4.26 (d, J=−12 HZ, 1H), 4.08 (d, J=−12 Hz, 1H), 3.40-3.85 (m, 8H), 3.28 (d, J=5.1 Hz, 0.75H), 3.20 (m, 1.50H), 3.08 (dt, J=9.3, 4.1 Hz, 0.75H), 2.40-2.20 (m, 2H), 1.85-1.70 (m, 1H), 1.60-1.40 (m, 1H), ppm. MS (ESI) (M+H)⁺=443.544.

### N-[2-(Diisopropylamino)ethyl]-1-[(5-ethyl-2-furyl)methyl]-4-phenyl-2,3,3a,4,5,9b-hexahydro-1H -pyrrolo[3,2-c]quinoline-8-carboxamide

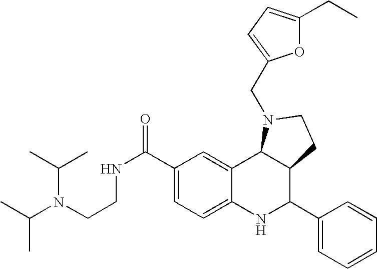 The titled compound (45 mg as TFA salt; yield: 30%) was prepared according to the general procedure 8.

MS (ESI) (M+H)⁺=529.737.

### 4-Phenyl-8-(pyrrolidin-1-ylcarbonyl)-1-(thien-2-ylmethyl)-2,3,3a,4,5,9b-hexahydro-1H-pyrrolo[3,2-c]quinoline

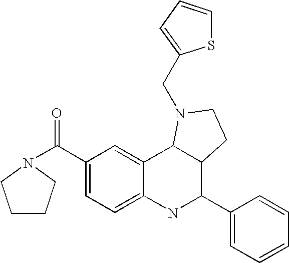 The titled compound (45.0 mg; yield: 54%) was prepared according to the general procedure 8.

¹H NMR (CD3C₁): 7.77 (s, 1H), 7.50-7.30 (m, 9H), 7.17 (dd, J=4.4, 3.4 Hz, 1H), 6.61 (d, J=8.4 Hz, 1H), 4.94 (d, J=4.2 Hz, 1H), 4.53 (d, J=4.2 Hz, 1H), 4.38 (dd, J=10.3, 6.2 Hz, 1H), 3.78(m, 1H), 3.60 (m, 4H), 3.26(m, 1H), 2.58 (m, 1H), 2.10-1.70 (m, 6H), ppm. MS (ESI) (M+H)⁺=444.612.

### N,N-Diethyl-4-phenyl-1-(thien-2-ylsulfonyl)-2,3,3a,4,5,9b-hexahydro-1H-pyrrolo[3,2-c]quinoline-8-carboxamide

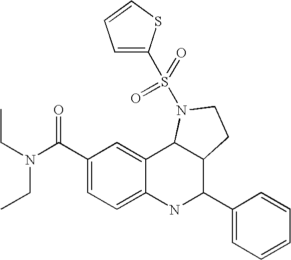 The titled compound (85 mg, 76%) was obtained by following the general procedure 10.

¹H NMR (400 MHZ, CDCl3): ppm 7.88 (0.3H,m), 7.76 (0.7H, dd, J=2.0, 1.1 Hz), 7.64 (0.3H, m), 7.63 (0.3H, m), 7.53 (0.7H, dd, J=5.1, 1.1 Hz), 7.43 (0.7H, dd, J=3.8, 1.4 Hz), 7.27 (5H,m), 7.12 (1H,m), 7.11 (1H, dd, J=7.0, 2.1 Hz), 7.05 (1H, dd, J=4.9, 3.8 Hz), 6.50 (0.7H, d, J=8.2 Hz), 6.30 (0.3H, d, J=8.0 Hz), 5.16 (0.3H, d, J=7.0 Hz), 4.65 (0.3H, d, J=2.8 Hz), 4.58 (0.7H, d, J=6.5 Hz), 4.27 (0.7H,m), 3.48 (5H,m), 1.92 (3H,m), 1.27 (2.1H, t, J=7.0 Hz), 1.28 (3.9H, t, J=7.0 Hz).

MS (ESI) (M+H)⁺=496.659.

1. A compound of formula I, a pharmaceutically acceptable salt thereof, diastereomers, enantiomers, or mixtures thereof: 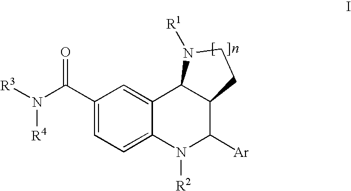
wherein
n is 1 or 2;
R¹ is selected from —H, C₁₋₆alkyl, C₂₋₆alkenyl, C₃₋₆cycloalkyl, —CH₂—R⁸, —C(═O)—NH—R⁷, —C(═S)—NH—R⁷, —C(═O)—O—R⁷—S(═O)₂—R⁶, and —C(═O)—R⁵, wherein R⁵, R⁶, R⁷ and R⁸ are independently selected from C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl used in defining R¹, R⁵, R⁶, R⁷ or R⁸ are optionally substituted with one or more groups selected from —OH, —CHO, —NH₂, —NHR, —NR₂, C₁₋₆alkyl, —C(═O)—R, —C(═O)—OR, —C(═O)—NHR, —SR, —SH, halogenated C₁₋₆alkyl, —CN, —NO₂, C₁₋₆alkoxy and halogen, or disubstituted with —O—CH₂—O— to form a fused ring;
R² is selected from —H and C₁₋₆alkyl;
R³ and R⁴ are independently selected from —H, C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy and halogen; or R³ and R⁴ together with the nitrogen connected thereto in formula I form a heterocycle ring, wherein said heterocycle ring is optionally substituted with one or more groups selected from benzyl, —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen;
Ar is selected from C₆₋₁₀aryl and C₃₋₆heteroaryl, wherein said C₆₋₁₀aryl and C₃₋₆heteroaryl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen; and
R is C₁₋₆alkyl.

2. A compound according to claim 1,
wherein n is 1 or 2;
R¹ is selected from C₁₋₆alkyl, C₂₋₆alkenyl, C₃₋₆cycloalkyl, —CH₂—R⁸, —C(═O)—NH—R⁷, —C(═S)—NH—R⁷, —S(═O)₂—R⁶, and —C(═O)—R⁵, wherein R⁵, R⁶, R⁷ and R⁸ are independently selected from C₁₋₆alkyl, C₂₋₆alkenyl, C₃₋₆cycloalkyl, C₃₋₆cycloalkyl-C₁₋₂alkyl, phenyl, phenyl-C₁₋₂alkyl, C₃₋₆heterocycloalkyl, C₃₋₆heterocycloalkyl-C₁₋₂alkyl, C₃₋₆heteroaryl, and C₃₋₆heteroaryl-C₁₋₂alkyl, wherein said C₁₋₄alkyl, C₂₋₄alkenyl, C₃₋₆alkyl, phenyl, phenyl-C₁₋₂alkyl, C₃₋₆heterocycloalkyl, C₃₋₆heterocycloalkyl-C₁₋₂alkyl, C₃₋₆heteroaryl, and C₃₋₆heteroaryl-C₁₋₂alkyl used in defining R¹, R⁵, R⁶, R⁷ or R⁸ are optionally substituted with one or more groups selected from —OH, —CHO, —NH₂, —NHR, —NR₂, C₁₋₃alkyl, —C(═O)—R, —C(═O)—OR, —SR, —CF₃, —CN, methoxy, ethoxy, fluoro and chloro, or disubstituted with —O—CH₂—O— to form a fused ring;
R² is selected from —H, methyl and ethyl;
R³ and R⁴ are independently selected from —H, C₁₋₄alkyl, C₂₋₄alkenyl, C₃₋₆cycloalkyl, C₃₋₆cycloalkyl-C₁₋₂alkyl, phenyl, phenyl-C₁₋₂alkyl, C₃₋₆heterocycloalkyl, C₃₋₆heterocycloalkyl-C₁₋₂alkyl, C₃₋₆heteroaryl, and C₃₋₆heteroaryl-C₁₋₂alkyl, wherein said C₁₋₄alkyl, C₂₋₄alkenyl, C₃₋₆cycloalkyl, C₃₋₆cycloalkyl-C₁₋₂alkyl, phenyl, phenyl-C₁₋₂alkyl, C₃₋₆heterocycloalkyl, C₃₋₆heterocycloalkyl-C₁₋₂alkyl, C₃₋₆heteroaryl, and C₃₋₆heteroaryl-C₁₋₂alkyl are optionally substituted with one or more groups selected from —CHO, —NH₂, —NHR, —NR₂, C₁₋₃alkyl, —C(═O)—OR, —CF₃, —CN, methoxy, ethoxy, fluoro and chloro; or R³ and R⁴ together with the nitrogen connected thereto in formula I form a heterocycloalkyl ring, wherein said heterocycloalkyl ring is optionally substituted with one or more groups selected from benzyl, —CHO, C₁₋₃alkyl, —C(═O)—OR, —CF₃, —CN, methoxy, ethoxy, fluoro and chloro;
Ar is selected from phenyl and five or six-membered C₃₋₅heteroaryl, wherein said phenyl and five or six-membered C₃₋₅heteroaryl are optionally substituted with one or more groups selected from C₁₋₃alkyl, —C(═O)—OR, —CF₃, —CN, methoxy, ethoxy, fluoro and chloro; and
R is C₁₋₃alkyl.

3. A compound according to claim 1,
wherein n is 1 or 2;
R¹ is selected from —CH₂—R⁸, —C(═O)—NH—R⁷, —C(═S)—NH—R⁷—S(═O)₂—R⁶ and —C(═O)—R⁵, wherein R⁵, R⁶, R⁷ and R⁸ are independently selected from C₁₋₆alkyl, C₂₋₆alkenyl, C₃₋₆cycloalkyl, C₃₋₆cycloalkyl-C₁₋₂alkyl, phenyl, benzyl, C₃₋₆heterocycloalkyl, C₃₋₆heterocycloalkyl-C₁₋₂alkyl, C₃₋₆heteroaryl, wherein said C₁₋₆alkyl, C₂₋₆alkenyl, C₃₋₆cycloalkyl, C₃₋₆cycloalkyl-C₁₋₂alkyl, phenyl, benzyl, C₃₋₆heterocycloalkyl, C₃₋₆heterocycloalkyl-C₁₋₂alkyl, C₃₋₆heteroaryl are optionally substituted with one or more groups selected from methyl, ethyl, —C(═O)—CH₃, —C(═O)—OCH₃, —C(═O)—OCH₂—CH₃, —SCH₃, —CN, methoxy, ethoxy, fluoro and chloro, or said phenyl or benzyl is optionally disubstituted with —O—CH₂—O— to form a fused ring;
R² is selected from —H, methyl and ethyl;
R³ and R⁴ are independently selected from —H, methyl, ethyl, propenyl, cyclopropyl-methyl, cyclobutyl, cyclopentyl, tetrahydrofuryl-methyl, furyl-methyl, pyridyl-methyl, thiomorpholinyl-ethyl, pyrrolidinyl-methyl, pyrrolidinyl-ethyl, thienyl-methyl, wherein said methyl, ethyl, propenyl, cyclopropyl-methyl, cyclobutyl, cyclopentyl, tetrahydrofuryl-methyl, furyl-methyl, pyridyl-methyl, thiomorpholinyl-ethyl, pyrrolidinyl-methyl, pyrrolidinyl-ethyl, thienyl-methyl are optionally substituted with one or more groups selected from dimethylamino, diethylamino, diisopropylamino, methyl, ethyl, methoxy, or R³ and R⁴ together with the nitrogen connected thereto in formula I form a heterocycloalkyl ring selected from piperidine, azetidine, piperazine, pyrrolidine and morpholine, wherein said piperidine, azetidine, piperazine, pyrrolidine and morpholine is optionally substituted with one or more groups selected from benzyl, methyl and —CHO; and
Ar is selected from phenyl, pyridyl, furyl and thienyl, wherein said phenyl, pyridyl, furyl and thienyl are optionally substituted with one or more methoxy or ethoxy.

4. A compound according to claim 1,
wherein n is 1 or 2;
R¹ is selected from —CH₂—R⁸, —C(═O)—NH—R⁷, —C(═S)—NH—R⁷—S(═O)₂—R⁶ and —C(═O)—R⁵, wherein R⁵, R⁶, R⁷ and R⁸ are independently selected from methyl, ethyl, isopropyl, 1-propyl, 2-methyl-1-propyl, 3-methyl-1-butyl, 2-ethyl-1-butyl, 1-butyl, 1-propen-3-yl, 4-methyl-2-penten-1-yl, 3-methyl-2-buten-1-yl, cyclopropyl, cyclobutyl, cyclopentyl, cyclohexyl, cyclopentyl-methyl, phenyl, benzyl, 4-morpholinyl-ethyl, tetrahydrothiopyran-4-yl-ethyl, furyl, isoxazolyl, pyridyl, thienyl, pyrazolyl, imidazolyl, and pyrrolyl, wherein said methyl, ethyl, isopropyl, 1-propyl, 2-methyl-1-propyl, 3-methyl-1-butyl, 2-ethyl-1-butyl, 1-butyl, 1-propen-3-yl, 4-methyl-2-penten-1-yl, 3-methyl-2-buten-1-yl, cyclopropyl, cyclobutyl, cyclopentyl, cyclohexyl, cyclopentyl-methyl, phenyl, benzyl, 4-morpholinyl-ethyl, tetrahydrothiopyran-4-yl-ethyl, furyl, isoxazolyl, pyridyl, thienyl, pyrazolyl, imidazolyl, and pyrrolyl are optionally substituted with one or more groups selected from methyl, ethyl, —C(═O)—CH₃, —C(═O)—OCH₃, —C(═O)—OCH₂—CH₃, —SCH₃, —CN, methoxy, ethoxy, fluoro and chloro, or said phenyl or benzyl is optionally disubstituted with —O—CH₂—O— to form a fused ring;
R² is selected from —H, methyl and ethyl;
R³ and R⁴ are independently selected from —H, methyl, ethyl, propenyl, cyclopropyl-methyl, cyclobutyl, cyclopentyl, tetrahydrofuryl-methyl, furyl-methyl, pyridyl-methyl, thiomorpholinyl-ethyl, pyrrolidinyl-methyl, pyrrolidinyl-ethyl, thienyl-methyl, wherein said methyl, ethyl, propenyl, cyclopropyl-methyl, cyclobutyl, cyclopentyl, tetrahydrofuryl-methyl, furyl-methyl, pyridyl-methyl, thiomorpholinyl-ethyl, pyrrolidinyl-methyl, pyrrolidinyl-ethyl, thienyl-methyl are optionally substituted with one or more groups selected from dimethylamino, diethylamino, diisopropylamino, methyl, ethyl, methoxy, or R³ and R⁴ together with the nitrogen connected thereto in formula I form a heterocycloalkyl ring selected from piperidine, azetidine, piperazine, pyrrolidine and morpholine, wherein said piperidine, azetidine, piperazine, pyrrolidine and morpholine is optionally substituted with one or more groups selected from benzyl, methyl and —CHO; and
Ar is selected from phenyl, 4-ethoxyphenyl, 4-methoxyphenyl, pyridyl, furyl and thienyl.

5. A compound according to claim 1, wherein the compound is selected from:
1-Benzoyl-4-phenyl-8-(pyrrolidin-1-ylcarbonyl)-2,3,3a,4,5,9b-hexahydro-1H-pyrrolo[3,2-c]quinoline;
1-Benzoyl-N-[2-(diethylamino)ethyl]-4-phenyl-2,3,3a,4,5,9b-hexahydro-1H-pyrrolo[3,2-c]quinoline-8-carboxamide;
N,N-Diethyl-4-phenyl-1-(phenylsulfonyl)-2,3,3a,4,5,9b-hexahydro-1H-pyrrolo[3,2-c]quinoline-8-carboxamide;
1-Benzyl-N-[2-(diethylamino)ethyl]-4-phenyl-2,3,3a,4,5,9b-hexahydro-1H-pyrrolo[3,2-c]quinoline-8-carboxamide;
N-[2-(Diethylamino)ethyl]-1-(2-furylmethyl)-4-phenyl-2,3,3a,4,5,9b-hexahydro-1H-pyrrolo[3,2-c]quinoline-8-carboxamide;
N-[2-(Diethylamino)ethyl]-4-phenyl-1-(pyridin-3-ylmethyl)-2,3,3a,4,5,9b-hexahydro-1H-pyrrolo[3,2-c]quinoline-8-carboxamide;
N-[2-(Diethylamino)ethyl]-1-[(1-methyl-1H-pyrrolo-2-yl)methyl]-4-phenyl-2,3,3a,4,5,9b-hexahydro-1H-pyrrolo[3,2-c]quinoline-8-carboxamide;
1-(3-Furylmethyl)-8-(morpholin-4-ylcarbonyl)-4-phenyl-2,3,3a,4,5,9b-hexahydro-1H-pyrrolo[3,2-c]quinoline;
N-[2-(Diisopropylamino)ethyl]-1-[(5-ethyl-2-furyl)methyl]-4-phenyl-2,3,3a,4,5,9b-hexahydro-1H -pyrrolo[3,2-c]quinoline-8-carboxamide;
4-Phenyl-8-(pyrrolidin-1-ylcarbonyl)-1-(thien-2-ylmethyl)-2,3,3a,4,5,9b-hexahydro-1H-pyrrolo[3,2-c]quinoline;
N,N-Diethyl-4-phenyl-1-(thien-2-ylsulfonyl)-2,3,3a,4,5,9b-hexahydro-1H-pyrrolo[3,2-c]quinoline-8-carboxamide;
and pharmaceutically acceptable salts thereof.

6-7. (canceled)

8. A pharmaceutical composition comprising a compound according to claim 1 and a pharmaceutically acceptable carrier.

9. A method for the therapy of pain in a warm-blooded animal, comprising the step of administering to said animal in need of such therapy a therapeutically effective amount of a compound according to claim 1.

10. A method for the therapy of functional gastrointestinal disorders in a warm-blooded animal, comprising the step of administering to said animal in need of such therapy a therapeutically effective amount of a compound according to claim 1.

11. A process for preparing a compound of formula I, comprising: 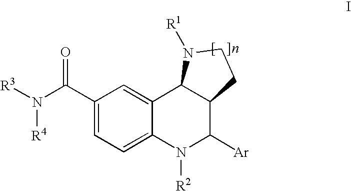
reacting a compound of formula II with a compound selected from R⁵—C(═O)—Cl, R⁶—S(═O)₂—Cl, R⁷—NCO, R⁷—NCS and R⁸CHO: 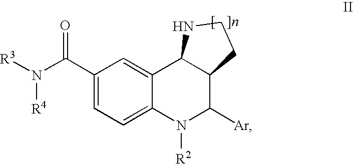
wherein
n is 1 or 2;
R¹ is selected from —CH₂—R⁸—C(═O)—NH—R⁷—C(═S)—NH—R⁷—S(═O)₂—R⁶ and —C(═O)—R⁵ wherein R⁵, R⁶, R⁷ and R⁸ are independently selected from C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl are optionally substituted with one or more groups selected from —OH, —CHO, —NH₂, —NHR, —NR₂, C₁₋₆alkyl, —C(═O)—R, —C(═O)—OR, —C(═O)—NHR, —SR, —SH, halogenated C₁₋₆alkyl, —CN, —NO₂, C₁₋₆alkoxy and halogen, or disubstituted with —O—CH₂—O— to form a fused ring;
R² is selected from —H and C₁₋₆alkyl;
R³ and R⁴ are independently selected from —H, C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy and halogen; or R³ and R⁴ together with the nitrogen connected thereto in formula I form a heterocycle ring, wherein said heterocycle ring is optionally substituted with one or more groups selected from benzyl, —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen;
Ar is selected from C₆₋₁₀aryl and C₃₋₆heteroaryl, wherein said C₆₋₁₀aryl and C₃₋₆heteroaryl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen; and
R is C₁₋₆alkyl.

12. A process for preparing a compound of formula I, comprising: 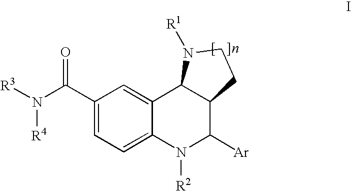
reacting a compound of formula III with R³R⁴NH: 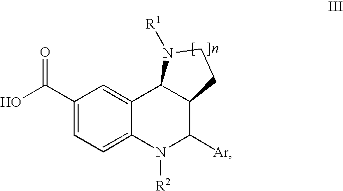
wherein
n is 1 or 2;
R¹ is selected from —C(═O)—O—C₁₋₆alkyl and —C(═O)—O—C₂₋₆alkenyl;
R² is selected from —H and C₁₋₆alkyl;
R³ and R⁴ are independently selected from —H, C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy and halogen; or R³ and R⁴ together with the nitrogen connected thereto in formula I form a heterocycle ring, wherein said heterocycle ring is optionally substituted with one or more groups selected from benzyl, —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen;
Ar is selected from C₆₋₁₀aryl and C₃₋₆heteroaryl, wherein said C₆₋₁₀aryl and C₃₋₆heteroaryl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen; and
R is C₁₋₆alkyl.

13. A process for preparing a compound of formula IV, comprising: 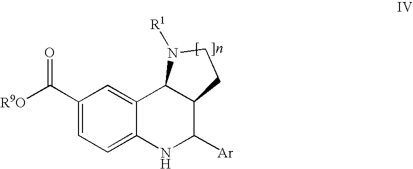
reacting a compound of formula V with a compound of formula VI: 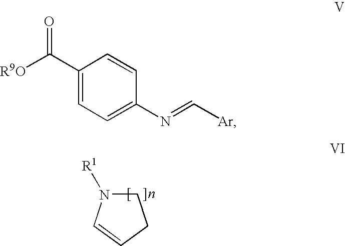
wherein
n is 1 or 2;
R¹ is selected from —C(═O)—O—C₁₋₆alkyl and —C(═O)—O—C₂₋₆alkenyl;
R⁹ is C₁₋₆alkyl;
Ar is selected from C₆₋₁₀aryl and C₃₋₆heteroaryl, wherein said C₆₋₁₀aryl and C₃₋₆heteroaryl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen; and
R is C₁₋₆alkyl.

14. A compound of formula II: 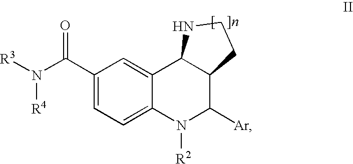
wherein
n is 1 or 2;
R² is selected from —H and C₁₋₆alkyl;
R³ and R⁴ are independently selected from —H, C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl, wherein said C₁₋₆alkyl, C₂₋₆alkenyl, C₃₋₆cycloalkyl, C₃₋₆cycloalkyl-C₁₋₄alkyl, C₆₋₁₀aryl, C₆₋₁₀aryl-C₁₋₄alkyl, C₃₋₆heterocycloalkyl, C₃₋₆heterocycloalkyl-C₁₋₄alkyl, C₃₋₆heteroaryl, and C₃₋₆heteroaryl-C₁₋₄alkyl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy and halogen; or R³ and R⁴ together with the nitrogen connected thereto in formula I form a heterocycle ring, wherein said heterocycle ring is optionally substituted with one or more groups selected from benzyl, —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen;
Ar is selected from C₆₋₁₀aryl and C₃₋₆heteroaryl, wherein said C₆₋₁₀aryl and C₃₋₆heteroaryl are optionally substituted with one or more groups selected from —OH, —CHO, —NH₂, —NHR, —NR₂, C₁₋₆alkyl, —C(═O)—OR, —C(═O)—NHR, —SR, —SH, halogenated C₁₋₆alkyl, —CN, —NO₂, C₁₋₆alkoxy, and halogen; and
R is C₁₋₆alkyl.

15. A compound according to claim 14, wherein the compound is selected from:
8-[(4-Methylpiperazin-1-yl)carbonyl]-4-phenyl-2,3,3a,4,5,9b-hexahydro-1H-pyrrolo[3,2-c]quinoline;
8-(Morpholin-4-ylcarbonyl)-4-phenyl-2,3,3a,4,5,9b-hexahydro-1H-pyrrolo[3,2-c]quinoline;
4-Phenyl-8-(pyrrolidin-1-ylcarbonyl)-2,3,3a,4,5,9b-hexahydro-1H-pyrrolo[3,2-c]quinoline;
N-(Cyclopropylmethyl)-4-phenyl-2,3,3a,4,5,9b-hexahydro-1H-pyrrolo[3,2-c]quinoline-8-carboxamide;
4-Phenyl-N-(tetrahydrofuran-2-ylmethyl)-2,3,3a,4,5,9b-hexahydro-1H-pyrrolo[3,2-c]quinoline-8-carboxamide;
N-(2-Methoxyethyl)-4-phenyl-2,3,3a,4,5,9b-hexahydro-1H-pyrrolo[3,2-c]quinoline-8-carboxamide;
N-[2-(Diethylamino)ethyl]-4-phenyl-2,3,3a,4,5,9b-hexahydro-1H-pyrrolo[3,2-c]quinoline-8-carboxamide;
N,N-Diethyl-4-phenyl-2,3,3a,4,5,9b-hexahydro-1H-pyrrolo[3,2-c]quinoline-8-carboxamide;
4-(4-Ethoxyphenyl)-8-[(4-methylpiperazin-1-yl)carbonyl]-2,3,3a,4,5,9b-hexahydro-1H-pyrrolo[3,2-c]quinoline;
4-(4-Ethoxyphenyl)-8-(morpholin-4-ylcarbonyl)-2,3,3a,4,5,9b-hexahydro-1H-pyrrolo[3,2-c]quinoline;
4-(4-Ethoxyphenyl)-8-(pyrrolidin-1-ylcarbonyl)-2,3,3a,4,5,9b-hexahydro-1H-pyrrolo[3,2-c]quinoline;
N-(Cyclopropylmethyl)-4-(4-ethoxyphenyl)-2,3,3a,4,5,9b-hexahydro-1H-pyrrolo[3,2-c]quinoline-8-carboxamide;
4-(4-Ethoxyphenyl)-N-(2-furylmethyl)-N-methyl-2,3,3a,4,5,9b-hexahydro-1H-pyrrolo[3,2-c]quinoline-8-carboxamide;
N-(2-Methoxyethyl)-4-phenyl-2,3,3a,4,5,9b-hexahydro-1H-pyrrolo[3,2-c]quinoline-8-carboxamide;
N-[2-(Diethylamino)ethyl]-4-(4-ethoxyphenyl)-2,3,3a,4,5,9b-hexahydro-1H-pyrrolo[3,2-c]quinoline-8-carboxamide;
(4-(4-Ethoxyphenyl)-N,N-diethyl-2,3,3a,4,5,9b-hexahydro-1H-pyrrolo[3,2-c]quinoline-8-carboxamide;
N-[2-(Diethylamino)ethyl]-4-phenyl-2,3,3a,4,5,9b-hexahydro-1H-pyrrolo[3,2-c]quinoline-8-carboxamide;
Piperazine, 1-[(2,3,3a,4,5,9b-hexahydro-4-phenyl-1H-pyrrolo[3,2-c]quinolin-8-yl)carbonyl]-4-methyl-;
Piperazine, 1-[[2,3,3a,4,5,9b-hexahydro-4-(4-methoxyphenyl)-1H-pyrrolo[3,2-c]quinolin-8-yl]carbonyl]-4-methyl-;
Piperazine, 1-[[2,3,3a,4,5,9b-hexahydro-4-(2-pyridinyl)-1H-pyrrolo[3,2-c]quinolin-8-yl]carbonyl]-4-methyl-;
1H-Pyrrolo[3,2-c]quinoline-8-carboxamide, N-[(1-ethyl-2-pyrrolidinyl)methyl]-2,3,3a,4,5,9b-hexahydro-4-phenyl-;
1H-Pyrrolo[3,2-c]quinoline-8-carboxamide, N-[2-(diethylamino)ethyl]-2,3,3a,4,5,9b-hexahydro-4-(4-methoxyphenyl)-;
1H-Pyrrolo[3,2-c]quinoline-8-carboxamide, N-[(1-ethyl-2-pyrrolidinyl)methyl]-2,3,3a,4,5,9b-hexahydro-4-(2-pyridinyl)-;
1H-Pyrrolo[3,2-c]quinoline-8-carboxamide, N-[(1-ethyl-2-pyrrolidinyl)methyl]-2,3,3a,4,5,9b-hexahydro-4-(4-methoxyphenyl)-;
1H-Pyrrolo[3,2-c]quinoline-8-carboxamide, 2,3,3a,4,5,9b-hexahydro-4-(4-methoxyphenyl)-N-(2-pyridinylmethyl)-;
1H-Pyrrolo[3,2-c]quinoline-8-carboxamide, 2,3,3a,4,5,9b-hexahydro-4-phenyl-N-(2-pyridinylmethyl)-;
1H-Pyrrolo[3,2-c]quinoline-8-carboxamide, 2,3,3a,4,5,9b-hexahydro-4-(2-pyridinyl)-N-(2-pyridinylmethyl)-;
1H-Pyrrolo[3,2-c]quinoline-8-carboxamide, N-[2-(diethylamino)ethyl]-2,3,3a,4,5,9b-hexahydro-4-(2-pyridinyl)-;
1-Piperazinecarboxaldehyde, 4-[(2,3,3a,4,5,9b-hexahydro-4-phenyl-1H-pyrrolo[3,2-c]quinolin-8-yl)carbonyl]-;
1-Piperazinecarboxaldehyde, 4-[[2,3,3a,4,5,9b-hexahydro-4-(2-pyridinyl)-1H-pyrrolo[3,2-c]quinolin-8-yl]carbonyl]-;
Piperazine, 1-[(2,3,3a,4,5,9b-hexahydro-4-phenyl-1H-pyrrolo[3,2-c]quinolin-8-yl)carbonyl]-4-(phenylmethyl)-;
Piperazine, 1-[[2,3,3a,4,5,9b-hexahydro-4-(2-pyridinyl)-1H-pyrrolo[3,2-c]quinolin-8-yl]carbonyl]-4-(phenylmethyl)-;
1H-Pyrrolo[3,2-c]quinoline-8-carboxamide, N-[2-[bis(1-methylethyl)amino]ethyl]-2,3,3a,4,5,9b-hexahydro-4-phenyl-;
1H-Pyrrolo[3,2-c]quinoline-8-carboxamide, N-[2-[bis(1-methylethyl)amino]ethyl]-2,3,3a,4,5,9b-hexahydro-4-(2-pyridinyl)-;
1H-Pyrrolo[3,2-c]quinoline-8-carboxamide, N-[2-(dimethylamino)ethyl]-2,3,3a,4,5,9b-hexahydro-4-(2-pyridinyl)-;
1H-Pyrrolo[3,2-c]quinoline-8-carboxamide, N-[2-(dimethylamino)ethyl]-2,3,3a,4,5,9b-hexahydro-4-phenyl-;
1H-Pyrrolo[3,2-c]quinoline-8-carboxamide, N-[2-(diethylamino)ethyl]-2,3,3a,4,5,9b-hexahydro-N-methyl-4-phenyl-;
1H-Pyrrolo[3,2-c]quinoline-8-carboxamide, N-[2-(diethylamino)ethyl]-2,3,3a,4,5,9b-hexahydro-N-methyl-4-(2-pyridinyl)-;
1H-Pyrrolo[3,2-c]quinoline-8-carboxamide, 2,3,3a,4,5,9b-hexahydro-4-phenyl-N-[2-(4-thiomorpholinyl)ethyl]-;
1H-Pyrrolo[3,2-c]quinoline-8-carboxamide, 2,3,3a,4,5,9b-hexahydro-4-(2-pyridinyl)-N-[2-(4-thiomorpholinyl)ethyl]-;
Benzo[h][1,6]naphthyridine-9-carboxamide, 5-(4-ethoxyphenyl)-1,2,3,4,4a,5,6,10b-octahydro-N-(2-methoxyethyl)-;
Benzo[h][1,6]naphthyridine-9-carboxamide, N-cyclopentyl-5-(4-ethoxyphenyl)-1,2,3,4,4a,5,6,10b -octahydro-;
Benzo[h][1,6]naphthyridine-9-carboxamide, N-cyclopropyl-5-(4-ethoxyphenyl)-1,2,3,4,4a,5,6,10b -octahydro-;
Benzo[h][1,6]naphthyridine-9-carboxamide, 5-(4-ethoxyphenyl)-1,2,3,4,4a,5,6,10b-octahydro-N-(2-thienylmethyl)-;
Benzo[h][1,6]naphthyridine-9-carboxamide, 5-(4-ethoxyphenyl)-1,2,3,4,4a,5,6,10b-octahydro-N-[(5-methyl-2-furanyl)methyl]-;
Benzo[h][1,6]naphthyridine-9-carboxamide, 5-(4-ethoxyphenyl)-N,N-diethyl-1,2,3,4,4a,5,6,10b -octahydro-;
Benzo[h][1,6]naphthyridine-9-carboxamide, 5-(4-ethoxyphenyl)-1,2,3,4,4a,5,6,10b-octahydro-N-[2-(1-pyrrolidinyl)ethyl]-;
Pyrrolidine, 1-[(1,2,3,4,4a,5,6,10b-octahydro-5-phenylbenzo[h][1,6]naphthyridin-9-yl)carbonyl]-;
Benzo[h][1,6]naphthyridine-9-carboxamide, 1,2,3,4,4a,5,6,10b-octahydro-N-(2-methoxyethyl)-5-phenyl-;
Benzo[h][1,6]naphthyridine-9-carboxamide, N-cyclopentyl-1,2,3,4,4a,5,6,10b-octahydro-5-phenyl-;
Benzo[h][1,6]naphthyridine-9-carboxamide, N-cyclopropyl-1,2,3,4,4a,5,6,10b-octahydro-5-phenyl-;
Benzo[h][1,6]naphthyridine-9-carboxamide, 1,2,3,4,4a,5,6,10b-octahydro-5-phenyl-N-(2-thienylmethyl)-;
Benzo[h][1,6]naphthyridine-9-carboxamide, 1,2,3,4,4a,5,6,10b-octahydro-N-[(5-methyl-2-furanyl)methyl]-5-phenyl-;
Benzo[h][1,6]naphthyridine-9-carboxamide, N,N-diethyl-1,2,3,4,4a,5,6,10b-octahydro-5-phenyl-;
Benzo[h][1,6]naphthyridine-9-carboxamide, 1,2,3,4,4a,5,6,10b-octahydro-5-phenyl-N-[2-(1-pyrrolidinyl)ethyl]-;
Pyrrolidine, 1-[(6-ethyl-1,2,3,4,4a,5,6,10b-octahydro-5-phenylbenzo[h][1,6]naphthyridin-9-yl)carbonyl]-;
Benzo[h][1,6]naphthyridine-9-carboxamide, 6-ethyl-1,2,3,4,4a,5,6,10b-octahydro-N-(2-methoxyethyl)-5-phenyl-;
Benzo[h][1,6]naphthyridine-9-carboxamide, N-cyclopentyl-6-ethyl-1,2,3,4,4a,5,6,10b-octahydro-5-phenyl-;
N-Cyclopropyl-6-ethyl-5-phenyl-1,2,3,4,4a,5,6,10b-octahydrobenzo[h]-1,6-naphthyridine-9-carboxamide;
6-Ethyl-5-phenyl-N-(thien-2-ylmethyl)-1,2,3,4,4a,5,6,10b-octahydrobenzo[h]-1,6-naphthyridine-9-carboxamide;
6-Ethyl-N-[(5-methyl-2-furyl)methyl]-5-phenyl-1,2,3,4,4a,5,6,10b-octahydrobenzo[h]-1,6-naphthyridine-9-carboxamide;
N,N,6-Triethyl-5-phenyl-1,2,3,4,4a,5,6,10b-octahydrobenzo[h]-1,6-naphthyridine-9-carboxamide;
6-Ethyl-5-phenyl-N-(2-pyrrolidin-1-ylethyl)-1,2,3,4,4a,5,6,10b-octahydrobenzo[h]-1,6-naphthyridine-9-carboxamide;
4-(4-Ethoxyphenyl)-N,N-dimethyl-2,3,3a,4,5,9b-hexahydro-1H-pyrrolo[3,2-c]quinoline-8-carboxamide;
4-(4-Ethoxyphenyl)-N-methyl-2,3,3a,4,5,9b-hexahydro-1H-pyrrolo[3,2-c]quinoline-8-carboxamide;
N-(Cyclopropylmethyl)-4-(4-ethoxyphenyl)-2,3,3a,4,5,9b-hexahydro-1H-pyrrolo[3,2-c]quinoline-8-carboxamide;
N-Cyclobutyl-4-(4-ethoxyphenyl)-2,3,3a,4,5,9b-hexahydro-1H-pyrrolo[3,2-c]quinoline-8-carboxamide;
N-Cyclopropyl-4-(4-ethoxyphenyl)-2,3,3a,4,5,9b-hexahydro-1H-pyrrolo[3,2-c]quinoline-8-carboxamide;
N-Allyl-4-(4-ethoxyphenyl)-2,3,3a,4,5,9b-hexahydro-1H-pyrrolo[3,2-c]quinoline-8-carboxamide;
4-(4-Ethoxyphenyl)-8-(piperidin-1-ylcarbonyl)-2,3,3a,4,5,9b-hexahydro-1H-pyrrolo[3,2-c]quinoline;
8-(Azetidin-1-ylcarbonyl)-4-(4-ethoxyphenyl)-2,3,3a,4,5,9b-hexahydro-1H-pyrrolo[3,2-c]quinoline;
N,N-Dimethyl-4-phenyl-2,3,3a,4,5,9b-hexahydro-1H-pyrrolo[3,2-c]quinoline-8-carboxamide;
N-Methyl-4-phenyl-2,3,3a,4,5,9b-hexahydro-1H-pyrrolo[3,2-c]quinoline-8-carboxamide;
N-(Cyclopropylmethyl)-4-phenyl-2,3,3a,4,5,9b-hexahydro-1H-pyrrolo[3,2-c]quinoline-8-carboxamide;
N-Cyclobutyl-4-phenyl-2,3,3a,4,5,9b-hexahydro-1H-pyrrolo[3,2-c]quinoline-8-carboxamide;
N-Cyclopropyl-4-phenyl-2,3,3a,4,5,9b-hexahydro-1H-pyrrolo[3,2-c]quinoline-8-carboxamide;
(N-Allyl-4-phenyl-2,3,3a,4,5,9b-hexahydro-1H-pyrrolo[3,2-c]quinoline-8-carboxamide;
4-Phenyl-8-(piperidin-1-ylcarbonyl)-2,3,3a,4,5,9b-hexahydro-1H-pyrrolo[3,2-c]quinoline;
8-(Azetidin-1-ylcarbonyl)-4-phenyl-2,3,3a,4,5,9b-hexahydro-1H-pyrrolo[3,2-c]quinoline;
4-(2-Furyl)-N,N-dimethyl-2,3,3a,4,5,9b-hexahydro-1H-pyrrolo[3,2-c]quinoline-8-carboxamide;
4-(2-Furyl)-N-methyl-2,3,3a,4,5,9b-hexahydro-1H-pyrrolo[3,2-c]quinoline-8-carboxamide;
N-(Cyclopropylmethyl)-4-(2-furyl)-2,3,3a,4,5,9b-hexahydro-1H-pyrrolo[3,2-c]quinoline-8-carboxamide;
N-Cyclobutyl-4-(2-furyl)-2,3,3a,4,5,9b-hexahydro-1H-pyrrolo[3,2-c]quinoline-8-carboxamide;
N-Cyclopropyl-4-(2-furyl)-2,3,3a,4,5,9b-hexahydro-1H-pyrrolo[3,2-c]quinoline-8-carboxamide;
N-Allyl-4-(2-furyl)-2,3,3a,4,5,9b-hexahydro-1H-pyrrolo[3,2-c]quinoline-8-carboxamide;
4-(2-Furyl)-8-(piperidin-1-ylcarbonyl)-2,3,3a,4,5,9b-hexahydro-1H-pyrrolo[3,2-c]quinoline;
8-(Azetidin-1-ylcarbonyl)-4-(2-furyl)-2,3,3a,4,5,9b-hexahydro-1H-pyrrolo[3,2-c]quinoline;
N,N-Dimethyl-4-thien-3-yl-2,3,3a,4,5,9b-hexahydro-1H-pyrrolo[3,2-c]quinoline-8-carboxamide;
N-Methyl-4-thien-3-yl-2,3,3a,4,5,9b-hexahydro-1H-pyrrolo[3,2-c]quinoline-8-carboxamide;
N-(Cyclopropylmethyl)-4-thien-3-yl-2,3,3a,4,5,9b-hexahydro-1H-pyrrolo[3,2-c]quinoline-8-carboxamide;
N-Cyclobutyl-4-thien-3-yl-2,3,3a,4,5,9b-hexahydro-1H-pyrrolo[3,2-c]quinoline-8-carboxamide;
N-Cyclopropyl-4-thien-3-yl-2,3,3a,4,5,9b-hexahydro-1H-pyrrolo[3,2-c]quinoline-8-carboxamide;
N-Allyl-4-thien-3-yl-2,3,3a,4,5,9b-hexahydro-1H-pyrrolo[3,2-c]quinoline-8-carboxamide;
8-(Piperidin-1-ylcarbonyl)-4-thien-3-yl-2,3,3a,4,5,9b-hexahydro-1H-pyrrolo[3,2-c]quinoline;
8-(Azetidin-1-ylcarbonyl)-4-thien-3-yl-2,3,3a,4,5,9b-hexahydro-1H-pyrrolo[3,2-c]quinoline;
N-[2-(Dimethylamino)ethyl]-4-phenyl-2,3,3a,4,5,9b-hexahydro-1H-pyrrolo[3,2-c]quinoline-8-carboxamide; and pharmaceutically acceptable salts thereof.

16. A method for the therapy of anxiety in a warm-blooded animal, comprising the step of administering to said animal in need of such therapy a therapeutically effective amount of a compound according to claim 1.
